(19) United States
(12) Patent Application Publication
(10) Pub. No.: US 2017/0146386 A1
(43) Pub. Date: May 25, 2017

(54) SCALE-BASED USER-PHYSIOLOGICAL SOCIAL GROUPING SYSTEM

(71) Applicant: Physiowave, Inc., Santa Clara, CA (US)

(72) Inventors: Richard M. Wiard, Campbell, CA (US); Gregory T. Kovacs, Palo Alto, CA (US)

(21) Appl. No.: 15/257,538

(22) Filed: Sep. 6, 2016

Related U.S. Application Data
(60) Provisional application No. 62/258,253, filed on Nov. 20, 2015.
(60) Provisional application No. 62/266,440, filed on Dec. 11, 2015.
(60) Provisional application No. 62/266,523, filed on Dec. 11, 2015.

(51) Int. Cl. G01G 19/50; A61B 5/0205; G01G 23/36; A61B 5/00; G01G 21/22; G06F 19/00; A61B 5/117

(52) U.S. Cl. CPC G01G 19/50; G06F 19/322; G06F 19/3406; A61B 5/0205; A61B 5/117; A61B 5/7275; A61B 5/7282; G01G 21/22; G01G 23/36; A61B 5/0245

(57) ABSTRACT

Embodiments are directed to an apparatus that includes a weighing scale and external circuitry. The weighing scale includes a platform and processing circuitry. The platform includes force sensor circuitry and a plurality of electrodes integrated with the platform. The processing circuitry is electrically integrated with the force sensor circuitry and the plurality of electrodes and collect cardio-related physiologic data from the user and outputs at least portions of the cardio-related physiologic data as user data. The external circuitry receives user data from a plurality of weighing scales and pools user data for a plurality of users into user data sets for each user. The external circuitry further identifies a subset of the plurality of users with correlations, identifies and normalizes user data from the user data sets of the subsets of users based on prioritization data and normalization data, and provides the subsets of users of the social groups with access to a social group via respective scales of the subset of users.

### RELATED APPLICATION DATA

This application is related to the U.S. Provisional application (Ser. No. 62/266,440), entitled “Scale-based User-Physiological Social Grouping System”, filed Dec. 11, 2015, U.S. Provisional application (Ser. No. 62/258,238), entitled “Condition or Treatment Assessment Methods and Platform Apparatuses”, filed Nov. 20, 2015, and U.S. Provisional application (Ser. No. 62/266,523) entitled “Social Grouping Using a User-Specific Scale-Based Enterprise System”, filed Dec. 11, 2015, which are fully incorporated herein by reference.

### OVERVIEW

Various aspects of the present disclosure are directed toward methods, systems and apparatuses that are useful in a user-physiological social grouping system.

Various aspects of the present disclosure are directed to monitoring different physiological characteristics are monitored for many different applications. For instance, physiological monitoring instruments are often used to measure a number of patient vital signs, including blood oxygen level, body temperature, respiration rate and electrical activity for electrocardiograms (ECG) or electroencephalogram (EEG) measurements. For ECG measurements, a number of electrocardiograph leads may be connected to a patient's skin, and are used to obtain a signal from the patient.

Obtaining physiological signals (e.g., data) can often require specialty equipment and intervention with medical professionals. For many applications, such requirements may be costly or burdensome. These and other matters have presented challenges to monitoring physiological characteristics.

Aspects of the present disclosure are directed to a platform apparatus and external circuitry that provides user access to particular social groups based on user-physiological data, such as scale-obtained data. The platform apparatus, such as a body weight scale, provides the feature of collecting scale-obtained data including cardio-physiological measurements from a user while the user is standing on the platform apparatus and outputting the scale-obtained data to external circuitry. In specific aspects, the external circuitry includes a server CPU that pools user data from a plurality of scales and is used, in connection with the scale, to provide the users with access to social groups. The access to social groups, in various specific aspects, includes access to a forum, blog, and/or webpage of a social network that connects users of the social group. The social groups are identified automatically by the external circuitry and/or the scale based on scale-obtained data and a prompt is provided to the user, using a user interface of the scale or another graphical user interface, to communicate the availability of the social group. In various aspects, the external circuitry determines the social groups by identifying various users with risks for a condition using the scale-obtained data, diagnosis, similar parameter values, user goals, and/or various other correlations. The platform apparatus can be configured to recognize multiple users and identify the particular users to display the prompt to, using a scale-based biometric, such as a cardiogram characteristic. In other related aspects, the scale prioritizes the various multiple users and outputs the data to identify social groups, and for other services, based on the priority of the user. Further, the user inputs provided using the access to the social group is used as feedback by the external circuitry to further refine various conditions and/or risks that the user may have.

In certain aspects, the present disclosure is directed to apparatuses and methods including a scale and external circuitry. The scale includes a user display to display data to a user while the user is standing on the scale, a platform for a user to stand on, data-procurement circuitry, and processing circuitry. The data-procurement circuitry includes force sensor circuitry and a plurality of electrodes integrated with the platform for engaging the user with electrical signals and collecting signals indicative of the user's identity and cardio-physiological measurements while the user is standing on the platform. The processing circuitry includes a CPU and a memory circuit with user corresponding data stored in the memory circuit. The processing circuitry is electrically integrated with the force sensor circuitry and the plurality of electrodes and configured to process data obtained by the data-procurement circuitry while the user is standing on the platform and therefrom derive and output user data to external circuitry, including data indicative of the user's identity and the cardio-physiological measurements, for assessment at a remote location that is not integrated within the scale. The output circuitry displays the user's weight and outputs the data indicative of the user's identity and/or the user data generated cardio-related physiologic data from the scale for reception at a remote location.

The external circuitry includes processing circuitry and a memory circuit. The memory circuit stores reference information. For example, the external circuitry receives the user data and identifies a risk that the user has a condition using the reference information and the user data provided by the scale. Further, the external circuitry outputs generic health information correlating to the condition to the scale that is tailored based on the identified risk. The risk of a condition, as used herein, includes a probability that the user has the condition and a severity of the condition. For example, in various embodiments, the generic health information is output in response to the probability being greater than a threshold and the severity being greater than a threshold.

In specific aspects, an apparatus includes a weighing scale and external circuitry. The weighing scale includes a platform including force sensor circuitry and a plurality of electrodes integrated with the platform, and configured and arranged for engaging the user with electrical signals and collecting signals indicative of the user's identity and cardio-related physiologic data while the user is standing on the platform. The weighing scale further includes processing circuitry, including a CPU and a memory circuit with user-corresponding data stored in the memory circuit. The processing circuitry is arranged under the platform and electrically integrated with the force sensor circuitry and the plurality of electrodes and being configured to collect cardio-related physiologic data from the user while the user is standing on the platform and output at least portions of the cardio-related physiologic data as user data. The external circuitry configured receives user data from a plurality of weighing scales include the weighing scale and pools user data for a plurality of users of a plurality of scales into user data sets for each user. Further, the external circuitry identifies a subset of the plurality of users with one or more correlations between the user data sets based on the pooled used data, identifies and normalizes user data from the user data sets of the subset of users based on prioritization data and normalization data, and provides the subsets of users of the social groups with access to a social group via respective scales of the subset of users. The access can include selective access to the normalized user data from the user data sets. The normalized user data includes portions of user data from the user data sets selected using the priority data that is normalized using the normalization data.

In other-related and specific aspects, an apparatus includes a weighing scale and external circuitry. The weighing scale includes a platform, a user display, and processing circuitry. The platform including force sensor circuitry and a plurality of electrodes integrated with the platform, and for engaging the user with electrical signals and collecting signals indicative of the user's identity and cardio-related physiologic data while the user is standing on the platform. The user display provides data to a user while the user is standing on the scale. The processing circuitry is arranged under the platform and electrically integrated with the force sensor circuitry and the plurality of electrodes and being configured to collect cardio-related physiologic data from the user while the user is standing on the platform and output at least portions of the cardio-related physiologic data as user data. The processing circuitry further identifies the user by verifying a scale-based biometric of the user using the signals indicative of the user's identity and a user profile corresponding to the user. The external circuitry includes processing circuitry and a memory circuitry. The external circuitry receives user data from a plurality of scales, the plurality of weighing scales including the weighing scale, and pools user data for a plurality of users of a plurality of scales into user data sets for each user. Further, the external circuitry identifies a subset of the plurality of users with one or more correlations between user data sets based on the pooled used data, wherein at least one correlations includes users that are experiencing the same and/or similar symptoms, conditions, or treatments based on the user data, identifies and normalizes user data from the user data sets of the subsets of users based on prioritization data and normalization data, and provides the subsets of users of the social groups with access to a social group via respective scales of the subset of users.

The prioritization and normalization data, in various aspects, can be default values or based on user input. Prioritization data includes or refers to a prioritization of different categories of user data, including but not limited to scale-obtained physiological data, demographic data, lifestyle data (e.g., user habits include eating, drinking, smoking, sleeping, exercise, prescription medication, etc.), and diagnosis data. The categories of data can include data of different sensitivity and/or specificity levels. For example, the prioritization data can include numerical values (e.g., 1-10), binary indicators (e.g., include in social groups or not, or priority or not), and/or other ways to differentiate or group the different categories of the user data. In some specific aspects, the prioritization data can be specific to the correlation identified, and thus, a specific category of user data may have different priorities for different uses. Particular user data may be relevant (e.g., be a risk, a symptom, a way to reduce a risk or symptom, a way to improve a goal or impact a goal) to a particular correlation. As a specific example, exercise habits and age can be relevant to arterial stiffness or declining arterial compliance as they can impact the risk for the health condition and/or improvements in the risk (e.g., lower risk). The user may adjust all or portions of the prioritization data. For example, the user may select different sensitivity or specificity values, which can impact the prioritization data and/or set the prioritization data.

The normalization data can also be default or based on user input. The normalization data includes or refers to a numerical value or other privacy value to different categories of data. For example, the normalization data can include numerical values to normalize particular data to and/or normalize a privacy of different categories of data. In specific examples, user data that is provided to sets of users in a social group is normalized for privacy purposes and for sensitivity for the user. Specific users may not want their identity shown and/or may be sensitive to displaying values (such as weight or diagnosis) to different users. To protect the user's privacy and ease their comfort in using social groups, the data that users are provided access to is normalized. The normalization data can include default values and/or can be adjusted based on user input. The user can view how their data is displayed in the social group prior to providing other users with access and can further adjust the normalization. Particular data can adjusted to a numerical scale (e.g., 1-10) and/or not all of the data is displayed (e.g., don't show that the user is diagnosed with a condition). In other aspects, the data can be normalized in other ways and/or in combination. For example, instead of displaying a user's weight, the social group is provided access to a scaled version (e.g., 1-10 or 1-100) and a percentage change in the user's weight over a period of time. As another example, instead of displaying the specific diagnosis of the users (e.g., AFIB), the user is indicated as having an arrhythmia condition. The user can adjust the data displayed to the social group overtime, such as when the user becomes more comfortable with the social group. When the particular user accesses the data in the social group, other user's data is normalized based on the particular user's selection and/or based on the other users' selections.

In certain embodiments, aspects as described herein are implemented in accordance with and/or in combination with aspects of the underlying Provisional application (Ser. No. 62/266,440), entitled “Scale-based User-Physiological Social Grouping System”, filed Dec. 11, 2015, Provisional application (Ser. No. 62/258,238), entitled “Condition or Treatment Assessment Methods and Platform Apparatuses”, filed Nov. 20, 2015, and Provisional application (Ser. No. 62/266,523) entitled “Social Grouping Using a User-Specific Scale-Based Enterprise System”, filed Dec. 11, 2015 to which benefit is claimed and which are fully incorporated herein by reference.

The above discussion/summary is not intended to describe each embodiment or every implementation of the present disclosure. The figures and detailed description that follow also exemplify various embodiments.

### BRIEF DESCRIPTION OF THE DRAWINGS

Various example embodiments may be more completely understood in consideration of the following detailed description in connection with the accompanying drawings, in which:

FIG. 1a shows a scale-based user-physiological social grouping system consistent with aspects of the present disclosure;

FIG. 1b shows an example of a scale-based user-physiological heuristic system comprised of a plurality of scales and external circuitry consistent with aspects of the present disclosure;

FIG. 1c illustrates an example of providing access to social groups using a scale-based user-physiologic social grouping system consistent with aspects of the present disclosure;

FIG. 1d shows current paths through the body for the IPG trigger pulse and Foot IPG, consistent with various aspects of the present disclosure;

FIG. 1e is a flow chart illustrating an example manner in which a user-specific physiologic meter/scale may be programmed to provide features consistent with aspects of the present disclosure;

FIG. 2 shows an example of the insensitivity to foot placement on scale electrodes with multiple excitation and sensing current paths, consistent with various aspects of the present disclosure;

FIGS. 3a-3b show example block diagrams depicting circuitry for sensing and measuring the cardiovascular time-varying IPG raw signals and steps to obtain a filtered IPG waveform, consistent with various aspects of the present disclosure;

FIG. 3c depicts an example block diagram of circuitry for operating core circuits and modules, including for example those of FIGS. 3a-3b, used in various specific embodiments of the present disclosure;

FIG. 3d shows an exemplary block diagram depicting the circuitry for interpreting signals received from electrodes.

FIG. 4 shows an example block diagram depicting signal processing steps to obtain fiducial references from the individual Leg IPG “beats,” which are subsequently used to obtain fiducials in the Foot IPG, consistent with various aspects of the present disclosure;

FIG. 5 shows an example flowchart depicting signal processing to segment individual Foot IPG “beats” to produce an averaged IPG waveform of improved SNR, which is subsequently used to determine the fiducial of the averaged Foot IPG, consistent with various aspects of the present disclosure;

FIG. 6a shows examples of the Leg IPG signal with fiducials; the segmented Leg IPG into beats; and the ensemble-averaged Leg IPG beat with fiducials and calculated SNR, for an exemplary high-quality recording, consistent with various aspects of the present disclosure;

FIG. 6b shows examples of the Foot IPG signal with fiducials derived from the Leg IPG fiducials; the segmented Foot IPG into beats; and the ensemble-averaged Foot IPG beat with fiducials and calculated SNR, for an exemplary high-quality recording, consistent with various aspects of the present disclosure;

FIG. 7a shows examples of the Leg IPG signal with fiducials; the segmented Leg IPG into beats; and the ensemble averaged Leg IPG beat with fiducials and calculated SNR, for an exemplary low-quality recording, consistent with various aspects of the present disclosure;

FIG. 7b shows examples of the Foot IPG signal with fiducials derived from the Leg IPG fiducials; the segmented Foot IPG into beats; and the ensemble-averaged Foot IPG beat with fiducials and calculated SNR, for an exemplary low-quality recording, consistent with various aspects of the present disclosure;

FIG. 8 shows an example correlation plot for the reliability in obtaining the low SNR Foot IPG pulse for a 30-second recording, using the first impedance signal as the trigger pulse, from a study including 61 test subjects with various heart rates, consistent with various aspects of the present disclosure;

FIGS. 9a-b show an example configuration to obtain the pulse transit time (PTT), using the first IPG as the triggering pulse for the Foot IPG and ballistocardiogram (BCG), consistent with various aspects of the present disclosure;

FIG. 10 shows nomenclature and relationships of various cardiovascular timings, consistent with various aspects of the present disclosure;

FIG. 11 shows an example graph of PTT correlations for two detection methods (white dots) Foot IPG only, and (black dots) Dual-IPG method, consistent with various aspects of the present disclosure;

FIG. 12 shows an example graph of pulse wave velocity (PWV) obtained from the present disclosure compared to the ages of 61 human test subjects, consistent with various aspects of the present disclosure;

FIG. 13 shows another example of a scale with interleaved foot electrodes to inject and sense current from one foot to another foot, and within one foot, consistent with various aspects of the present disclosure;

FIG. 14a shows another example of a scale with interleaved foot electrodes to inject and sense current from one foot to another foot, and measure Foot IPG signals in both feet, consistent with various aspects of the present disclosure;

FIG. 14b shows another example of a scale with interleaved foot electrodes to inject and sense current from one foot to another foot, and measure Foot IPG signals in both feet, consistent with various aspects of the present disclosure;

FIG. 14c shows another example approach to floating current sources is the use of transformer-coupled current sources, consistent with various aspects of the present disclosure;

FIGS. 15a-d show an example breakdown of a scale with interleaved foot electrodes to inject and sense current from one foot to another foot, and within one foot, consistent with various aspects of the present disclosure;

FIG. 16 shows an example block diagram of circuit-based building blocks, consistent with various aspects of the present disclosure;

FIG. 17 shows an example flow diagram, consistent with various aspects of the present disclosure;

FIG. 18 shows an example scale communicatively coupled to a wireless device, consistent with various aspects of the present disclosure; and

FIGS. 19a-c show example impedance as measured through different parts of the foot based on the foot position, consistent with various aspects of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DESCRIPTION

Aspects of the present disclosure are believed to be applicable to a variety of different types of apparatuses, systems, and methods of providing user access to particular social groups based on user-physiological data, such as a forum, blog, and/or page of a social network. In certain implementations, aspects of the present disclosure have been shown to be beneficial when used in the context of prioritizing particular users of a scale for identification of correlations with others. In some embodiments, the scale outputs cardio-related physiologic data to external circuitry and the external circuitry identifies a risk that the user has a condition based on the cardio-related physiologic data and reference health information. In specific embodiments, the external circuitry identifies correlations between the data sets of different users and provides the users with correlated data sets with access to a social group. The access to the social group includes selective access to normalized user data from the user data sets. For example, the external circuitry identifies and normalizes user data from the user data sets of a sub-set of users with correlated data sets based on prioritization data and normalization data. These and other aspects can be implemented to address challenges, including those discussed in the background above. While not necessarily so limited, various aspects may be appreciated through a discussion of examples using such exemplary contexts.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

Embodiments of the present disclosure are directed to a platform apparatus and external circuitry that provide various features including grouping users based on user-physiological heuristics applied to scale-obtained data and providing the grouped users with anonymous access to social groups, such as using a forum, blog and/or social network and/or social media. The platform apparatus, such as a body weight scale, provides the features of collecting scale-obtained data including cardio-physiological measurements from a user while the user is standing on the platform apparatus and outputting the scale-obtained data to external circuitry. In various specific embodiments, the scale is configured to collect data for a plurality of users and identifies each respective user using scale-based biometrics, such as cardiogram characteristics. In specific embodiments, the external circuitry includes a server CPU that pools user data from a plurality of scales and is used, in connection with the scale, to provide the users with access to social groups. The access to social groups includes access to a forum, blog, and/or webpage of a social network (e.g., a social network or social media page) that connects users of the social group. The social groups are identified automatically by the external circuitry based on scale-obtained data. The external circuitry can determine the social groups by identifying various users with similar risk that user for a condition using the scale-obtained data, diagnosis, similar parameter values, user goals, and/or various other correlations. In various embodiments, the platform apparatus is configured to recognize multiple users and identifies the particular users to display the prompt to, using a scale-based biometric, such as a cardiogram characteristic. In other related embodiments, the scale prioritizes the various multiples users and outputs the data to identify social groups, and for other services, based on the priority of the user. The user inputs provided using the access to the social group can be used as feedback by the external circuitry to further refine various conditions and/or risks that the user may have.

Social networks, blogs, and forums are useful for users to connect with friends, family, and co-workers, and/or unknown people with similar interests or concerns. A user can use a social network to communicate personal information to other people (e.g., other users) on the social network and/or in the user's social network. For instance, the information is communicated to multiple people by the user doing a single action. The action can include a post, message, notification, and/or other action on the social network. In various instances, users group together to form discussions on various topics of interest.

Health related issues for different people can follow similar patterns. For example, users with a particular condition may have similar symptoms. Particular symptoms may occur prior to the user being diagnosed or even recognizing the symptoms. In other instances, users with similar exercise or weight goals may follow similar exercise and/or eating plans. Users having similar experiences may benefit from being grouped together in a social group, such as on a social network, to discuss symptoms, successes, failures, among other information. As the user may not recognize symptoms, they may wait longer to see a physician and/or identify that they are having a problem. Earlier detection of health related issues is beneficial for recovery, treatment/control of symptoms, and prevention of further problems. In accordance with various embodiments, scale-obtained data is pooled by external circuitry to identify various correlations between different user data sets. In response to the identified correlations, identified users with the correlation are provided with access to a social group, such as a forum, blog, and/or a page of a social network. The access to the social group can include selective access to normalized user data from the different user data sets. For example, the external circuitry identifies and normalizes portions of the user data from the user data sets of the subsets of users with an identified correlation based on prioritization data and normalization data. The user's identities remain anonymous as the data includes user-sensitive data and/or the discussions occurring on the page are sensitive to the user and the data is normalized. In this way, the user's identity is preserved while the user is participating in the social network.

In specific embodiments, the forum, blog and/or social network page automatically populates scale-obtained data from the subset of users. For instance, the forum, blog and/or social network page includes various reports and/or dashboards indicating user's successes and failures, treatments, and/or progress. The users are able to communicate about what has been helping them or not helping them to do better with symptoms of a condition, treatment, diagnosis, and/or their health goals.

The populated data is selected and includes the normalized user data. The normalized user data includes portions of user data from the user data sets selected using the priority data and that is normalized using the normalization data. For example, data from the user data sets is selectively identified and normalized based on prioritization data and normalization data. The prioritization data and normalization data can be default values or based on user input. Prioritization data includes or refers to a prioritization of different categories of user data, including but not limited to scale-obtained physiological data, demographic data, lifestyle data (e.g., user habits include eating, drinking, smoking, sleeping, exercise, prescription medication, etc.), and diagnosis data. The prioritization data can include numerical values (e.g., 1-10), binary indicators (e.g., include in social groups or not, or priority or not), and/or other ways to differentiate or group the different categories of the user data. In some specific embodiments, the prioritization data can be specific to the correlation identified, and thus, a specific category of user data may have different priorities for different uses. Particular user data may be relevant (e.g., be a risk, a symptom, a way to reduce a risk or symptom, a way to improve a goal or impact a goal) to a particular correlation. As a specific example, exercise habits and age can be relevant to arterial stiffness or declining arterial compliance as they can impact the risk for the health condition and/or improvements in the risk (e.g., lower risk). The user may adjust all or portions of the prioritization data. For example, the user may select different sensitivity or specificity values, which can impact the prioritization data and/or set the prioritization data.

The normalization data can also be default or based on user input. The normalization data includes or refers to a numerical value or other privacy value to different categories of data. For example, the normalization data can include numerical values to normalize particular user data to and/or normalize the user data for privacy of different categories of data. In specific examples, user data that is provided to subsets of users in a social group is normalized for privacy purposes and for sensitivity for the user. Specific users may not want their identity shown and/or may be sensitive to displaying values (such as weight or diagnosis) to different users. To protect the user's privacy and ease their comfort in using social groups, the user data that users are provided access to is normalized. The normalization data can include default values and/or can be adjusted based on user input. For example, the user can view how their data is displayed in the social group prior to providing other users with access and can further adjust the normalization. Particular user data can be adjusted to a numerical scale (e.g., 1-10) and/or not all of the data is displayed (e.g., don't show that the user is diagnosed with a condition). In other aspects, the user data can be normalized in other ways and/or in combination with a numerical normalization. For example, instead of displaying a user's weight, the social group is provided access to a scaled version (e.g., 1-10 or 1-100) and a percentage change in the user's weight over a period of time. As another example, instead of displaying the specific diagnosis of the users (e.g., AFIB), the user is indicated as having a general arrhythmia condition. The user can adjust the user data displayed to the social group overtime, such as when the user becomes more comfortable with the social group. When the particular user accesses the data in the social group, other user's data is normalized based on the particular user's selection and/or based on the other users' selections.

In accordance with a number of embodiments, physiological parameter data is collected using an apparatus, such as a weighing scale or other platform that the user stands on. The user or similarly-venued personnel (e.g., co-workers, friends, roommates, colleagues), may use the apparatus in the home, office, doctors office, or other such venue on a regular and frequent basis. The present disclosure is directed to a substantially-enclosed apparatus, as would be a weighing scale, wherein the apparatus includes a platform which is part of a housing or enclosure and a user-display to output user-specific information for the user while the user is standing on the platform. The apparatus is configured to collect data for a plurality of users and identify each respective user using the collected data. The platform includes a surface area with electrodes that are integrated and configured and arranged for engaging a user as he or she steps onto the platform. Within the housing is processing circuitry that includes a CPU (e.g., one or more computer processor circuits) and a memory circuit with user-corresponding data stored in the memory circuit. The platform, over which the electrodes are integrated, is integrated and communicatively connected with the processing circuitry. The processing circuitry is programmed with modules as a set of integrated circuitry which is configured and arranged for automatically obtaining a plurality of measurement signals (e.g., signals indicative of cardio-physiological measurements) from the plurality of electrodes. The processing circuitry generates, from the signals, cardio-related physiologic data manifested as user data.

The scale can include output circuitry that outputs various data to external circuitry. For example, using the output circuitry, the scale outputs user data to external circuitry, such as a smartphone, a smartwatch, a tablet, an external server and/or processor, and/or other circuitry and devices. Scales, in various embodiments, communicate with external circuitry for various processing of user data. The external circuitry pools user data and identifies potential correlations or patterns of risks for conditions or diseases of users. The user data, however, includes various user-sensitive data and/or data that is subject to various government regulations, such as Food and Drug Administration (FDA) regulations and HIPA disclosure requirements. To securely communicate the data, the scale removes portions of the scale-obtained data that identifies the user and adds an identifier to the scale-obtained data to identify that the user-data corresponds to one user. The identifier includes code(s) that uniquely identify the user and the scale. The scale, optionally, secures the user data by encrypting all and/or portions of the user data, such as the identifier. In some embodiments, the identifier includes an alias ID. The scale outputs the secure data to the external circuitry. The scale can separately communicate identification of which scale corresponds to the respective alias ID and/or uses the same alias ID each time the scale communicates data corresponding to the user to the external circuitry. Alternatively and/or in addition, the identifier is encrypted that identifies the user and the scale, and the external circuitry replaces the identifier with an alias ID. The external circuitry stores the user data with the alias ID in a first database and stores the identification of the scale and user that corresponds to the alias ID in a second database. In this manner, the user data stored in the first database does not identify the user. Further, by storing the user data in a separate database from the identification of the alias ID and scale/user, preferably at a separate location, the pooled user data has a lower risk of being inappropriately accessed such that an external entity and/or source, such as a security hacker, identifies the respective user corresponding to the user data.

Furthermore, in various embodiments, user data from a plurality of different scales is combined to identify potential risks for conditions. For example, a plurality of users may use different scales and the user data is combined in a user-specific knowledge database. The external circuitry compares the user data within the user-specific knowledge database to determine various correlations and patterns. The user-specific knowledge database, in various embodiments, is dynamically updated overtime as more information is learned from different users. For example, the user-specific knowledge database stores data collected from a plurality of users. A first user is known to have a heart condition and has various parameters that are measured and correlated to symptoms of the heart condition. A second user is not known to have the same heart condition but has similar parameter values as the first user. The external circuitry uses the information of the first user to determine or review a potential risk for the condition for the second user. Furthermore, if the second user is subsequently diagnosed with a different (or same) heart disease than the first user, the user-specific knowledge database is updated with this information. Thereby, the user-specific knowledge database is updated with potential risk factors and parameter values associated with a condition in response to additional information from users of the scales.

In various embodiments, the external circuitry groups respective sets of user data into social groups. The social groups are based on demographics, user goals, symptoms, physiological parameter values, diagnosis, prescription drug usage, lifestyle habits, medical history, family medical history, and a combination thereof. In a specific embodiment, the external circuitry groups user data based on fitness goals (current or historical), demographic information, and scale-obtained data. The correlation, in some instances, is provided to the user, without identifying specific other users, such that the user identifies how other users of a similar demographic reached their fitness goals. In other embodiments, the correlation includes users with a specific condition, disorder, and/or disease and causes of improvements or potential lack of improvement of symptoms of the condition, disorder and/or disease, such as lifestyle changes, prescription drugs, and/or change in exercise habits or geographic location. Thereby, the pooled user data is used to educate users based on other user's successes, failures, and/or general results.

In various embodiments, in response to the social group, the external circuitry outputs a prompt that notifies the respective user of the available of a social group and generates a way to access the social group, such as a generated new blog, form, and/or page of a social network. The blog, forum and/or social network page may be semi-private in that only the users that are identified are provided access. The external circuitry provides an output to the respective scales of the identified users to invite them to the social group. In various embodiments, the scale displays the invitation using a user display and/or outputs the invitation to another user device. The invitation includes, in some embodiments, a direct link to the blog, forum, and/or webpage. Further, the prompt and/or invitation can include a display of how the user's data appears in the social group (e.g., the normalized user data) and an indication that the user can adjust the normalization data. The user accesses the social group through the scale and/or another user device. When using the blog, forum, and/or social network, the identity of the user remains anonymous. Further, the various users are given access to the social group to discuss potential successes or failures related to the correlation. For example, the user may discuss causes of improvements or potential lack of improvement of symptoms of the condition, disorder and/or disease, such as lifestyle changes, prescription drugs, and/or change in exercise habits or geographic location.

The generated blog, forum, and/or webpage includes an identification of the correlation between the users and a location for the users to communicate with one another, such as a post board. The external circuitry provides an output to the respective scales of the identified users to invite them to the page of the social network. In various embodiments, the scale displays the invitation using a user display and/or outputs the invitation to another user device. The invitation includes, in some embodiments, a direct link to the blog, forum, and/or webpage. The user accesses the social group through the scale and/or another user device.

In various embodiments, the information input by the users on the blog, forum, and/or webpage is used by the external circuitry to update the user-specific knowledge database and/or various risks for conditions. For example, the user-specific knowledge database is used to identify potential risks for conditions. A plurality of users may use different scales and the user data is combined in a user-specific knowledge database. The external circuitry compares the user data to the user-specific knowledge database, which includes other user's user data and conditions they have, to determine the risk. The user-specific knowledge database, in various embodiments, is dynamically updated overtime as more information is learned from different users, such as using the blog, forum, and/or webpage as a feedback to the database. For example, the user-specific knowledge database stores data collected from a plurality of users. A first user is known to have a heart condition and has various parameters that are measured and correlated to symptoms of the heart condition. A second user is not known to have the same heart condition but has similar parameter values as the first user. Both the first user and the second user access the single social group to discuss their heart conditions and/or symptoms. During the process, different symptoms are identified and/or the second user is diagnosed with a condition. The external circuitry uses the information to update the user-specific knowledge database. Thereby, the user-specific knowledge database is updated with potential risk factors and parameter values associated with a condition in response to additional information from users of the scales and through communication with the social network.

In accordance with various embodiments, the user data is based on sensing, detection, and quantification of at least two simultaneously acquired impedance-based signals. The simultaneously acquired impedance-based signals are associated with quasi-periodic electro-mechanical cardiovascular functions, and simultaneous cardiovascular signals measured by the impedance sensors, due to the beating of an individual's heart, where the measured signals are used to determine at least one cardiovascular related characteristic of the user for determining the heart activity, health, or abnormality associated with the user's cardiovascular system. The sensors can be embedded in a user platform, such as a weighing scale-based platform, where the user stands stationary on the platform, with the user's feet in contact with the platform, where the impedance measurements are obtained where the user is standing with bare feet.

In certain embodiments, the plurality of impedance-measurement signals includes at least two impedance-measurement signals between the one foot and the other location. Further, in certain embodiments, a signal is obtained, based on the timing reference, which is indicative of synchronous information and that corresponds to information in a BCG. Additionally, the methods can include conveying modulated current between selected ones of the electrodes. The plurality of impedance-measurement signals may, for example, be carried out in response to current conveyed between selected ones of the electrodes. Additionally, the methods, consistent with various aspects of the present disclosure, include a step of providing an IPG measurement within the one foot. Additionally, in certain embodiments, the two electrodes contacting one foot of the user are configured in an inter-digitated pattern of positions over a base unit that contains circuitry communicatively coupled to the inter-digitated pattern. The circuitry uses the inter-digitated pattern of positions for the step of determining a plurality of pulse characteristic signals based on the plurality of impedance-measurement signals, and for providing an IPG measurement within the one foot. As discussed further herein, and further described in U.S. patent application Ser. No. 14/338,266 filed on Oct. 7, 2015, which is herein fully incorporated by reference for its specific teaching of inter-digitated pattern and general teaching of sensor circuitry, the circuitry can obtain the physiological data in a number of manners.

In medical (and security) applications, for example, the impedance measurements obtained from the plurality of integrated electrodes can then be used to provide various cardio-related information that is user-specific including, as non-limiting examples, synchronous information obtained from the user and that corresponds to information in a ballistocardiogram (BCG) and an impedance plethysmography (IPG) measurement. By ensuring that the user, for whom such data was obtained, matches other bio-metric data as obtained concurrently for the same user, medical (and security) personnel can then assess, diagnose and/or identify with high degrees of confidence and accuracy.

In a number of a specific embodiments, the user stands on the scale. The scale collects signals using the data-procurement circuitry, and generates user data. The scale is used by multiple users, such as family members in a home. The scale stores user profiles for the multiple users and identifies the respective user by verifying a scale-based biometric of the user using signals collected by the scale and comparing the signals to a user profile. Further, the scale removes portions of the user data that identifies the user, adds an identifier indicative of the user and the scale to the user data, optionally encrypts at least a portion of the user data, and outputs at least a portion of the user data to external circuitry. The external circuitry processes the collected user data by replacing the identifier with an alias ID, storing the user data with the alias ID in a first database that has pooled user data from a plurality of scales, and storing identification of the respective scale and user that corresponds to the alias ID in a second database.

The external circuitry analyzes the pooled user data from the plurality of scales to identify various correlations and dynamically updates the first database over time. Based on the correlated user data sets, the external circuitry identifies various user data sets with correlation and provides users of the correlated data sets access to a social group, such as a forum, blog, and/or a webpage on a social network that identifies the correlation of the users and is accessible by the users associated with the correlated user data sets. The external circuitry outputs an indication to the scale and/or another user device regarding the available social group. The access to the social group includes selective access to normalize user data from the user data sets. The external circuitry can identify and normalize user data from the user data sets of the subset of users with the identified correlation based on prioritization data and normalization data (e.g., values). The user accesses the social group and communicates with other users regarding the correlation, such as symptoms, treatments, physician used, prescription medication used, weight loss programs, exercise habits, etc. Further, various reports are generated and displayed to the social group that indicates progress, others successes and failures, new diagnosis information or treatments, and other data. The external circuitry uses the user inputs to the forum, blogs, and/or webpage to update the user-specific knowledge database. Thereby, users with similar issues and/or goals are grouped together for communication based on scale-obtained data. The user can assist other users in identifying diagnosis and/or symptoms, in identifying successful treatment and/or ways to reduce symptoms and/or ways to obtain goals. The inputs update the first database overtime such that users are dynamically grouped and regrouped.

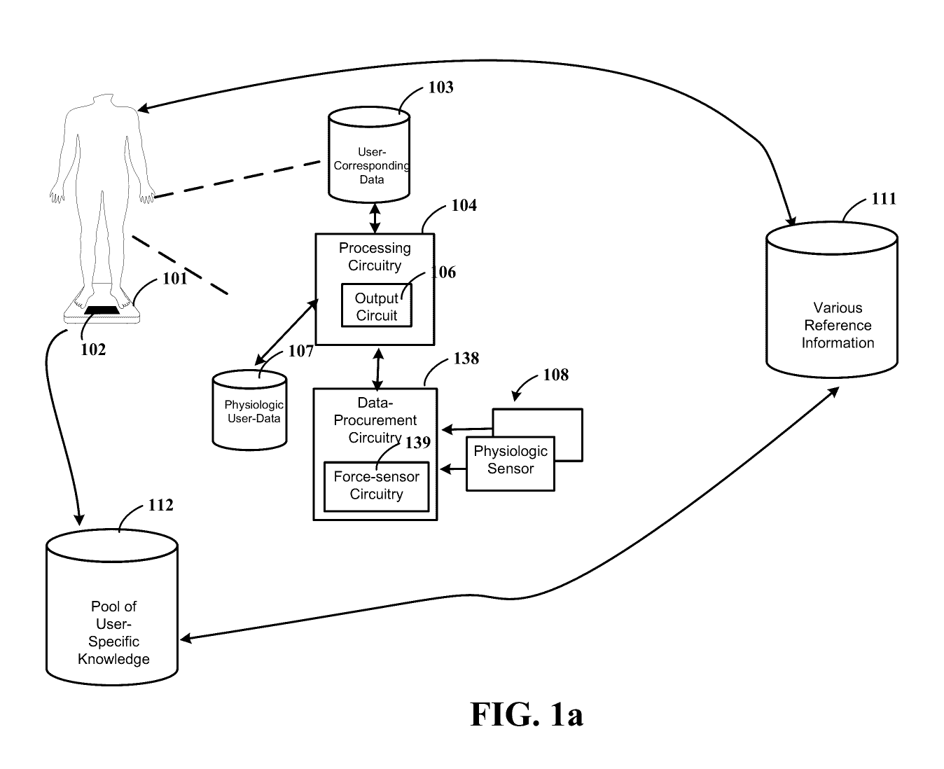

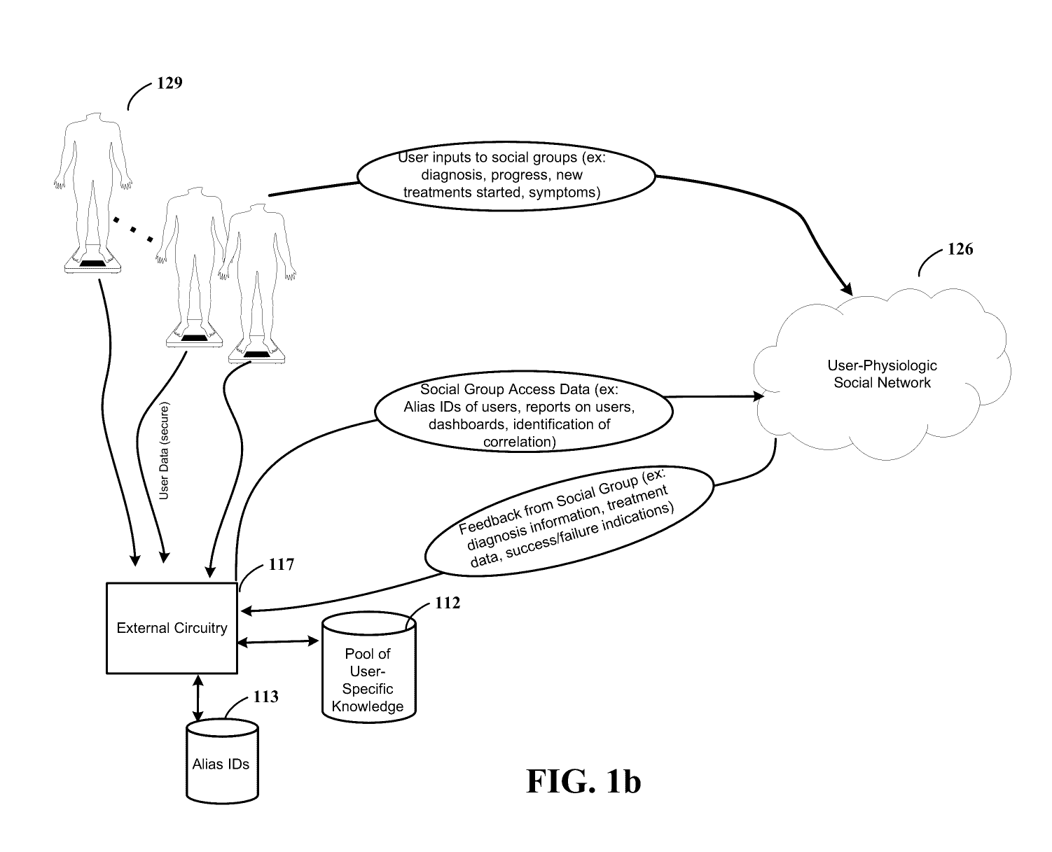

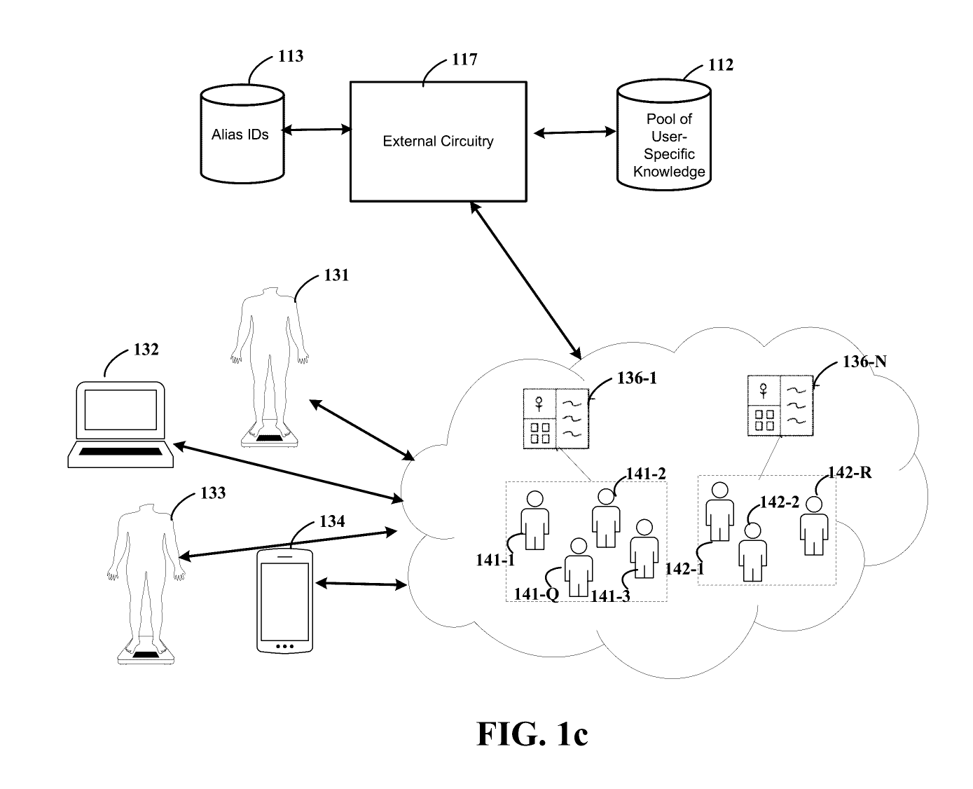

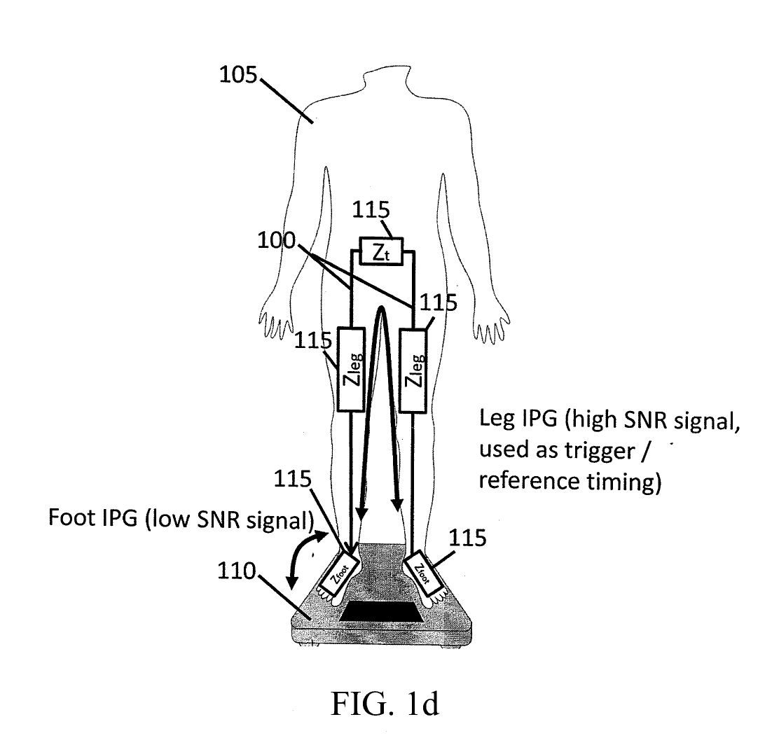

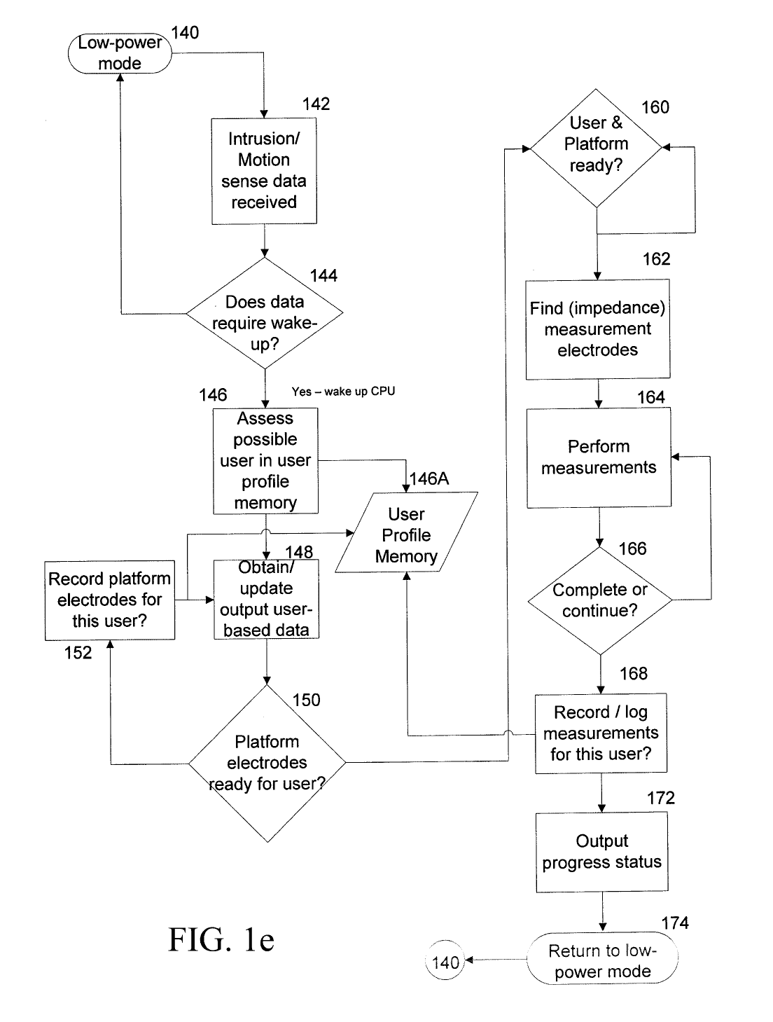

Turning now to the figures, FIG. 1a shows a scale-based user-physiologic system consistent with aspects of the present disclosure. The system includes one or more scales and user-specific knowledge database 112. In various embodiments, the system optionally includes reference information 111. The scale collects user data that is indicative of cardio-related measurements and outputs the user data to external circuitry. The external circuitry includes the reference information 111 and/or the user-specific knowledge database 112 and/or is in communication with the same. The one or more scales secure the user data for communication by removing data that identifies the user from the user data, adding an identifier that is indicative of the identity of the scale and the user to the user data, and optionally encrypting portions of the user data, such as the identifier. The external circuitry, in various embodiments, replaces the identifier with an alias ID that is independent of the identifier, stores the user data in the user-specific knowledge database 112 (e.g., a first database) and stores identification of which scale corresponds to the respective alias ID in another database (e.g., a second database). In various embodiments, the scale-based user-physiologic system is used to group users into social groups and provide the users with access to the social groups, such as a forum, blog, and/or webpage and/or provides reports regarding the social group.

Each scale includes a platform 101 and a user display 102. The user, as illustrated by FIG. 1a is standing on the platform 101 of the apparatus. The user display 102 is arranged with the platform 101. As illustrated by the dotted lines of FIG. 1a, the scale includes processing circuitry 104, data-procurement circuitry 138, and physiologic sensors 108. That is, the dotted lines illustrate a closer view of components of an example scale. In various embodiments, the user display 102 includes a foot-controlled user interface (FUI), as described in further detail herein. A FUI includes or refers to a user interface that receives inputs from the user's foot (e.g., via the platform) to allow the user to interact with the scale. A user interface includes or refers to interactive components of a device (e.g., the scale) and circuitry configured to allow interaction of a user with the scale (e.g., hardware input/output components, such as a screen, speaker components, keyboard, touchscreen, etc., and circuitry to process the inputs). The user interaction includes the user moving their foot relative to the FUI, the user contacting a specific portion of the user display, the user shifting their weight, etc. Example GUIs include input/output devices, such as display screens, touch screens, microphones, etc.

The physiologic sensors 108, in various embodiments, include a plurality of electrodes integrated with the platform 101. The electrodes and corresponding force-sensor circuitry 139 are configured to engage the user with electrical signals and to collect signals indicative of the user's identity and cardio-physiological measurements while the user is standing on the platform 101. For example, the signals are indicative of physiological parameters of the user and/or are indicative of or include physiologic data, such as data indicative of a BCG or ECG and/or actual body weight or heart rate data, among other data. As discussed further below, the signals can be force signals. The user display 102 is arranged with the platform 101 and the electrodes to output user-specific information for the user while the user is standing on the platform 101. The processing circuitry 104 includes CPU and a memory circuit with user-corresponding data 103 stored in the memory circuit. The processing circuitry 104 is arranged under the platform 101 upon which the user stands, and is electrically integrated with the force-sensor circuitry 139 and the plurality of electrodes (e.g., the physiologic sensors 108). The data indicative of the identity of the user includes, in various embodiments, user-corresponding data, biometric data obtained using the electrodes and/or force sensor circuitry, voice recognition data, images of the user, input from a user's device, and/or a combination thereof and as discussed in further detail herein.

The user-corresponding data includes information about the user (that is or is not obtained using the physiologic sensors 108,) such as demographic information or historical information. Example user-corresponding data includes height, gender, age, ethnicity, exercise habits, eating habits, cholesterol levels, previous health conditions or treatments, family medical history, and/or a historical record of variations in one or more of the listed data. The user-corresponding data is obtained directly from the user (e.g., the user inputs to the scale) and/or from another circuit (e.g., a smart device, such a cellular telephone, smart watch and/or fitness device, cloud system, etc.). The user-corresponding data 103 is input and/or received prior to the user standing on the scale and/or in response to.

In various embodiments, the processing circuitry 104 is electrically integrated with the force-sensor circuitry 139 and the plurality of electrodes and configured to process data obtained by the data-procurement circuitry 138 while the user is standing on the platform 101. The processing circuitry 104, for example, generates cardio-related physiologic data corresponding to the collected signals and that is manifested as user data. Further, the processing circuitry 104 generates data indicative of the identity of the user, such as a user ID and/or other user identification metadata. The user ID is, for example, in response to confirming identification of the user using the collected signals indicative of the user's identity.

The user data, in some embodiments, includes the raw signals, bodyweight, body mass index, heart rate, body-fat percentage, cardiovascular age, balance, tremors, among other non-regulated physiologic data. In various embodiments, the processing circuitry 104, with the user display 102, displays at least a portion of the user data to the user. For example, user data that is not-regulated is displayed to the user, such as user weight. Alternatively and/or in addition, the user data is stored. For example, the user data is stored on the memory circuit of the processing circuitry (e.g., such as the physiological user-data database 107 illustrated by FIG. 1a). The processing circuitry 104, in various embodiments, correlates the collected user-data (e.g., physiologic user-data) with user-corresponding data, such as storing identification metadata that identifies the user with the respective data.

For example, in specific embodiments, in response to the user standing on the scale, the scale collects signals indicative of cardio-physiological measurements (e.g., force signals). The processing circuitry 104, processes the signals to generate cardio-related physiologic data manifested as user data and outputs the user data to the external circuitry. In various embodiments, the processing includes adding (and later storing) data with a time stamp indicating a time at about when the physiologic parameter data is obtained.

In a number of embodiments, the processing circuitry 104 and/or the scale includes an output circuit 106. The output circuit 106 receives the user data and, in response, sends the user data, including the data indicative of the user's identity and the generated cardio-related physiologic data, from the scale for reception at a remote location (e.g., to external circuitry for assessment). In various embodiments, the output circuit 106 displays on the user display 102, the user's weight and the data indicative of the user's identity and/or the generated cardio-related physiologic data corresponding to the collected signals. The external circuitry is at a remote location from the scale and is not integrated with the scale. The communication, in various embodiments, includes a wireless communication and/or utilizes a cloud system.

In various embodiments, the external circuitry is part of a scale-based physiological social grouping system. In such embodiments, the external circuitry pools user data from a plurality of scales in a user-specific knowledge database 112. As previously discussed, the user data includes data that is user-sensitive and/or that the user would otherwise not want compromised. To prevent the data from being compromised and/or the identity of the user being learned, the processing circuitry 104 of the scale removes portions of the user data that identifies the user (e.g., user identification information) and adds an identifier (e.g., code) that uniquely identifies the scale and the user. The removed portions, in some embodiments, includes a user ID, user name, date of birth, location, and a combination thereof. The identifier, in various embodiments, includes a scale ID and a user ID. Alternatively, the identifier includes an alias ID, as discussed further herein. For example, the scale ID remains the same for each user of the scale and identifies the scale. The user ID, by contrast, is different for each user of the scale and identifies the respective user profile corresponding to the scale. The identifiers (scale ID or user ID), in some embodiments, includes numeric and/or alphabetic assignment and/or is based on identifying data, such as an Internet Protocol (IP) address of the scale and/or a social security number (or part thereof) of the user.

The external circuitry receives the user data and, in response, replaces the identifier with an alias ID. For example, the external circuitry creates an alias ID corresponding to each identifier and, for certain types of access requests, provides the alias ID in place of the identifier. Further, the external circuitry stores the user data with the alias ID in the user-specific knowledge database 112 and stores identification of the scale and user that correspond to the alias ID in another database. For security purposes, the alias ID is encrypted and access to the encrypted alias ID can be restricted. The scale and/or the external circuitry, in various embodiments, encrypt the identifier and/or the alias ID. In various embodiments, the user data is sent over time. Thereby, the first database includes historical data for the user. The alias ID, in some embodiments, is associated with a generic user profile such that user data with the alias ID is associated with the same generic user profile over time.

An alias ID, as used herein, is data that is independent of the identifier (e.g., not invertible back to the identifier). In some embodiments, the alias ID is formatted as the identifier is. That is, the alias ID is used in place of the identifier that identifies the user and the scale and that appears in the same format. Further, the alias ID includes a substitute value for the identifier that has no algorithmic relationship with the identifier and is not reversible. The alias ID is provided in place of the identifier for certain types of access requests. Therefore, the alias ID is used in place of the identifier for accessing the user data unless the user data is requested by an authorized user (such as, the user corresponding to the user data and/or a physician for a fee). The system stores the user data in the user-specific knowledge database 112 with the alias IDs, and stores an association of each alias ID to a scale and user in another database. The system may maintain the association between the alias ID and the user data, regardless of the form of the sensitive user data. Thus, the association remains the same whether the user data is decrypted, formatted, encrypted or re-encrypted using a different encryption scheme.

An output of the system provides the alias ID in place of the identifier for accesses to the user data unless the sensitive data is specifically requested by an authorized user. The alias IDs are independent of the sensitive user data in that the identifier that indicates identification of the user and the scale cannot be derived directly from the alias IDs. This independence can be implemented using a variety of alias ID creation techniques such as a randomly generated identifier, a sequentially generated identifier, or a non-invertible derivation of the transaction card identifier. The aliases may also be uniquely associated with exactly one scale and one user. In some instances, the user, administrator, or another application using the invention may configure the format of the alias IDs. For example, the user may designate that the alias IDs should be formatted to each contain six capital letters or to each contain nine digits. In another embodiment, the user may designate a portion of the identifier that is retained and used as a portion of the alias ID. In one such example, the system uses the first number of an identifier as the first number of its corresponding alias.

In various embodiments, the other database is used to identify the scale and user. For example, the external circuitry uses the other database to identify the scale and user corresponding to the alias ID. The identification, in some instances, is to provide a notification and/or additional data to the user through the scale. For example, in various embodiments, the user-specific knowledge database 112 is used to identify correlated user data and identify various patterns of risks or conditions or diseases based on the correlation. The user, in some embodiments, is notified of a potential correlation. The notification is displayed on the user display of the scale and/or another user device. In some embodiments, the external circuitry outputs the correlations that includes user data with alias IDs. For example, output data may not identify that the user has such a problem or correlation but rather generic correlations of user data with alias IDs. The output data, optionally, identifies patterns of risk for conditions or diseases based on the correlation (without actually identifying the user which has the condition or disease but indicating correlation). Further, based on the correlation, the user can receive an advertisement, such as an advertisement for a physician, prescription drug, health program, and/or social network group, as discussed further herein.

In various embodiments, the external circuitry uses the user-specific knowledge database 112 to identify users with correlations. The correlation, in some embodiments, includes patterns and/or trends, risks, and/or parameter values associated with and/or indicative of particular conditions that are common between different users. For example, the external circuitry identifies other users that have correlated user data and identify patterns of risks for conditions or diseases based on the correlation. Identifying correlated user data, for instance, includes grouping respective sets of user data into groups based on various criteria. The criteria includes symptoms, physiological parameter values, diagnosis, prescription drug usage, lifestyle habits, medical history, family medical history, and a combination thereof.

Based on the correlated user data sets, the external circuitry in some embodiments groups the users into a social group and generates a forum, blog, and/or webpage for the users of the social group to access. The users are notified of the availability of a social group via a prompt on a FUI of the scale the next time the user stands on the scale (and the scale recognizes the user using a scale-obtained biometric) and/or on a user interface of another user device. The prompt includes an indication that a social group is available. In various embodiments, the prompt provides a direct link to the generated forum, blog and/or webpage. The user accesses the social group by clicking the direct link on the FUI of the scale and/or on the user interface of another user device. Further, the identity of the users remains anonymous and/or the user can select a code-name to use. A user interface includes or refers to interactive components of a device (e.g., the scale) and circuitry configured to allow interaction of a user with the scale (e.g., hardware input/output components, such as a screen, speaker components, keyboard, touchscreen, etc., and circuitry to process the inputs). A user display includes an output surface (e.g., screen) that shows text and/or graphical images as an output from a device to a user (e.g., cathode ray tube, liquid crystal display, light-emitting diode, gas plasma, touch screens, etc.).

In various embodiments, the forum, blog, and/or webpage includes reports and/or dashboards automatically generated and displayed by the external circuitry using the user-specific knowledge database 112. The reports and/or dashboards include rankings of the user based on scale-obtained data, progress (e.g., increases or decreases in physiological parameters, weight, etc.), diagnoses, symptoms, treatments receiving, and other scale-obtained data. For example, a progress report includes increases and/or decreases in physiological parameters and/or weight of the users of the social group and identifies potential causes of the increase or decrease (e.g., correlations based on scale-obtained data and/or data from other user devices).

The external circuitry, for example, can identify and normalize user data from the user data sets of the subset of users (with the identified correlation) based on prioritization data and normalization data. The access to the social group includes selective access to the normalized user data. Normalized user data includes portions of user data from the user data sets selected using the priority data that is normalized using the normalization data. The prioritization data and/or normalization data can be default values and/or based on user input. For example, the user can provide inputs to the scale to adjust a priority level, indicate to not display particular data, adjust a sensitivity level, and/or adjust normalization values. When the user verifies an interest in participating in the social group, the user can view how their normalized user data is seen by others in the social group, such as via a FUI of the scale and/or a GUI of another device.

Prioritization data includes or refers to a prioritization of different categories of user data, including but not limited to scale-obtained physiological data, demographic data, lifestyle data (e.g., user habits include eating, drinking, smoking, sleeping, exercise, prescription medication, etc.), and diagnosis data. The categories of data can include data of different sensitivity and/or specificity levels. For example, the prioritization data can include numerical values (e.g., 1-10), binary indicators (e.g., include in social groups or not, or priority or not), and/or other ways to differentiate or group the different categories of the user data. In some specific aspects, the prioritization data can be specific to the correlation identified, and thus, a specific categories of user data may have different priorities for different uses. Particular user data may be relevant (e.g., be a risk, a symptom, a way to reduce a risk or symptom, a way to improve a goal or impact a goal) to a particular correlation. As a specific example, exercise habits and age can be relevant to arterial stiffness or declining arterial compliance as they can impact the risk for the health condition and/or improvements in the risk (e.g., lower risk). The user may adjust all or portions of the prioritization data. For example, the user may select different sensitivity or specificity values, which can impact the prioritization data and/or set the prioritization data.

The normalization data includes or refers to a numerical value or other privacy value to different categories of data. For example, the normalization data can include numerical values to normalize particular user data to and/or normalization of privacy of different categories of user data. In specific examples, user data that is provided to sets of users in a social group is normalized for privacy purposes and for sensitivity for the user. Specific users may not want their identity shown and/or may be sensitive to displaying values (such as weight or diagnosis) to different users. To protect the user's privacy and ease their comfort in using social groups, the data that users are provided access to is normalized. The normalization values can include default values and/or can be adjusted based on user input. For example, the user can view how their data is displayed in the social group prior to providing other users with access and can further adjust the normalization. Particular user data can adjusted to a numerical scale (e.g., 1-10) and/or not all of the data is displayed (e.g., don't show that the user is diagnosed with a condition). In other aspects, the user data can be normalized in other ways and/or in combination. For example, instead of displaying a user's weight, the social group is provided access to a scaled version (e.g., 1-10 or 1-100) and a percentage change in the user's weight over a period of time. As another example, instead of displaying the specific diagnosis of the users (e.g., AFIB), the user is indicated as having an arrhythmia condition. The user can adjust the data displayed to the social group overtime, such as when the user becomes more comfortable with the social group. When the particular user accesses the user data in the social group, other user's data is normalized based on the particular user's selection and/or based on the other users' selections.

In various specific examples, the normalized user data can include numerical values to indicate a general value (e.g., high or low) for the user data. As a specific example, the normalized user data can include body fat (or body-mass-index (bmi)) on a range of 1-100, with 1 being a low value and 100 being a high value, and a display of a percentage change in body fat in a period of time. Normalized user data includes or refers to user data, including scale-obtained physiological data and optionally other data (e.g., demographic or lifestyle data) that is normalized using the privacy data and normalization data. The range can, for instance, be based on average values for similar demographic users (e.g., high, medium, and low body fat values for a user of the same or demographic similar sex and age). As another example, normalized user data can include resting heartrate on a range of 1-10 or 1-100, and, optionally, include an indication of improvement in resting heartrate such as a percent change in a period of time. In other embodiments, the normalized user data can include actual weight values or weight on a range (e.g., a scaled ranged) (of 1-10 or 1-100) with no user identification and with a percent change in a period of time. In related embodiments, the normalized user data can include an amount of exercise or types of exercise in a period of time. The amount can be normalized within a range or scale of numerical values, such as 1-10 and 1-100, with one being no exercise and with 10 or 100 including a recommended (or above a recommended) amount of exercise. Further, the amount of exercise can include a number of exercise sessions in the period of time, the total time exercise, and/or an amount of time per exercise sessions. In other specific and related embodiments, the amount of exercise can include a number of steps per week that is scaled in a range (1-10 or 1-100) with the highest value (10 or 100) of the range including the user reaching above a goal. Other normalized user data can include a number of times the user stands on the scale and/or a number of times the user performs an exercise test using the scale (e.g., the scale instructs the user to exercises and identifies recovery parameters). The number of times can be presented as actual values (e.g., 10 times this week) or normalized (e.g., 1-10 or 1-100). The various user data can include weight-relevant parameters and can include various combinations as described through the present disclosure. Examples include BCG, ejection rate or indications thereof, variability in heart beats, and arrhythmia conditions or indicators.

As another specific example, loss of muscle mass and function, whether the cause is age-related sarcopenia or otherwise, can be assessed with weight changes and other user-specific physiologic indicators, including but not limited to PWV (pulse wave velocity) and body-fat and diet measurements/changes that are suggestive/indicative of abnormalities often associated with aging and/or related causes of physiologic declinations. In various embodiments, the scale identifies that the user has reached middle age and has one or more other factors for declining arterial compliance and/or muscle-loss issues. The scale, in response, provides the user with articles, journals, and access to a social group to motivate and psychologically influence the user to change particular lifestyle habits to mitigate or prevent such issues which would otherwise evince (e.g., arterial/muscle) compliance declines. The normalized user data for the social group can include body fat, weight, and/or PWV (as normalized on a scaled range of 1-10 or 1-100) and percent change over time. Further, the normalized user data can include identification of changes in diet and correlation with the change in body fat, weight, and/or PWV. The change in diet can be normalized to include general (and not specific detail) detail, such as changes or values of calorie, fat, and sugar intake.

In connection with the above-described embodiments and other embodiments described herein, the system incorporates communication circuitry that can vary. For example, the scale includes communication circuitry, external circuitry includes communication circuitry and/or a user device includes communication circuitry. The external circuitry can include a server or standalone CPU with communication circuitry, among other circuitry. The user device can be a smart device having communication circuitry. A smart device is an electronic device, generally connected to other devices or networks via different wireless protocols such as Bluetooth, NFC, Wi-Fi, 3G, etc., that can operate to some extent interactively and autonomously. The smart device can include communication circuitry and GUI, such as keyboards and touchscreens which are controlled by circuitry typically programmed for the smart device. Examples include a tablet, a smartphone, a smartwatch, a laptop computer, etc.

In various embodiments, the scales act as hubs for sensitive-user data and collects sensitive-user data from a plurality of user devices. The user devices include devices such as smartphones, smartwatches or fitness watches, exercise tracking devices, heart monitors, smartbeds, among other devices. The user device includes processing circuitry and, optionally sensor circuitry, configured to collect data from the user. The correlations, in such embodiments, are based on data from user devices, data from the scale, and/or a combination thereof. The scale aggregates the data from the various devices and outputs the data in response to a scale-based biometric.

In accordance with various embodiments, the scale authorizes communication of user data and/or differentiates between two or more users using a scale-based biometric. In some embodiments, the scale uses a cardiogram of the user and/or other scale-obtained biometrics to differentiate between two or more users. The scale-obtained data includes health data that is user-sensitive, such that unintentional disclosure of scale-obtained data is not desired. Differentiating between the two or more users and automatically communicating (e.g., without further user input) user data responsive to scale-obtained biometrics, in various embodiments, provides a user-friendly and simple way to communicate data from a scale while avoiding and/or mitigating unintentional (and/or without user consent) communication. For example, the scale, such as during an initialization mode for each of the two or more users, collects user data to identify the scale-based biometrics and stores an indication of the scale-based biometrics in a user profile corresponding with the respective user. During subsequent measurements, the scale recognizes the particular user by comparing collected signals to the indication of the scale-based biometrics in the user profile. The scale, for example, compares the collected signals to each user profile of the two or more users and identifies a match between the collected signals and the indication of the scale-based biometrics. A match, in various embodiments, is within a range of values of the indication stored. Further, in response to verifying the scale-based biometric(s), a particular communication mode is authorized.

In accordance with a number of embodiments, the scale identifies one or more of the multiple users of the scale that have priority user data. The user data with a priority, as used herein, includes an importance of the user and/or the user data. In various embodiments, the importance of the user is based on parameter values identified and/or user goals, such as the user is an athlete and/or is using the scale to assist in training for an event (e.g., marathon) or is using the scale for other user goals (e.g., a weight loss program). Further, the importance of the user data is based on parameter values and/or user input data indicating a diagnosis of a condition or disease and/or a risk of the user having the condition or disease based on the scale-obtained data. In some embodiments, user(s) with cardio-related physiologic data with threshold priority have data sent to the external circuitry for various purposes, include forming social groups. In specific embodiments, the priority is based on prioritization data (as previously described).

For example, the scale-obtained data of a first user indicates that the user is overweight, recently had an increase in weight, and has a risk of having atrial fibrillation (e.g., potentially has a medical issues). The first user is identified as a user corresponding with priority user data. A second user of the scale has scale-obtained data indicating an increase in recovery parameters (e.g., time to return to baseline parameters) and the user inputs an indication that they are training for a marathon. The second user is also identified as a user corresponding with priority user data. The scale displays indications to the user with the priority user data, in some embodiments, on how to use the scale to communicate the user data to external circuitry for further processing, correlation, and/or other features, such as social network connections. Further, the scale, in response to the priority, displays various feedback to the user, such as user-targeted advertisements and/or suggestions. In some embodiments, only users with priority user data have data output to the external circuitry to determine correlations, such as risks, although embodiments in accordance with the present disclosure are not so limited.

In some embodiments, one or more users of the scale have multiple different scale-obtained biometrics used to authorize different communication modes. The different scale-obtained biometrics are used to authorize communication of different levels of user sensitive data, such as the different user-data types and sensitivity values as illustrated in the above-table. For example, in some specific embodiments, the different scale-obtained biometrics include a high security biometric, a medium security biometric, and a low security biometric, as discussed in further detail herein.

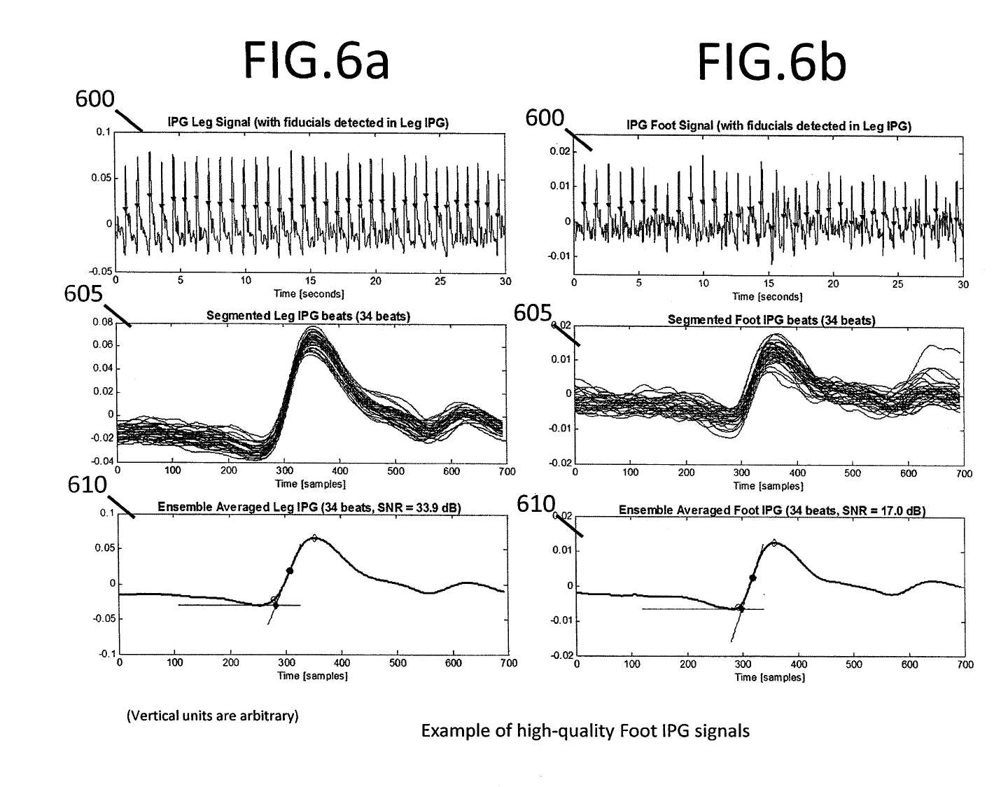

In a specific example, a low security biometric includes estimated weight (e.g., a weight range), and a toe tap on the FUI. Example medium security biometrics includes one or more of the low security biometric in addition to length and/or width of the user's foot, and/or a time of day or location of the scale. For example, as illustrated by FIGS. 6 and 18a-18c, the scale includes impedance electrodes that are interleaved and engage the feet of the user. The interleaved electrodes assist in providing measurement results that are indicative of the foot length, foot width, and type of arch. Further, a specific user, in some embodiments, may use the scale at a particular time of the day and/or authorize communication of data at the particular time of the day, which is used to verify identity of the user and authorize the communication. The location of scale, in some embodiments, is based on Global Positioning System (GPS) coordinates and/or a Wi-Fi code. For example, if the scale is moved to a new house, the Wi-Fi code used to communicate data externally from the scale changes. Example high security biometrics include one or more low security biometrics and/or medium security biometrics in addition to cardiogram characteristics and, optionally, a time of day and/or heart rate. Example cardiogram characteristics include a QRS complex, and QRS complex and P/T wave.

The social grouping, in specific embodiments, is provided as a hierarchy of service. A service, as used herein, includes a function and/or action performed using the platform system and uses and/or is in response to scale-obtained data. A hierarchy of services include different services enabled in response to user selection and activation of subscription levels. The subscription levels have different weighted values that activate the subscription level. Further, each subscription level is associated with one or more services. For example, the scale-obtained data from the particular scale drives a physiological related prompt for a service.

The weighted values of the subscription levels, in some embodiments, is based on the value of the service or corresponding data to the user, the user-sensitivity and/or regulation of the corresponding data, the value of the corresponding data to the service provider/provider of the scales, value of the corresponding data to the requester. In various embodiments, the value of the service and/or corresponding data is determined based on a level of security of the data, a level of technical detail of the data, and/or a likelihood of diagnosing the user based on the data. The requester of the data provided by the service, in various embodiments, includes a third party, such as a researcher, physician, government entity, and/or other entity. The different subscription levels have different weighted values that, in some embodiments, increase with the levels of subscription.

In a number of specific embodiments, social groupings are provided as services in a plurality of different subscription levels. For example, in a first subscription level, a user is provided access to a social group based on exercise interest and/or goals or other consumer related interest. At a second subscription level, a user is provided access to physiological social group, which is based on scale-obtained data and/or diagnosis of the scale-obtained data by a physician. At a third subscription level, a user is provided access to the (more) professional social group. For example, a physician participates in the professional social group with other users and/or actively tracks progress of the user. Alternatively and/or in addition, the physician uses the professional social group to perform a study and/or experiment.

In some embodiments, the social groups are intra scale and/or intra scale. The social grouping of an intra scale includes grouping the users of the scale and providing various reports, updates, alerts, and/or forums for the users of the group to interact. The forum, in some embodiments, includes a private (or public) page of a social network webpage that the users of the group access and communicate. A private page, for instance, is only accessible by the users of the group and/or persons authorized by users of the group. In other embodiments, the social groupings are inter scale. For example, an external circuitry, such as a server CPU, may receive user data (with user identifying data removed) from a plurality of scales and identifies various users with correlated user data. The users with correlated user data, such as demographic data and/or scale-obtained data, are grouped by the external circuitry without user input. The external circuitry outputs an indication of an available social group to the scales of the users with the correlated user data and each scale displays, using the FUI, an alert of an available social group. The user accesses the social grouping using the FUI and/or a standalone CPU that is in communication with the scale. For example, in response to an alert, the user selects an interest in the social grouping using the FUI. The scale outputs the indication and a link to a webpage or application associated with the social group (or information on how to access the social grouping) the standalone CPU, such as a user's smartphone or tablet. The webpage includes, in some embodiments, a page of a social network, an application or portal for the user to log-in to, a forum, etc. In various embodiments, data is tracked for users of the social group and reports are provided, such as rankings of the users in the group, progress of the users, new observations, and/or information learned. Alternatively and/or in addition, the users of the group are provided a forum to discuss various health issues, successes, failures, exercise, eating, etc.

As a specific example, a scale is used by a family training for a marathon. Each member of the family uses the scale to track various physiological parameters, including cardiogram related characteristics, recovery parameters, weight, body-mass-index, and exercise results. The family is grouped into an intra scale social grouping and provided with alerts when reports of progress and/or rankings are available for the family. In another specific example, multiple scales are used by different users located at different locations that have indicators for atrial fibrillation, are female, are over-weight, and are over the age of sixty-years old. The users are grouped into an inter scale social grouping and provided with an alert of an available social grouping. In response to at least a subset of the users selecting an interest in the social grouping, the subset of users are provided with a link to a webpage, portal, application, and/or forum. The subset of users access the link and are connected one another. In various embodiments, user data (with user identifying data removed) is displayed to the social group so that users can view other users' success and/or failures.

The user-specific knowledge database 112 includes pooled user data from a plurality of scales that is updated over time. Thereby, data from the scales, in some embodiments, is used to identify trends, risks, and/or parameter values associated with and/or indicative of particular conditions. In response to the update, the social groups are revised. For example, a user may have previously had access to a first social group and later does not as the correlation is removed. Further, inputs to the forums, blogs, and/or webpages are used to update the user-specific knowledge database 112.

In various embodiments, the external circuitry receives the user data and identifies a risk that the user has a condition using the reference information and the user data provided by the scale. The risk is identified by comparing the user data to the reference information (and/or the user-specific knowledge database 112) and identifying a match. The risk of a condition, as previously discussed, includes a probability that the user has the condition and a severity of the condition. Further, the risk is used to correlate the user with other users to form social groups.

In response to identifying the risk, the external circuitry derives and/or identifies and outputs generic health information correlating to the condition to the scale. The generic health information is tailored to the user based on the identified risk. As previously discussed, the generic health information includes information on risk factors for the condition, symptoms of the condition, and suggestions. The generic health information does not indicate that the user has the condition or the risk of the condition identified, in a number of embodiments.

In various embodiments, the scale and/or other user devices is used as feedback in response to the identified risk. For example, the external circuitry, in response to the identified risk, determines questions to ask the user and/or additional tests to perform and outputs the number of questions to the scale to ask the user and/or the additional tests to perform. The questions can include asking if the user has a diagnosis from a doctor, asking if the user is experiencing particular symptoms, and asking the user for family medical information. The scale, using the processing circuitry and the user display, provides the number of questions to the user (including asking if the user has a symptom occurring). The scale, using the processing circuitry and the output circuit, outputs the response to the questions to the external circuitry and the external circuitry verifies and/or adjusts the risk using the responses to the questions. For example, in various embodiments, a user may not realize they are experiencing a symptom (e.g., heart rate is raised and/or difficulty breathing). The questions ask the user about potential symptoms of the condition identified (e.g., associated with the risk) and is used to revise the risk determined. The user is provided with generic health information about the condition that may include the various symptoms to assist the user in recognizing the symptoms and discussing the same with their physician.

In a number of embodiments, the scale asks the user about diagnosis from a doctor. For example, the user may have been diagnosed with heart failure and the user can input this knowledge to the scale. The scale outputs the response to the external circuitry and the external circuitry identifies misdiagnosis information associated with the condition. For example, in some instances, when a user is diagnosed with condition Y, they actually have condition X. The external circuitry determines and outputs generic misdiagnosis information to the scale.

In other related embodiments, the external circuitry, in response to the identified risk, determines additional tests or measurements to be performed. In various embodiments, the scale is used to perform the additional test and/or other circuitry is used. For example, the external circuitry determines and outputs a test, to the scale, for the scale to perform. The scale, including the data-procurement circuitry, performs the test and outputs results to the external circuitry. Using the results, the external circuitry verifies and/or adjusts the risk. Furthermore, the user data and/or results from the test are used to update the user-specific knowledge database 112.

Although the present example embodiments provided above are in reference to external circuitry performing the determination, embodiments in accordance with the present disclosure are not so limited. For example, the processing circuitry 104 can determine the risk by accessing the reference information 111 or the feedback information while the user is standing on the platform 101.

FIG. 1b shows an example of scale-based user-physiological social grouping system comprised of a plurality of scales and external circuitry consistent with aspects of the present disclosure. The scale-based user-physiologic heuristic social grouping system includes a plurality of scales 129 and external circuitry 117. Each scale is configured to monitor signals from a plurality of users, correlate the respective data with the appropriate user using scale-based biometrics and user profiles, and communicate the signals and/or data to the external circuitry.

The external circuitry 117, in various embodiments, includes processing circuitry and a memory circuit. The external circuitry 117 receives the user data from the scale and stores the user data with an alias ID replacing identifying information in the user-specific knowledge database 112. The user data is collected and stored by the external circuitry 117 over time. For example, the external circuitry 117 validates the received user data as corresponding to a particular user associated with an alias ID based on the identifier and correlates the received user data with other user data stored in the user-specific knowledge database 112 and associated with the alias ID. The external circuitry 117 then updates the user-specific knowledge database 112 with the user data and/or other feedback data obtained. In response to not identifying the identifier (in the second database), the external circuitry 117 generates a new alias ID for the respective scale and user. Further, the external circuitry 117 stores an indication of which scale and user corresponds to the alias ID in another database 113. For example, the other database 113 includes a list of alias IDs to scale IDs and user IDs to identify the scale corresponding to the alias ID and the respective user of the scale. Alternatively and/or in addition, the scale outputs user data with an alias ID. In some embodiments, the scale outputs the correlation of the alias ID with a respective scale and user to the external circuitry 117.

As previously discussed, in some embodiments, the external circuitry 117 generates alias IDs for association with sensitive user. Typically, the alias ID is randomly generated, but it also can be generated by other means, such as a sequential generation or by generating a hash value of the sensitive data. The system then stores the alias ID and user data, which is optionally encrypted, in a user-specific knowledge database 112 and stores the correlation of the alias ID with the scale and the user in another database 113. In an example embodiment, the user of the external circuitry 117 determines the format of the alias IDs. In another embodiment, the alias IDs have the same format as the original identifier. For example, if the identifiers are sixteen digits long, the alias ID is also sixteen digit identifiers.

After the encrypted data and the alias identifier are generated, the external circuitry 117 provides access to the user data with alias ID, in various embodiments. The access, in some embodiments, includes the external circuitry 117 grouping the users into social groups based on identified correlations between user data sets and providing portions of the user data to the social group, such as a report and/or dashboard, as previously discussed. In various embodiments, the social group is accessed using the Internet 126, such as a webpage that contains a blog, forum, and/or social network page and/or an application that is accessed. The external circuitry 117 provides the user data with the alias IDs instead of the identifiers. In this manner, user data can be used without supplying the original identification of users/scales that correspond to the user data. Further, the users of the social group are anonymous and are identified by the alias IDs.

Accordingly, in various embodiments, the external circuitry 117 identifies various correlations between the user data stored in the user-specific knowledge database 112 and associated with different users. The correlation, in some embodiments, includes patterns and/or trends, risks, and/or parameter values and/or various demographic information and user goals. In various specific embodiments, the patterns and/or trends, risks, and/or parameter values are associated with and/or indicative of particular conditions. For example, the external circuitry 117 identifies other users that have correlated user data and identified patterns of risks for conditions or diseases based on the correlation. Identifying correlated user data, for instance, includes grouping respective sets of user data into groups based on various criteria. The criteria includes symptoms, physiological parameter values, diagnosis, prescription drug usage, lifestyle habits, medical history, family medical history, and a combination thereof.

In some embodiments, the external circuitry 117 includes and/is in communication with a database storing reference information. The reference information includes data and statistics of a variety of conditions, symptoms, parameters values indicative of conditions, assessment data of people experiencing the condition, government provided health information and/or databases, and a combination thereof. The reference information is stored in a structured database and/or in an unstructured database. In some embodiments, the user-specific knowledge database 112 is a portion of the reference information. The user-specific knowledge database 112 includes pooled user data from a plurality of scales 129 that is updated over time. Thereby, data from the scales, in some embodiments, is used to identify trends, risks, and/or parameter values associated with and/or indicative of particular conditions.

In various embodiments, the risks identified are used to provide generic health information to the user. For example, the external circuitry 117 identifies the scale and user that the particular user data is associated with and outputs data, such as the generic health information, to the identified scale. The external circuitry 117 identifies which scale a particular user data set corresponds to that has an identified correlation or risk using the other database 113. The identification, in some embodiments, includes identification of the scale, and, optionally, a specific user. The external circuitry 117, in various embodiments, identifies generic health information to provide the user and outputs the generic health information to the scale. The generic health information is displayed to the user, such as using the scale display or another user device depending on user preferences. For example, in response to identifying that the user is standing on the scale using a scale-based biometric, the scale displays an indication that generic health information is available to the user and/or a synopsis of the generic health information and to log-in to their smartphone or other user device to view the generic health information. The generic health information, as discussed further herein, includes various symptoms, risks factors, or advice to provide the user based on the identified correlation.

In various embodiments, the external circuitry 117 revises correlations identified using the pooled user data in the user-specific knowledge database 112 over time. For example, user data is received from the plurality of scales 129 over time. Further, additional users receive a scale and provide additional data. Over time, the scale obtains additional data from the existing users and the additional users. The external circuitry 117 dynamically revises and updates correlations of the user-specific knowledge database 112 based on the additional user data received from the plurality scales and additional scales added to the system. For example, the external circuitry 117 receives the user data and identifies a risk that the user has a condition using the user-specific knowledge database 112 and/or reference information and the user data provided by the scale. The risk is identified by comparing the user data to the reference information and pooled user data and identifying a match. The risk of a condition, as previously discussed, includes a probability that the user has the condition and a severity of the condition.

In accordance with various embodiments, although not illustrated by FIG. 1a or FIG. 1b, the system includes an additional sensor circuitry that is external to the scale. The additional sensor circuitry can include a communication circuit which is configured and arranged to engage the user with electrical signals and collect therefrom signals indicative of an ECG of the user. The sensor circuitry, which may include and/or be correlated with processing circuitry configured to derive an ECG from the collected signals. The sensor circuitry communicates the ECG to the external circuitry 117 and the scale can communicate a BCG to the external circuitry 117.

In accordance with various embodiments, the external circuitry 117 updates the user-specific knowledge database 112 using various user information. For example, the user-specific knowledge database 112 includes user data from a plurality of scales 129. The external circuitry 117 and/or the scale updates the user-specific knowledge database 112 with the user data, the test results, and the responses to the questions. Further, the user enter various information into the blog, forum, and/or webpage, which is used to update the user-specific knowledge database 112. For example, a user may indicate they are trying a new prescription medication and they are seeing increased results in physiological parameters. The external circuitry verifies this, by viewing the user's scale-obtained data. This information is used to dynamically update the user-specific knowledge database 112 and potentially revises (e.g., increase or decreases) risks identified by the external circuitry 117.

In accordance with the present disclosure, a risk for a condition is identified and/or adjusted based on demographics of the users, disorders, disease, symptoms, prescription or non-prescription drugs, treatments, past medical history, family medical history, genetics, life style (e.g., exercise habits, eating habits, work environment), among other categories and combinations thereof, and based on user data in the stored user database. The risk is provided to a scale, for example, in response to a request. A particular scale, in some embodiments, is provided the correlation using data that has alias IDs and in response to an indication that the user is interested in the data and based on scale-obtained data corresponding to the user. In a number of embodiments, various physiological factors are an indicator for a disease and/or disorder. For example, an increase in weight, along with other factors, can indicate an increased risk of atrial fibrillation. Further, atrial fibrillation is more common in men. In some instances, symptoms of a particular disorder can be different for different categories of interest (e.g., symptoms of atrial fibrillation can be different between men and women). For example, in women, systolic blood pressure is associated with atrial fibrillation. In other instances, sleep apnea may be assessed via an ECG and is correlated to weight of the user. Furthermore, various cardiac conditions can be assessed using an ECG. For example, atrial fibrillation can be characterized and/or identified in response to a user having no p-waves, no QRS complex, and no baseline/inconsistent beat fluctuations. Atrial flutter, by contrast, can be characterized by having no p-wave, variable heart rate, having QRS complexes, and a generally regular rhythm. Ventricular tachycardia (VT) can be characterized by a rate of greater than 120 beats per minute, and short or broad QRS complexes (depending on the type of VT). Atrio-Ventricular (AV) block can be characterized by PR intervals that are greater than normal (e.g., a normal range for an adult is generally 0.12 to 0.20 seconds), normal-waves, QRS complexes can be normal or prolong shaped, and the pulse can be regular (but slow at 20-40 beats per minute). For more specific and general information regarding atrial fibrillation and sleep apnea, reference is made herein to https://www.clevelandclinicmeded.com/medicalpubs/diseasemanagement/cardiology/atrial-fibrillation/ and http://circ.ahajournals.org/content/118/10/1080.full, which are fully incorporated herein for its specific and general teachings. Further, other data and demographics that are known and/or are developed can be added and used to derive the various reference information.

Such generic health information includes life-style suggestions, suggested prescription medicine and/or why it is prescribed, and/or other advice, such as symptoms that the user should watch for. For instance, the user data may suggest that the user has a heart condition and/or disorder. The generic health information suggests prescription medicine to the user to ask their physician about and/or provides potential symptoms that the user should watch for and/or should go to the physician's office or an emergency room if the symptoms arise.

In various embodiments, the system includes additional scales illustrated by FIG. 1a or 1b. For example, the external circuitry receives user data from a plurality of scales located at a variety of locations. The user data, in various embodiments, is automatically sent from the scales to the external circuitry. The external circuitry is configured to identify risk for various users using the data from the plurality of scales, output generic health information, and updated the user-specific knowledge database. In various embodiments, the external circuitry includes computer-readable instructions executed to perform the various functions.

For example, the external circuitry 117 receives the user data that corresponds to the plurality users from the plurality of scales 129. The respective user data is received at over-lapping times and/or separate times. In response to receiving the user data, the external circuitry 117, in various embodiments, identifies the respective plurality of users based on an identifier and/or other identifying data and, correlates the received user data with generic profiles of the respective plurality of users based an already generated alias ID and/or a newly generated alias ID. Each alias ID identifies that the user data corresponds to a particular user (e.g., and previously stored data corresponding to that particular user), but does not provide an identity of the user. In a number of embodiments, the external circuitry 117 identifies (e.g., determines) risks for conditions or diseases by comparing the user data with reference information. The external circuitry 117 identifies that a particular user is at risk for the condition or disease, identifies the respective user and scale using the second database, and outputs the generic health information to the scale that is tailored to each respective user based on the risk for the condition. The external circuitry 117 further instructs the scales to collect feedback data, including symptoms experiences, demographic information, medical history information etc., and uses the feedback data to revise and/or verify the risk. In some embodiments, the feedback data and the user data is used to update a user-specific knowledge database 112, which is used to refine the identified risks.

FIG. 1c illustrates an example of providing access to social groups using a scale-based user-physiologic social grouping system. The scale-based user-physiologic social grouping system includes a plurality of scales and external circuitry 117. Each scale is configured to monitor signals from a plurality of users, correlate the respective data with the appropriate user using scale-based biometrics and user profiles, and communicate the signals and/or data to the external circuitry.

As previously discussed, in some embodiments, the external circuitry 117 generates alias IDs for association with sensitive user. Typically, the alias ID is randomly generated, but it also can be generated by other means, such as a sequential generation or by generating a hash value of the sensitive data. The system then stores the alias ID and user data, which is optionally encrypted, in a user-specific knowledge database 112 and stores the correlation of the alias ID with the scale and the user in another database 113. In an example embodiment, the user of the external circuitry 117 determines the format of the alias IDs. In another embodiment, the alias IDs have the same format as the original identifier. For example, if the identifiers are sixteen digits long, the alias ID is also sixteen digit identifiers.

The external circuitry 117 identifies various correlations between the user data stored in the user-specific knowledge database 112 and associated with different users. The correlation, in some embodiments, includes patterns and/or trends, risks, and/or parameter values associated with and/or indicative of particular conditions. For example, the external circuitry 117 identifies other users that have correlated user data and identified patterns of risks for conditions or diseases based on the correlation. Identifying correlated user data, for instance, includes grouping respective sets of user data into groups based on various criteria. The criteria includes symptoms, physiological parameter values, diagnosis, prescription drug usage, lifestyle habits, medical history, family medical history, and a combination thereof.

Based on the correlations, the external circuitry groups the users into social groups. The user is provided a prompt on a user device 131, 132, 133, 134 and can access the social group using the respective device. Alternatively, the user provides an indication to display the prompt on another user device. In various embodiments, the social group is accessed using the Internet, such as a webpage 136-1, 136-N that contains a blog, forum, and/or social network page and/or an application that is accessed. Each webpage 136-1, 136-N corresponds to a respective social group and is accessed by users of the groups. The webpages illustrates various reports and dashboards indicating changes in scale-obtained data and various symptoms, treatments, exercise, and/or other information available. The external circuitry 117 automatically populates and updates the reports over time using subsequently received scale-based data.

The access to the social group can include selective access to normalized data from the user data sets of the subset of users of the social group. The external circuity can identify and normalize data form the use data sets based on prioritization data and normalization data, as previous described. Further, the prioritization data can be dependent on or a function of the specific correlation of the social group. The prioritization data and normalization data can be default values and/or can be based on inputs from the users. For example, each respective user can view their user data (as normalized and would be displayed to other users of the social group) prior to joining the social group. The user can verify and/or adjust the normalization.

In various embodiments, the webpages 136-1, 136-N are semi-private. A semi-private webpage is accessible by the user of the group and potential user invited by other users of the social group. For example, the users 141-1, 141-2, 141-2, 141-Q of the first social group have access the first webpage 136-1 not the second webpage 136-2. Similarly, the users 142-1, 142-2, 142-R of the second social group have access to the second webpage 136-N but not the first webpage 136-1.

The scale can be used by multiple different users. A subset or each of the different users can have various user devices (e.g., peripheral devices such as cellphones, smartwatches, laptop or desktop computers). The multiple users may synchronize their respective user devices to the common scale (or to multiple scales). One or more of the users can be provided with access to the social group, as previously described herein. The user is provided with the access to the social group, including portions of user data, via display on the FUI of the scale and/or an external GUI of a user device. The scale can default display to the FUI and/or the external GUI based on use of the scale. For example, the scale can be in a consumer mode, a professional mode, and/or a combination consumer/professional mode (among other modes). Data provided to the user and/or the professional can default to be displayed on the FUI of the scale, the GUI of the user device, and/or a GUI of other external circuitry depending on the use of the scale.

A consumer mode includes a scale as used and/or operated in a consumer setting, such as a dwelling. In a consumer mode, social group data can default to display on the FUI of the scale. The defaulted display of data can be revised by the user providing inputs to display the data on the GUI of the user device or a GUI of another external circuitry (e.g., a standalone CPU) and/or automatically by the scale based on past scale-based actions of the user. As a specific example, a scale is located in a dwelling with five different people. Each of the five different people use the scale, and three of the five people are provided with access to social groups. When a first user of the users that is provided access to a social group stands on the scale, the scale recognizes the first user and displays an indication of available social group data on the FUI of the scale. The defaulted display is adjusted by the first user overtime and/or at the time by the user providing inputs to the scale. For example, the first user provides a user input to the scale to display data on the GUI of the user device multiple times (e.g., more than a threshold number of times, such as five times). In response, the scale adjusts the defaulted display of social group data for the first users and subsequently (or at the time) outputs data to the GUI of the user device. The display on the FUI of the scale and/or GUI of the user device (or other external circuitry) can include an indication of available social group data, a preview of the portion of the user's user data to be displayed to other users in the social group, and/or an option to override the display of data, among other displays.

In other instances the scale is used in a professional setting, such as a medical office, and/or in a professional mode. A professional mode includes an operation of the scale as used and/or operated in a professional setting, such as a doctor's office, exercise facility, nursing home, etc. In a professional mode, the scale is used by different users that may not be familiar with one another. The different users may have user devices and/or services with the professional to track and/or aggregate data from the respective user device. As a specific professional mode example, a scale is located at a doctor's office and is used to obtain data from multiple patients (e.g., 10 in a day, 500 in a year). When a patient checks-in, they stand on the scale and the scale-obtained data is output to external circuitry for document retention and/or other purposes. A subset (or all) of the patients have activated a service with doctor that corresponds with and/or includes acquisition and/or aggregation of data from a user device. For example, a user with AFIB can wear a smartwatch to track various cardio-related data during exercise and/or other periods of time and which is output to the scale at the doctor's office and/or other external circuitry.

In a professional mode, the scale is not owned by the user and/or can be in a location that other people may see the data on the FUI of the scale (e.g., such as in an exercise facility and/or lobby of a health care profession). For privacy purposes, the display of social group data may default to the GUI of the user device. Alternatively, the display may default to the FUI of the scale to display the availability of a social group and, responsive to user verification or authority to access the social group, defaults to display on the GUI of the user device.

The scale can also be in a combination consumer/professional mode. A combination consumer/professional mode includes a scale as used and/or operated in a consumer setting for purposes and/or uses by a professional, and/or in a professional setting for purposes and/or uses by the consumer (e.g., use by the consumer outside of the professional setting and/or in addition to). As a specific example, a scale is located at a user's dwelling and used by multiple family members. A first user of the family is diagnosed with a heart-related condition and the doctor may offer a service to review data from the scale and a user device of the first user. When the other family members stand on the scale, the scale operates in the consumer display mode. The other family members may or may not have user devices and the scale operates to display data via the consumer mode. When the first user that is diagnosed with heart-related condition stands on the scale, the scale recognizes the user and operates in a professional mode or a combination display mode. For example, the scale outputs aggregated data from the scale and the user device to external circuitry that is accessible by the doctor of the first user. During the combination consumer/professional mode, portions of scale-obtained data for a particular user may default to display on external circuitry, such as a standalone or server CPU that is accessible by the professional.

The remaining figures illustrate various ways to collect the physiologic data from the user, electrode configurations, and alternative modes of the processing circuitry. For general and specific information regarding the collection of physiologic data, electrode configurations, and alternative modes, reference is made to U.S. patent application Ser. No. 14/338,266 filed on Oct. 7, 2015, which is hereby fully incorporated by references for its teachings.

FIG. 1d shows current paths 100 through the body of a user 105 standing on a scale 110 for the IPG trigger pulse and Foot IPG, consistent with various aspects of the present disclosure. Impedance measurements 115 are measured when the user 105 is standing and wearing clothing articles over the feet (e.g., socks or shoes), within the practical limitations of capacitive-based impedance sensing, with energy limits considered safe for human use. The measurements 115 can be made with non-clothing material placed between the user's bare feet and contact electrodes, such as thin films or sheets of plastic, glass, paper or wax paper, whereby the electrodes operate within energy limits considered safe for human use. The IPG measurements can be sensed in the presence of callouses on the user's feet that normally diminish the quality of the signal.

As shown in FIG. 1d, the user 105 is standing on a scale 110, where the tissues of the user's body will be modeled as a series of impedance elements, and where the time-varying impedance elements change in response to cardiovascular and non-cardiovascular movements of the user. ECG and IPG measurements sensed through the feet can be challenging to take due to small impedance signals with (1) low SNR, and because they are (2) frequently masked or distorted by other electrical activity in the body such as the muscle firings in the legs to maintain balance. The human body is unsteady while standing still, and constant changes in weight distribution occur to maintain balance. As such, cardiovascular signals that are measured with weighing scale-based sensors typically yield signals with poor SNR, such as the Foot IPG and standing BCG. Thus, such scale-based signals require a stable and high quality synchronous timing reference, to segment individual heartbeat-related signals for signal averaging to yield an averaged signal with higher SNR versus respective individual measurements.

The ECG can be used as the reference (or trigger) signal to segment a series of heartbeat-related signals measured by secondary sensors (optical, electrical, magnetic, pressure, microwave, piezo, etc.) for averaging a series of heartbeat-related signals together, to improve the SNR of the secondary measurement. The ECG has an intrinsically high SNR when measured with body-worn gel electrodes, or via dry electrodes on handgrip sensors. In contrast, the ECG has a low SNR when measured using foot electrodes while standing on said scale platforms; unless the user is standing perfectly still to eliminate electrical noises from the leg muscles firing due to body motion. As such, ECG measurements at the feet while standing are considered to be an unreliable trigger signal (low SNR). Therefore, it is often difficult to obtain a reliable cardiovascular trigger reference timing when using ECG sensors incorporated in base scale platform devices. Both Inan, et al. (IEEE Transactions on Information Technology in Biomedicine, 14:5, 1188-1196, 2010) and Shin, et al. (Physiological Measurement, 30, 679-693, 2009) have shown that the ECG component of the electrical signal measured between the two feet while standing was rapidly overpowered by the electromyogram (EMG) signal resulting from the leg muscle activity involved in maintaining balance.

The accuracy of cardiovascular information obtained from weighing scales is also influenced by measurement time. The number of beats obtained from heartbeats for signal averaging is a function of measurement time and heart rate. Typically, a resting heart rates range from 60 to 100 beats per minute. Therefore, short signal acquisition periods may yield a low number of beats to average, which may cause measurement uncertainty, also known as the standard error in the mean (SEM). SEM is the standard deviation of the sample mean estimate of a population mean. Where, SE is the standard error in the samples N, which is related to the standard error or the population S.

$$SE = \frac{S}{\sqrt{N}}$$

For example, a five second signal acquisition period may yield a maximum of five to eight beats for ensemble averaging, while a 10 second signal acquisition could yield 10-16 beats. However, the number of beats available for averaging and SNR determination is usually reduced for the following factors; (1) truncation of the first and last ensemble beat in the recording by the algorithm, (2) triggering beats falsely missed by triggering algorithm, (3) cardiorespiratory variability, (4) excessive body motion corrupting the trigger and Foot IPG signal, and (5) loss of foot contact with the measurement electrodes.

Sources of noise can require multiple solutions for SNR improvements for the signal being averaged. Longer measurement times increase the number of beats lost to truncation, false missed triggering, and excessive motion. Longer measurement times also reduce variability from cardiorespiratory effects. If shorter measurement times (e.g., less than 30 seconds) are desired for scale-based sensor platforms, sensing improvements need to tolerate body motion and loss of foot contact with the measurement electrodes.

The human cardiovascular system includes a heart with four chambers, separated by valves that return blood to the heart from the venous system into the right side of the heart, through the pulmonary circulation to oxygenate the blood, which then returns to the left side of the heart, where the oxygenated blood is pressurized by the left ventricles and is pumped into the arterial circulation, where blood is distributed to the organs and tissues to supply oxygen. The cardiovascular or circulatory system is designed to ensure oxygen availability and is often the limiting factor for cell survival. The heart normally pumps five to six liters of blood every minute during rest and maximum cardiac output during exercise increases up to seven-fold, by modulating heart rate and stroke volume. The factors that affect heart rate include autonomic innervation, fitness level, age and hormones. Factors affecting stroke volume include heart size, fitness level, contractility or pre-ejection period, ejection duration, preload or end-diastolic volume, afterload or systemic resistance. The cardiovascular system is constantly adapting to maintain a homeostasis (set point) that minimizes the work done by the heart to maintain cardiac output. As such, blood pressure is continually adjusting to minimize work demands during rest. Cardiovascular disease encompasses a variety of abnormalities in (or that affect) the cardiovascular system that degrade the efficiency of the system, which include but are not limited to chronically elevated blood pressure, elevated cholesterol levels, edema, endothelial dysfunction, arrhythmias, arterial stiffening, atherosclerosis, vascular wall thickening, stenosis, coronary artery disease, heart attack, stroke, renal dysfunction, enlarged heart, heart failure, diabetes, obesity and pulmonary disorders.

Each cardiac cycle results in a pulse of blood being delivered into the arterial tree. The heart completes cycles of atrial systole, delivering blood to the ventricles, followed by ventricular systole delivering blood into the lungs and the systemic arterial circulation, where the diastole cycle begins. In early diastole the ventricles relax and fill with blood, then in mid-diastole the atria and ventricles are relaxed and the ventricles continue to fill with blood. In late diastole, the sinoatrial node (the heart's pacemaker) depolarizes then contracting the atria, the ventricles are filled with more blood and the depolarization then reaches the atrioventricular node and enters the ventricular side beginning the systole phase. The ventricles contract and the blood is pumped from the ventricles to arteries.

The ECG is the measurement of the heart's electrical activity and is described in five phases. The P-wave represents atrial depolarization, the PR interval is the time between the P-wave and the start of the QRS complex. The QRS wave complex represents ventricular depolarization. The QRS complex is the strongest wave in the ECG and is frequently used as a timing reference for the cardiovascular cycle. Atrial repolarization is masked by the QRS complex. The ST interval represents the period of zero potential between ventricular depolarization and repolarization. The cycle concludes with the T-wave representing ventricular repolarization.

The blood ejected into the arteries creates vascular movements due to the blood's momentum. The blood mass ejected by the heart first travels headward in the ascending aorta and travels around the aortic arch then travels down the descending aorta. The diameter of the aorta increases during the systole phase due to the high compliance (low stiffness) of the aortic wall. Blood traveling in the descending aorta bifurcates in the iliac branch which transitions into a stiffer arterial region due to the muscular artery composition of the leg arteries. The blood pulsation continues down the leg and foot. Along the way, the arteries branch into arteries of smaller diameter until reaching the capillary beds where the pulsatile blood flow turns into steady blood flow, delivering oxygen to the tissues. The blood returns to the venous system terminating in the vena cava, where blood returns to the right atrium of the heart for the subsequent cardiac cycle.

Surprisingly, high quality simultaneous recordings of the Leg IPG and Foot IPG are attainable in a practical manner (e.g., a user operating the device correctly simply by standing on the impedance body scale foot electrodes), and can be used to obtain reliable trigger fiducial timings from the Leg IPG signal. This acquisition can be far less sensitive to motion-induced noise from the Leg EMG that often compromises Leg ECG measurements. Furthermore, it has been discovered that interleaving the two Kelvin electrode pairs for a single foot, result in a design that is insensitive to foot placement within the boundaries of the overall electrode area. As such, the user is not constrained to comply with accurate foot placement on conventional single foot Kelvin arrangements, which are highly prone to introducing motion artifacts into the IPG signal, or result in a loss of contact if the foot is slightly misaligned. Interleaved designs begin when one or more electrode surfaces cross over a single imaginary boundary line separating an excitation and sensing electrode pair. The interleaving is configured to maintain uniform foot surface contact area on the excitation and sensing electrode pair, regardless of the positioning of the foot over the combined area of the electrode pair.

Various aspects of the present disclosure include a weighing scale platform (e.g., scale 110) of an area sufficient for an adult of average size to stand comfortably still and minimize postural swaying. The nominal scale length (same orientation as foot length) is 12 inches and the width is 12 inches. The width can be increased to be consistent with the feet at shoulder width or slightly broader (e.g., 14 to 18 inches, respectively).

FIG. 1e is a flow chart depicting an example manner in which a user-specific physiologic meter or scale may be programmed in accordance with the present disclosure. This flow chart uses a computer processor circuit (or CPU) along with a memory circuit shown herein as user profile memory 146a. The CPU operates in a low-power consumption mode, which may be in off mode or a low-power sleep mode, and at least one other higher power consumption mode of operation. The CPU can be integrated with presence and/or motion sense circuits, such as a PIR circuit and/or pyro PIR circuit. In a typical application, the PIR circuit provides a constant flow of data indicative of amounts of radiation sensed in a field of view directed by the PIR circuit. For instance, the PIR circuit can be installed behind a transparent upper surface of the platform and installed at an angle so that the motion of the user approaching the platform apparatus is sensed. Radiation from the user, upon reaching a certain detectable level, wakes up the CPU which then transitions from the low-power mode, as depicted in block 140, to a regular mode of operation. Alternatively, the low-power mode of operation is transitioned from a response to another remote/wireless input used as a presence to awaken the CPU. In other embodiments, motion is sensed with a single integrated microphone or microphone array, to detect the sounds of a user approaching, or user motion can be detected by an accelerometer integrated in the scale.

Accordingly, from block 140, flow proceeds to block 142 where the user or other intrusion is sensed as data received at the platform apparatus. At block 144, the circuitry assesses whether the received data qualifies as requiring a wake up. If not, flow turns to block 140. If however, wake up is required, flow proceeds from block 144 to block 146 where the CPU assesses whether a possible previous user has approached the platform apparatus. This assessment is performed by the CPU accessing the user profile memory 146A and comparing data stored therein for one or more such previous users with criteria corresponding to the received data that caused the wake up. Such criteria includes, for example, the time of the day, the pace at which the user approached the platform apparatus as sensed by the motion detection circuitry, the height of the user as indicated by the motion sensing circuitry and/or a camera installed and integrated with the CPU, and/or more sophisticated bio-metric data provided by the user and/or automatically by the circuitry in the platform apparatus.

As discussed herein, such sophisticated circuitry can include one or more of the following user-specific attributes: foot length, type of foot arch, weight of user, and/or manner and speed at which the user steps onto the platform apparatus, or sounds made by the user's motion or by speech. As is also conventional, facial or body-feature recognition may also be used in connection with the camera and comparisons of images therefrom to images in the user profile memory.

From block 146, flow proceeds to block 148 where the CPU obtains and/or updates user corresponding data in the user profile memory. As a learning program is developed in the user profile memory, each access and use of the platform apparatus is used to expand on the data and profile for each such user. From block 148, flow proceeds to block 150 where a decision is made regarding whether the set of electrodes at the upper surface of the platform are ready for the user, such as may be based on the data obtained from the user profile memory. For example, delays may ensue from the user moving his or her feet about the upper surface of the platform apparatus, as may occur while certain data is being retrieved by the CPU (whether internally or from an external source such as a program or configuration data updates from the Internet cloud) or when the user has stepped over the user-display. If the electrodes are not ready for the user, flow proceeds from block 150 to block 152 to accommodate this delay.

Once the CPU determines that the electrodes are ready for use while the user is standing on the platform surface, flow proceeds to block 160. Stabilization of the user on the platform surface may be ascertained by injecting current through the electrodes via the interleaved arrangement thereof. Where such current is returned via other electrodes for a particular foot and/or foot size, and is consistent for a relatively brief period of time, for example, a few seconds, the CPU can assume that the user is standing still and ready to use the electrodes and related circuitry. At block 160, a decision is made that both the user and the platform apparatus are ready for measuring impedance and certain segments of the user's body, including at least one foot.

The remaining flow of FIG. 1e includes the application and sensing of current through the electrodes for finding the optimal electrodes (162) and for performing impedance measurements (block 164). These measurements are continued until completed at block 166 and all such useful measurements are recorded and are logged in the user profile memory for this specific user, at block 168. At block 172, the CPU generates output data to provide feedback as to the completion of the measurements and, as can be indicated as a request via the user profile for this user, as an overall report on the progress for the user and relative to previous measurements made for this user has stored in the user profile memory. Such feedback may be shown on the user-display, through a speaker with co-located apertures in the platform for audible reception by the user, and/or by vibration circuitry which, upon vibration under control of the CPU, the user can sense through one or both feet while standing on the scale. From this output at block 172, flow returns to the low power mode as indicated at block 174 with the return to the beginning of the flow at the block 140.

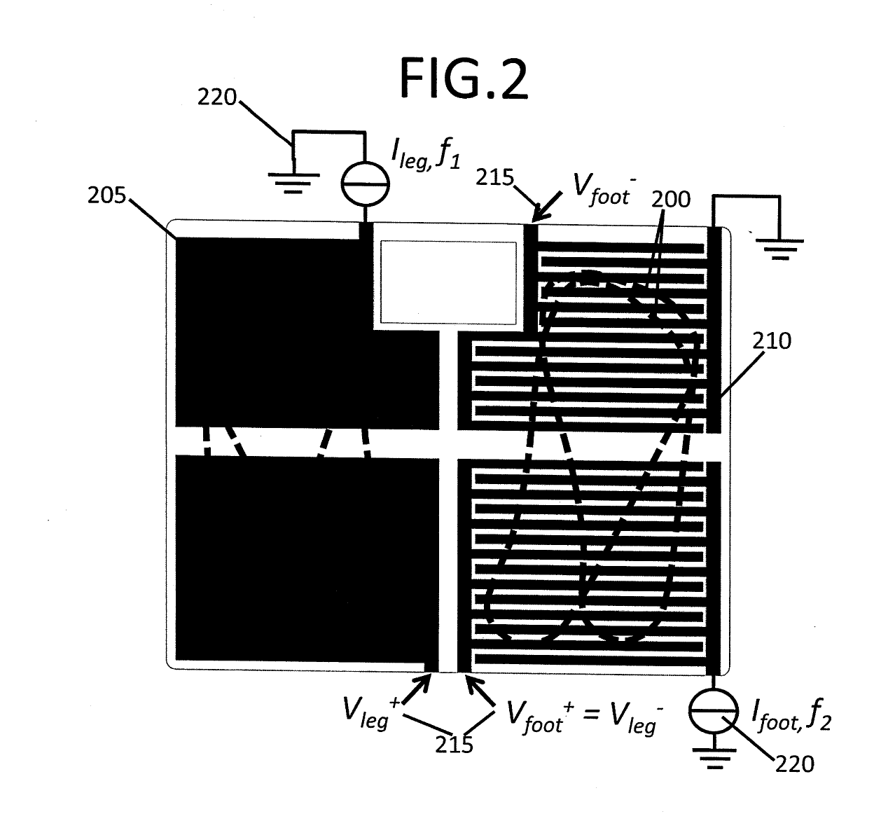

FIG. 2 shows an example of the insensitivity to foot placement 200 on scale electrode pairs 205/210 with multiple excitation paths 220 and sensing current paths 215, consistent with various aspects of the present disclosure. An aspect of the platform is that it has a thickness and strength to support a human adult of at least 200 pounds without fracturing, and another aspect of the device platform is comprised of at least six electrodes, where the first electrode pair 205 is solid and the second electrode pair 210 are interleaved. Another aspect is the first and second interleaved electrode pairs 205/210 are separated by a distance of at least 40+/−5 millimeters, where the nominal separation of less than 40 millimeters has been shown to degrade the single Foot IPG signal. Another key aspect is the electrode patterns are made from materials with low resistivity such as stainless steel, aluminum, hardened gold, ITO, index matched ITO (IMITO), carbon printed electrodes, conductive tapes, silver-impregnated carbon printed electrodes, conductive adhesives, and similar materials with resistivity lower than 300 ohms/sq. The resistivity can be below 150 ohms/sq. The electrodes are connected to the electronic circuitry in the scale by routing the electrodes around the edges of the scale to the surface below, or through at least one hole in the scale (e.g., a via hole).

Suitable electrode arrangements for dual Foot IPG measurements can be realized in other embodiments. In certain embodiments, the interleaved electrodes are patterned on the reverse side of a thin piece (e.g., less than 2 mm) of high-ion-exchange (HIE) glass, which is attached to a scale substrate and used in capacitive sensing mode. In certain embodiments, the interleaved electrodes are patterned onto a thin piece of paper or plastic which can be rolled up or folded for easy storage. In certain embodiments, the interleaved electrodes are integrated onto the surface of a tablet computer for portable IPG measurements. In certain embodiments, the interleaved electrodes are patterned onto a kapton substrate that is used as a flex circuit.

In certain embodiments, the scale area has a length of 10 inches with a width of eight inches for a miniature scale platform. Alternatively, the scale may be larger (up to 36 inches wide) for use in bariatric class scales.

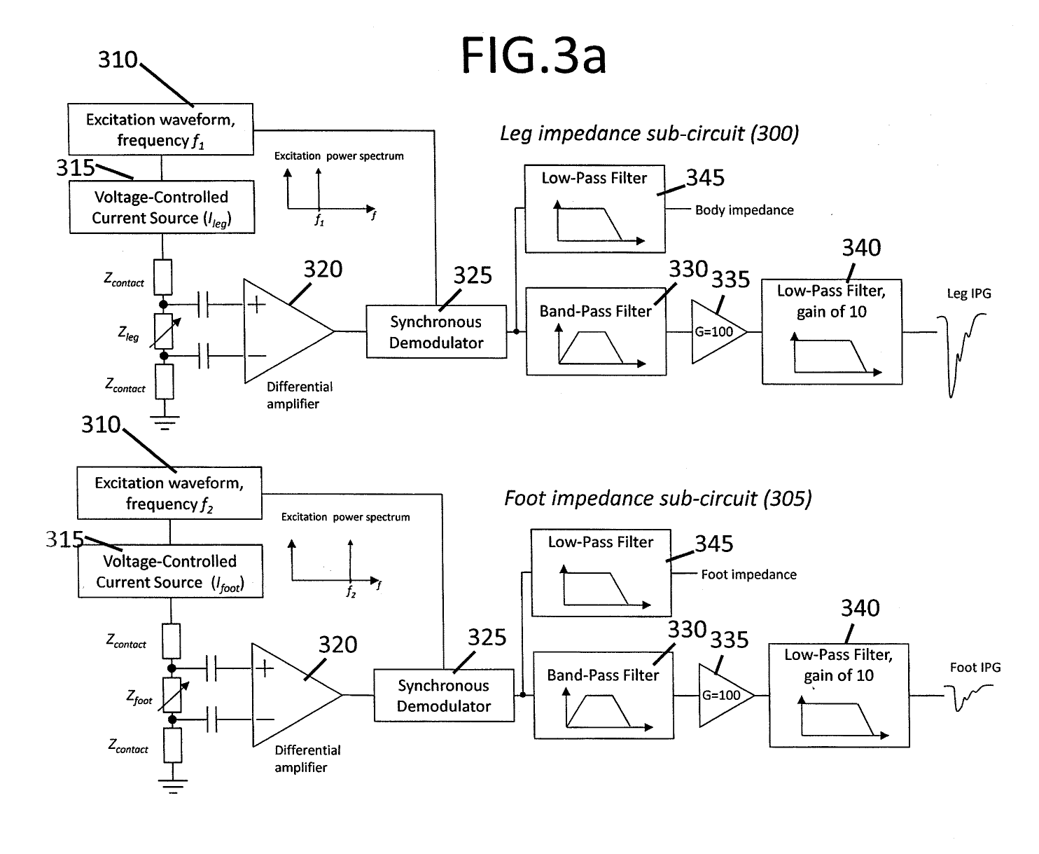

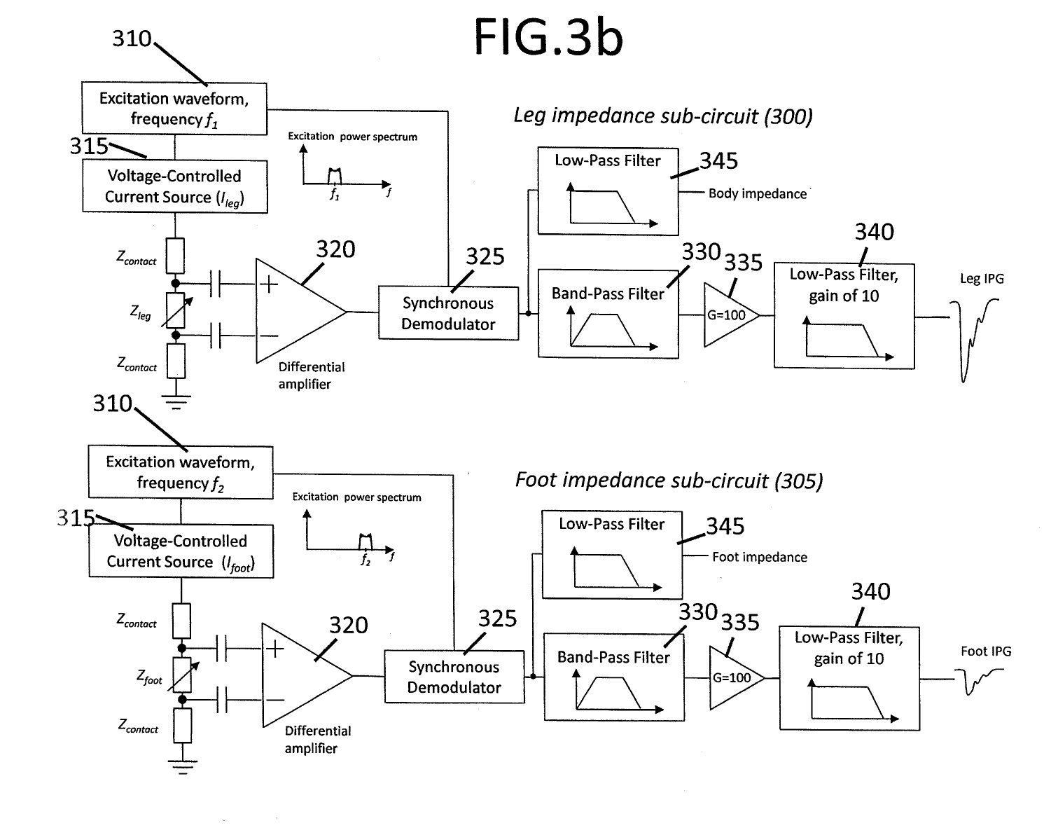

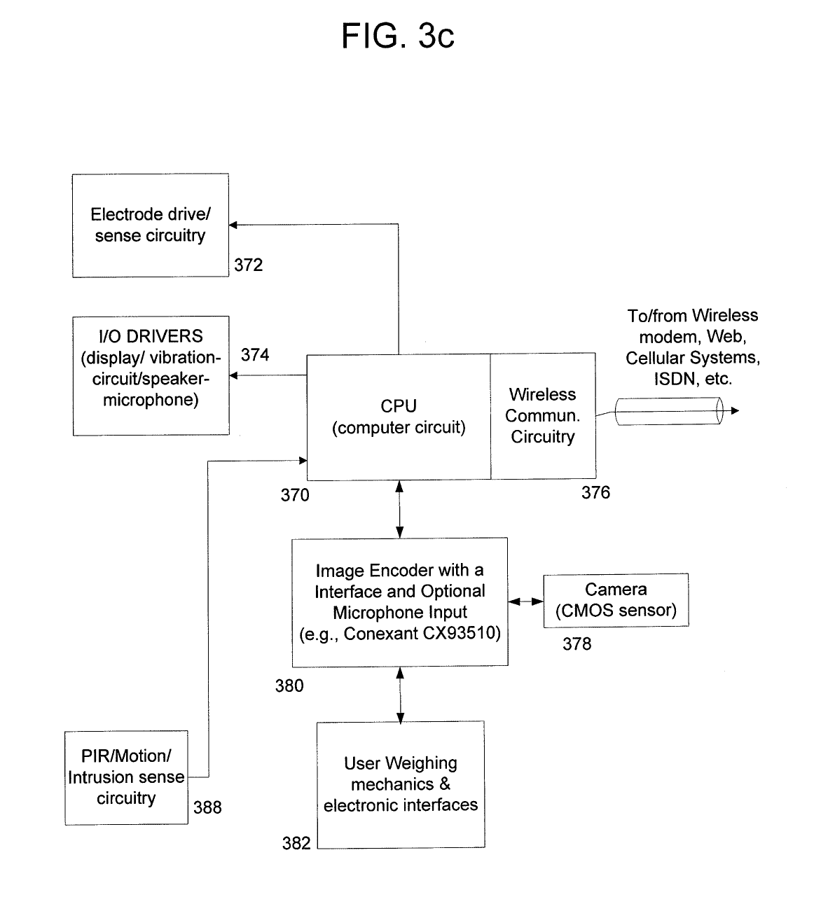

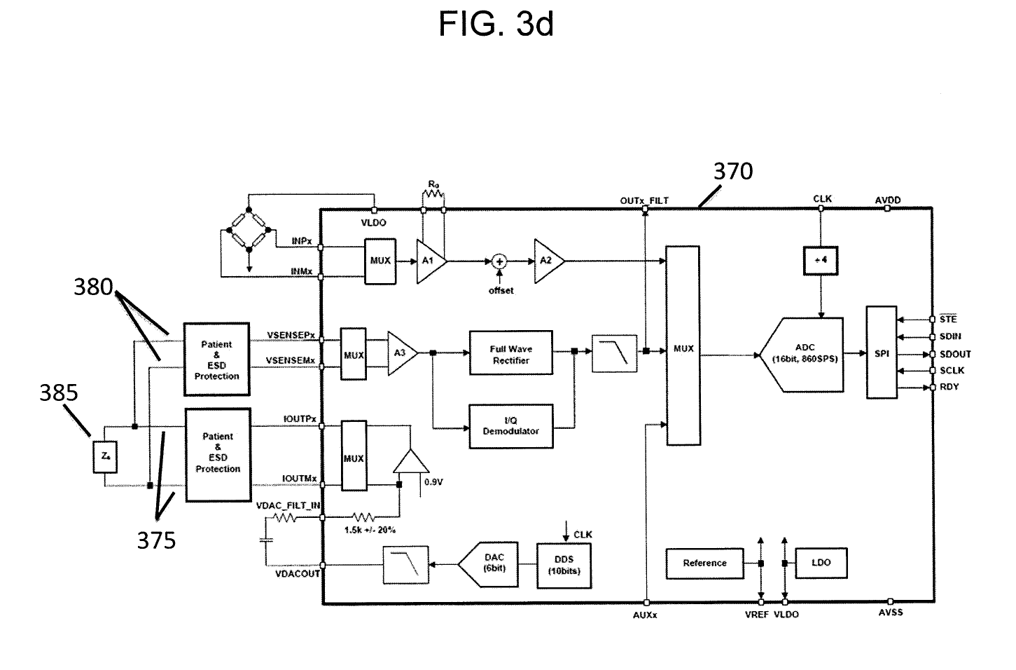

In the present disclosure, the leg and foot impedance measurements can be simultaneously carried out using a multi-frequency approach, in which the leg and foot impedances are excited by currents modulated at two different frequencies, and the resulting voltages are selectively measured using a synchronous demodulator as shown in FIG. 3a. This homodyning approach can be used to separate signals (in this case, the voltage drop due to the imposed current) with very high accuracy and selectivity.

This measurement configuration is based on a four-point configuration in order to minimize the impact of the contact resistance between the electrode and the foot, a practice well-known in the art of impedance measurement. In this configuration the current is injected from a set of two electrodes (the “injection” and “return” electrodes), and the voltage drop resulting from the passage of this current through the resistance is sensed by two separate electrodes (the “sense” electrodes), usually located in the path of the current. Since the sense electrodes are not carrying any current (by virtue of their connection to a high-impedance differential amplifier), the contact impedance does not significantly alter the sensed voltage.

In order to sense two distinct segments of the body (the legs and the foot), two separate current paths are defined by electrode positioning. Therefore two injection electrodes are used, each connected to a current source modulated at a different frequency. The injection electrode for leg impedance is located under the plantar region of the left foot, while the injection electrode for the Foot IPG is located under the heel of the right foot. Both current sources share the same return electrode located under the plantar region of the right foot. This is an illustrative example. Other configurations may be used.

The sensing electrodes can be localized so as to sense the corresponding segments. Leg IPG sensing electrodes are located under the heels of each foot, while the two foot sensing electrodes are located under the heel and plantar areas of the right foot. The inter-digitated nature of the right foot electrodes ensures a four-point contact for proper impedance measurement, irrespectively of the foot position, as already explained.

FIGS. 3a-3b show example block diagrams depicting the circuitry for sensing and measuring the cardiovascular time-varying IPG raw signals and steps to obtain a filtered IPG waveform, consistent with various aspects of the present disclosure. The example block diagrams shown in FIGS. 3a-3b are separated in to a leg impedance sub-circuit 300 and a foot impedance sub-circuit 305.

Excitation is provided by way of an excitation waveform circuit 310. The excitation waveform circuit 310 provides an excitation signal by way of a various types of frequency signals (as is shown in FIG. 3a) or, more specifically, a square wave signal (as shown in FIG. 3b). As is shown in FIG. 3b, the square wave signal is a 5 V at a frequency between 15,625 Hz and 1 MHz is generated from a quartz oscillator (such as an ECS-100AC from ECS International, Inc.) divided down by a chain of toggle flip-flops (e.g. a CD4024 from Texas Instruments, Inc.), each dividing stage providing a frequency half of its input (i.e., 1 Mhz, 500 kHz, 250 kHz, 125 kHz, 62.5 kHz, 31.250 kHz and 15.625 kHz). This (square) wave is then AC-coupled, scaled down to the desired amplitude and fed to a voltage-controlled current source circuit 315. The generated current is passed through a decoupling capacitor (for safety) to the excitation electrode, and returned to ground through the return electrode (grounded-load configuration). Amplitudes of 1 and 4 mA peak-to-peak are typically used for Leg and Foot IPGs, respectively.

The voltage drop across the segment of interest (legs or foot) is sensed using an instrumentation differential amplifier (e.g., Analog Devices AD8421) 320. The sense electrodes on the scale are AC-coupled to the input of the differential amplifier 320 (configured for unity gain), and any residual DC offset is removed with a DC restoration circuit (as exemplified in Burr-Brown App Note Application Bulletin, SBOA003, 1991, or Burr-Brown/Texas Instruments INA118 datasheet).

The signal is then demodulated with a synchronous demodulator circuit 325. The demodulation is achieved in this example by multiplying the signal by 1 or −1 synchronously with the current excitation. Such alternating gain is provided by an operational amplifier and an analog switch (SPST), such as an ADG442 from Analog Devices). More specifically, the signal is connected to both positive and negative inputs through 10 kOhm resistors. The output is connected to the negative input with a 10 kOhm resistor as well, and the switch is connected between the ground and the positive input. When open, the gain of the stage is unity. When closed (positive input grounded), the stage acts as an inverting amplifier of the gain −1. Alternatively, other demodulators such as analog multipliers or mixers can be used.

Once demodulated, the signal is band-pass filtered (0.480 Hz) with a first-order band-pass filter circuit 330 before being amplified with a gain of 100 with a non-inverting amplifier circuit 335 (e.g., using an LT1058 operational amplifier from Linear Technologies). The amplified signal is further amplified by 10 and low-pass filtered (cut-off at 30 Hz) using a low-pass filter circuit 340 such as 2-pole Sallen-Key filter stage with gain. The signal is then ready for digitization and further processing. In certain embodiments, the amplified signal can be passed through an additional low-pass filter circuit 345 to determine body or foot impedance.

In certain embodiments, the generation of the excitation voltage signal, of appropriate frequency and amplitude, is carried out by a microcontroller, such as MSP430 (Texas Instruments, Inc.). The voltage waveform can be generated using the on-chip timers and digital input/outputs or pulse width modulation (PWM) peripherals, and scaled down to the appropriate voltage through fixed resistive dividers, active attenuators/amplifiers using on-chip or off-chip operational amplifiers, as well as programmable gain amplifiers or programmable resistors. Alternatively, the waveforms can be directly generated by on- or off-chip digital-to-analog converters (DACs).

In certain embodiments, the shape of the excitation is not square, but sinusoidal. Such configuration would reduce the requirements on bandwidth and slew rate for the current source and instrumentation amplifier. Harmonics, potentially leading to higher electromagnetic interference (EMI), would also be reduced. Such excitation may also reduce electronics noise on the circuit itself. Lastly, the lack of harmonics from sine wave excitation may provide a more flexible selection of frequencies in a multi-frequency impedance system, as excitation waveforms have fewer opportunities to interfere between each other. Due to the concentration of energy in the fundamental frequency, sine wave excitation could also be more power-efficient. In certain embodiments, the shape of the excitation is not square, but trapezoidal.

To further reduce potential EMI, other strategies may be used, such as by dithering the square wave signal (i.e., introducing jitter in the edges following a fixed or random pattern) which leads to socalled spread spectrum signals, in which the energy is not localized at one specific frequency (or a set of harmonics), but rather distributed around a frequency (or a set of harmonics). An example of a spread-spectrum circuit suitable for Dual-IPG measurement is shown in FIG. 3b. Because of the synchronous demodulation scheme, phase-to-phase variability introduced by spread-spectrum techniques will not affect the impedance measurement. Such a spread-spectrum signal can be generated by, but not limited to, specialized circuits (e.g., Maxim MAX31C80, SiTime SiT9001), or generic microcontrollers (see Application Report SLAA291, Texas Instruments, Inc.). These spread-spectrum techniques can be combined with clock dividers to generate lower frequencies as well.

As may be clear to one skilled in the art, these methods of simultaneous measurement of impedance in the leg and foot can be used for standard Body Impedance Analysis (BIA), aiming at extracting relative content of total water, free-water, fat mass and others. Impedance measurements for BIA are typically done at frequencies ranging from kilohertz up to several megahertz. The multi-frequency measurement methods described above can readily be used for such BIA, provided the circuit can be modified so that the DC component of the impedance is not canceled by the instrumentation amplifier (no DC restoration circuit used). The high-pass filter can be implemented after the instrumentation amplifier, enabling the measurement of the DC component used for BIA. This multi-frequency technique can be combined with traditional sequential measurements used for BIA, in which the impedance is measured at several frequencies sequentially. These measurements are repeated in several body segments for segmental BIAs, using a switch matrix to drive the current into the desired body segments.

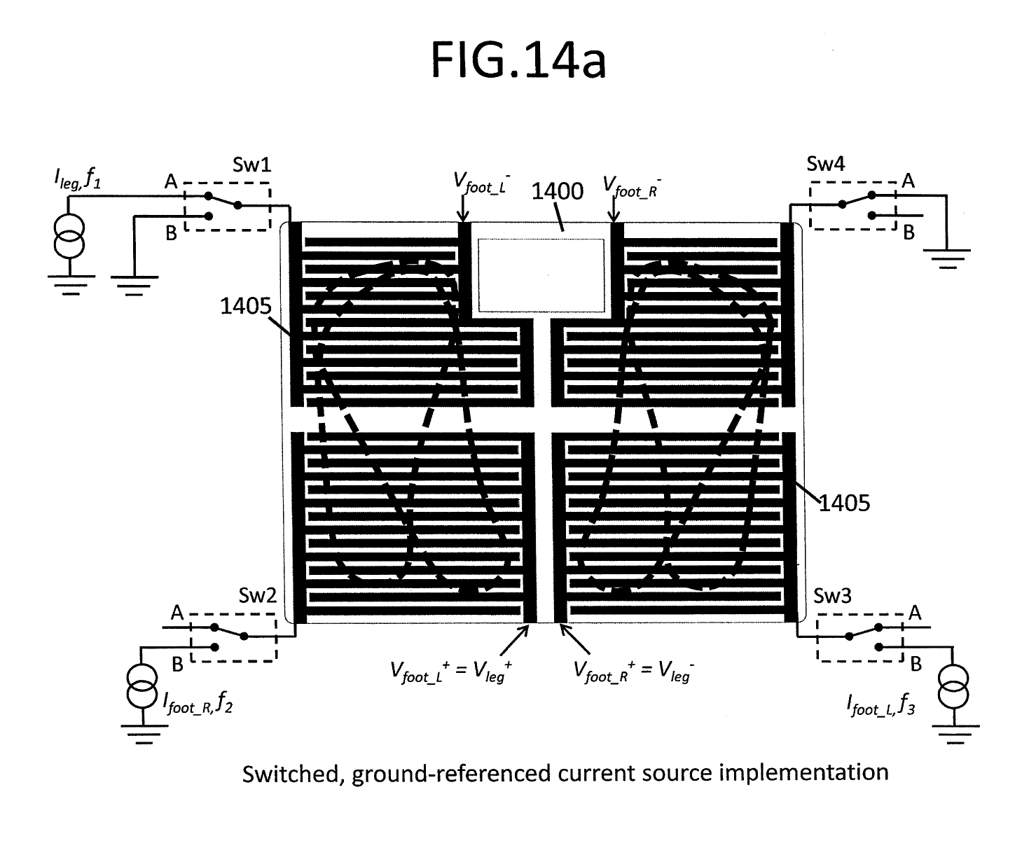

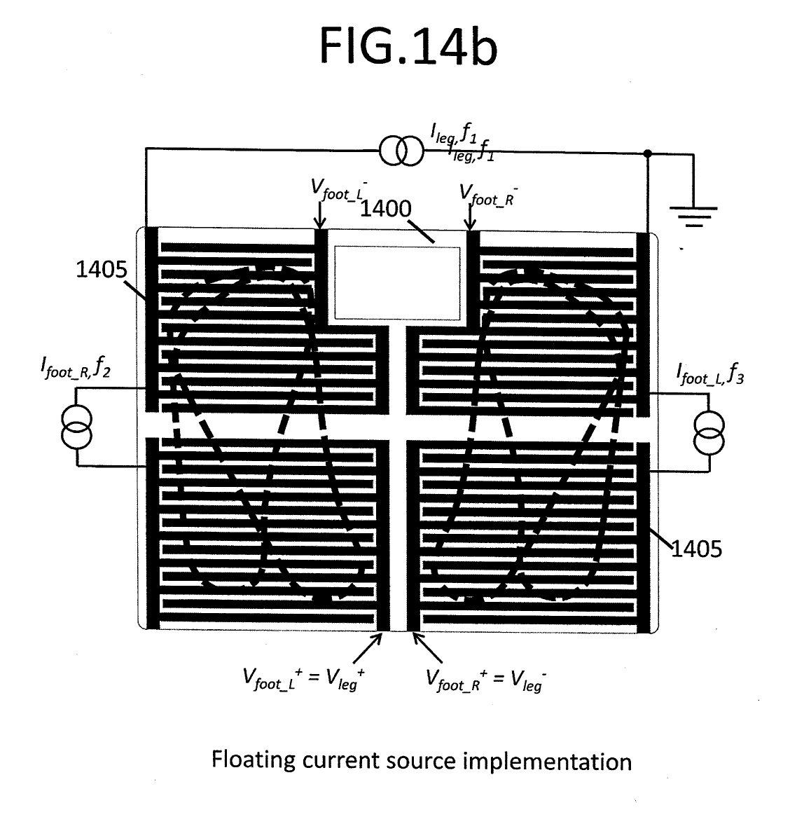

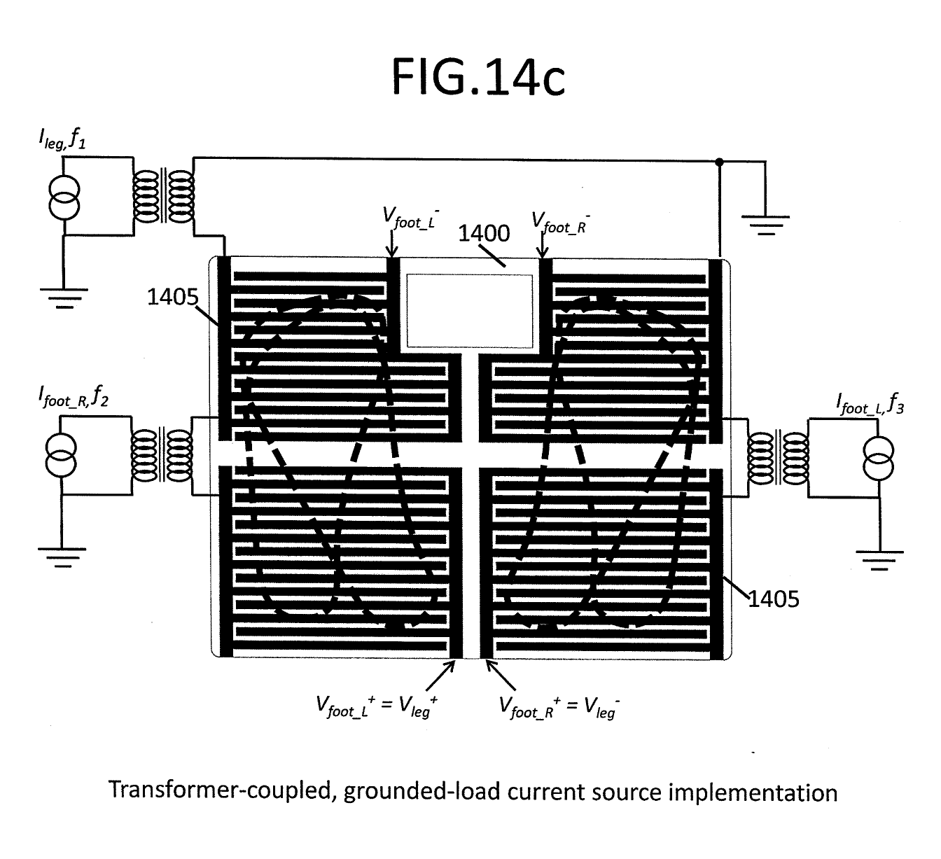

While FIG. 2 shows a circuit and electrode configuration suitable to measure two different segments (legs and one foot), this approach is not readily extendable to more segments due to the shared current return electrode (ground). To overcome this limitation, and provide simultaneous measurements in both feet, the system can be augmented with analog switches to provide time-multiplexing of the impedance measurements in the different segments. This multiplexing can be a one-time sequencing (each segment is measured once), or interleaved at a high-enough frequency that the signal can be simultaneously measured on each segment. The minimum multiplexing rate for proper reconstruction is twice the bandwidth of the measured signal, based on signal processing theory, which equals to about 100 Hz for the impedance signal considered here. The rate must also allow for the signal path to settle in between switching, usually limiting the maximum multiplexing rate. Referring to FIG. 14a, one cycle might start the measurement of the leg impedance and left foot impedances (similarly to previously described, sharing a common return electrode), but then follow with a measurement of the right foot after reconfiguring the switches. For specific information regarding typical switch configurations, reference to U.S. patent application Ser. No. 14/338,266 filed on Oct. 7, 2015, which is fully incorporated for its specific and general teaching of switch configurations.

Since right and left feet are measured sequentially, one should note that a unique current source (at the same frequency) may be used to measure both, providing that the current source is not connected to the two feet simultaneously through the switches, in which case the current would be divided between two paths. One should also note that a fully-sequential measurement, using a single current source (at a single frequency) successively connected to the three different injection electrodes, could be used as well, with the proper switch configuration sequence (no split current path).

In certain embodiments, the measurement of various body segments, and in particular the legs, right foot and left foot, is achieved simultaneously due to as many floating current sources as segments to be measured, running at separate frequency so they can individually be demodulated. Such configuration is exemplified in FIG. 14b for three segments (legs, right and left feet). Such configuration has the advantage to provide true simultaneous measurements without the added complexity of time-multiplexing/demultiplexing, and associated switching circuitry. An example of such floating current source is found in Plickett, et al., Physiological Measurement, 32 (2011). Another approach to floating current sources is the use of transformer-coupled current sources (as depicted in FIG. 14c). Using transformers to inject current into the electrodes enables the use of simpler, grounded-load current sources on the primary, while the electrodes are connected to the secondary. Turn ratio would typically be 1:1, and since frequencies of interest for impedance measurement are typically in the 10-1000 kHz (occasionally 1 kHz for BIA), relatively small transformers can be used. In order to limit the common mode voltage of the body, one of the electrodes in contact with the foot can be grounded.

While certain embodiments presented in the above specification have used current sources for excitation, the excitation can also be performed by a voltage source, where the resulting injection current is monitored by a current sense circuit so that impedance can still be derived by the ratio of the sensed voltage (on the sense electrodes) over the sensed current (injected in the excitation electrodes). It should be noted that broadband spectroscopy methods could also be used for measuring impedances at several frequencies. Combined with time-multiplexing and current switching described above, multi-segment broadband spectroscopy can be achieved.

Various aspects of the present disclosure are directed toward robust timing extraction of the blood pressure pulse in the foot which is achieved by means of a two-step processing. In a first step, the usually high-SNR Leg IPG is used to derive a reference (trigger) timing for each heart pulse. In a second step, a specific timing in the lower-SNR Foot IPG is extracted by detecting its associated feature within a restricted window of time around the timing of the Leg IPG.

Consistent with yet further embodiments of the present disclosure, FIG. 3c depicts an example block diagram of circuitry for operating core circuits and modules, including, for example, the operation of the CPU as in FIG. 1a with the related more specific circuit blocks/modules in FIGS. 3A-3B. As shown in the center of FIG. 3c, the computer circuit 370 is shown with other previously-mentioned circuitry in a generalized manner without showing some of the detailed circuitry (e.g., amplification and current injection/sensing (372)). The computer circuit 370 can be used as a control circuit with an internal memory circuit (or as integrated with the memory circuit for the user profile memory 146A of FIG. 1a) for causing, processing and/or receiving sensed input signals as at block 372. As discussed, these sensed signals can be responsive to injection current and/or these signals can be sensed by less complex grid-based sense circuitry surrounding the platform as is convention in capacitive touch-screen surfaces which, in certain embodiments, the platform includes.

As noted, the memory circuit can be used not only for the user profile memory, but also as to provide configuration and/or program code and/or other data such as user-specific data from another authorized source such as from a user monitoring his/her logged data and/or profile from a remote desk-top. The remote device or desk-top can communicate with and access such data via a wireless communication circuit 376. For example, the wireless communication circuit 376 provides an interface between an app on the user's cellular telephone/tablet and the apparatus, wherefrom the IPhone is the output/input interface for the platform (scale) apparatus including, for example, an output display, speaker and/or microphone, and vibration circuitry; each of these I/O aspects and components being discussed herein in connection with other example embodiments.

A camera 378 and image encoder circuit 380 (with compression and related features) can also be incorporated as an option. As discussed above, the weighing scale components, as in block 382, are also optionally included in the housing which encloses and/or surrounds the platform.

For long-lasting battery life in the platform apparatus (batteries not shown), at least the CPU 370, the wireless communication circuit 376, and other current draining circuits are inactive unless and until activated in response to the intrusion/sense circuitry 388. As shown, one specific implementation employs a Conexant chip (e.g., CX93510) to assist in the low-power operation. This type of circuitry is designed for motion sensors configured with a camera for visual verification and image and video monitoring applications (such as by supporting JPEG and MJPEG image compression and processing for both color and black and white images). When combined with an external CMOS sensor, the chip retrieves and stores compressed JPEG and audio data in an on-chip memory circuit (e.g., 256 KB/128 KB frame buffer) to alleviate the necessity of external memory. The chip uses a simple register set via the microprocessor interface and allows for wide flexibility in terms of compatible operation with another microprocessor.

In one specific embodiment, a method of using the platform with the plurality of electrodes are concurrently contacting a limb of the user, includes operating such to automatically obtain measurement signals from the plurality of electrodes. As noted above, these measurement signals might initially be through less complex (e.g., capacitive grid-type) sense circuitry. Before or while obtaining a plurality of measurement signals by operating the circuitry, the signal-sense circuitry 388 is used to sense wireless-signals indicative of the user approaching the platform and, in response, causing the CPU circuitry 370 to transition from a reduced power-consumption mode of operation and at least one higher power-consumption mode of operation. After the circuitry is operating in the higher power-consumption mode of operation, the CPU accesses the user-corresponding data stored in the memory circuit and causes a plurality of impedance-measurement signals to be obtained by using the plurality of electrodes while they are contacting the user via the platform; therefrom, the CPU generates signals corresponding to cardiovascular timings of the user.

The signal-sense circuit can be employed as a passive infrared detector and with the CPU programmed (as a separate module) to evaluate whether radiation from the passive infrared detector is indicative of a human. For example, sensed levels of radiation that would correspond to a live being that has a size which is less than a person of a three-foot height, and/or not being sensed as moving for more than a couple seconds, can be assessed as being a non-human.

Accordingly, as the user is recognized as being human, the CPU is activated and begins to attempt the discernment process of which user might be approaching. This is performed by the CPU accessing the user-corresponding data stored in the memory circuit (the user profile memory). If the user is recognized based on parameters such as discussed above (e.g., time of morning, speed of approach, etc.), the CPU can also select one of a plurality of different types of user-discernible visual/audible/tactile information and for presenting the discerned user with visual/audible/tactile information that was retrieved from the memory as being specific to the user. For example, user-selected visual/audible data can be outputted for the user. Also, responsive to the motion detection indication, the camera can be activated to capture at least one image of the user while the user is approaching the platform (and/or while the user is on the platform to log confirmation of the same user with the measured impedance information). As shown in block 374 of FIG. 3c, where a speaker is also integrated with the CPU, the user can simply command the platform apparatus to start the process and activation proceeds.

In another method, the circuitry of FIG. 3c is used with the electrodes being interleaved and engaging the user, as a combination weighing scale (via block 382) and a physiologic user-specific impedance-measurement device. By using the impedance-measurement signals and obtaining at least two impedance-measurement signals between one foot of the user and another location of the user, the interleaved electrodes assist the CPU in providing measurement results that indicate one or more of the following user-specific attributes as being indicative or common to the user: foot impedance, foot length, and type of arch, and wherein one or more of the user-specific attributes are accessed in the memory circuit and identified as being specific to the user. This information can be later retrieved by the user, medical and/or security personnel, according to a data-access authorization protocol as might be established upon initial configuration for the user.

FIG. 3d shows an exemplary block diagram depicting the circuitry for interpreting signals received from electrodes (e.g., 372 of FIG. 3c), and/or CPU 370 of FIG. 3c. The input electrodes 375 transmit electrical signals through the patient's body (depending on the desired biometric and physiological test to be conducted) and output electrodes 380 receive the modified signal as affected by a user's electrical impedance 385. Once received by the output electrodes 380, the modified signal is processed by processor circuitry 370 based on the selected test. Signal processing conducted by the processor circuitry 370 is discussed in more detail above (with regard to FIGS. 3a-b). In certain embodiments of the present disclosure, the circuitry within 370 is provided by Texas Instruments part # AFE4300.

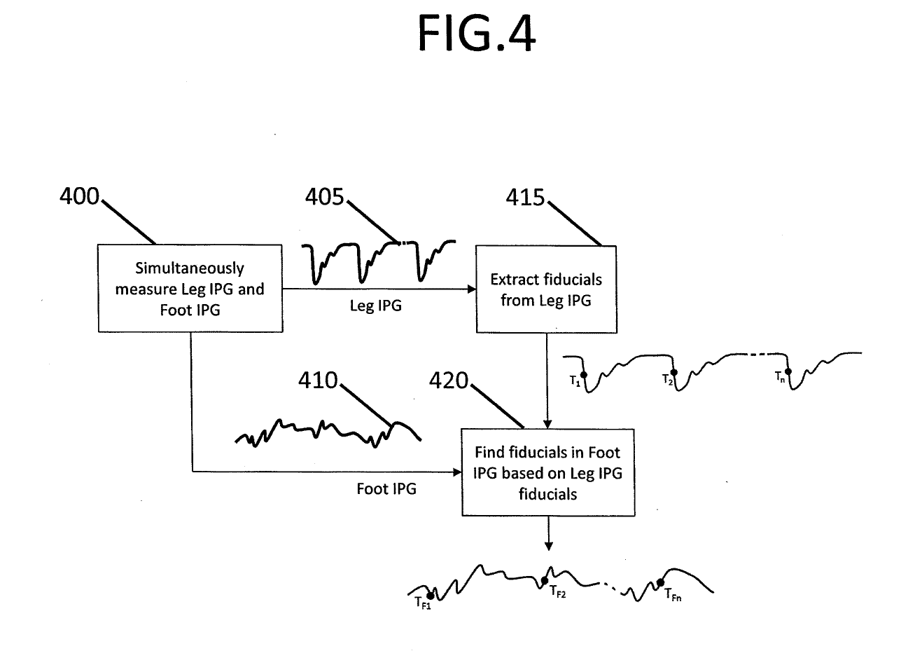

FIG. 4 shows an example block diagram depicting signal processing steps to obtain fiducial references from the individual Leg IPG “beats,” which are subsequently used to obtain fiducials in the Foot IPG, consistent with various aspects of the present disclosure. In the first step, as shown in block 400, the Leg IP and the Foot IPG are simultaneously measured. As shown at 405, the Leg IPG is low-pass filtered at 20 Hz with an 8-pole Butterworth filter, and inverted so that pulses have an upward peak. The location of the pulses is then determined by taking the derivative of this signal, integrating over a 100 ms moving window, zeroing the negative values, removing the large artifacts by zeroing values beyond 15× the median of the signal, zeroing the values below a threshold defined by the mean of the signal, and then searching for local maxima. Local maxima closer than a defined refractory period of 300 ms to the preceding ones are dismissed. The result is a time series of pulse reference timings.

As is shown in 410, the foot IPG is low-pass filtered at 25 Hz with an 8-pole Butterworth filter and inverted (so that pulses have an upward peak). Segments starting from the timings extracted (415) from the Leg IPG (reference timings) and extending to 80% of the previous pulse interval, but no longer than one second, are defined in the Foot IPG. This defines the time windows where the Foot IPG is expected to occur, avoiding misdetection outside of these windows. In each segment, the derivative of the signal is computed, and the point of maximum positive derivative (maximum acceleration) is extracted. The foot of the IPG signal is then computed using an intersecting tangent method, where the fiducial (420) is defined by the intersection between a first tangent to the IPG at the point of maximum positive derivative and a second tangent to the minimum of the IPG on the left of the maximum positive derivative within the segment.

The time series resulting from this two-step extraction is used with another signal to facilitate further processing. These timings are used as reference timings to improve the SNR of BCG signals to extract intervals between a timing of the BCG (typically the I-wave) and the Foot IPG for the purpose of computing the PWV, as previously disclosed in U.S. 2013/0310700 (Wiard). In certain embodiments, the timings of the Leg IPG are used as reference timings to improve the SNR of BCG signals, and the foot IPG timings are used to extract intervals between timing fiducials of the improved BCG (typically the I-wave) and the Foot IPG for the purpose of computing the PTT and the (PWV).

In certain embodiments, the processing steps include an individual pulse SNR computation after individual timings are extracted, either in Leg IPG or Foot IPG. Following the computation of the SNRs, pulses with a SNR below a threshold value are eliminated from the time series, to prevent propagating noise. The individual SNRs may be computed in a variety of methods known to one skilled in the art. For instance, an estimated pulse can be computed by ensemble averaging segments of signal around the pulse reference timing. The noise associated with each pulse is defined as the difference between the pulse and the estimated pulse. The SNR is the ratio of the root-mean-square (RMS) value of the estimated pulse over the RMS value of the noise for that pulse.

In certain embodiments, the time interval between the Leg IPG pulses, and the Foot IPG pulses, also detected by the above-mentioned methods, is extracted. The Leg IPG measuring a pulse occurring earlier in the legs compared to the pulse from the Foot IPG, the interval between these two is related to the propagation speed in the lower body, i.e., the peripheral vasculature. This provides complementary information to the interval extracted between the BCG and the Foot IPG for instance, and is used to decouple central versus peripheral vascular properties. It is also complementary to information derived from timings between the BCG and the Leg ICG.

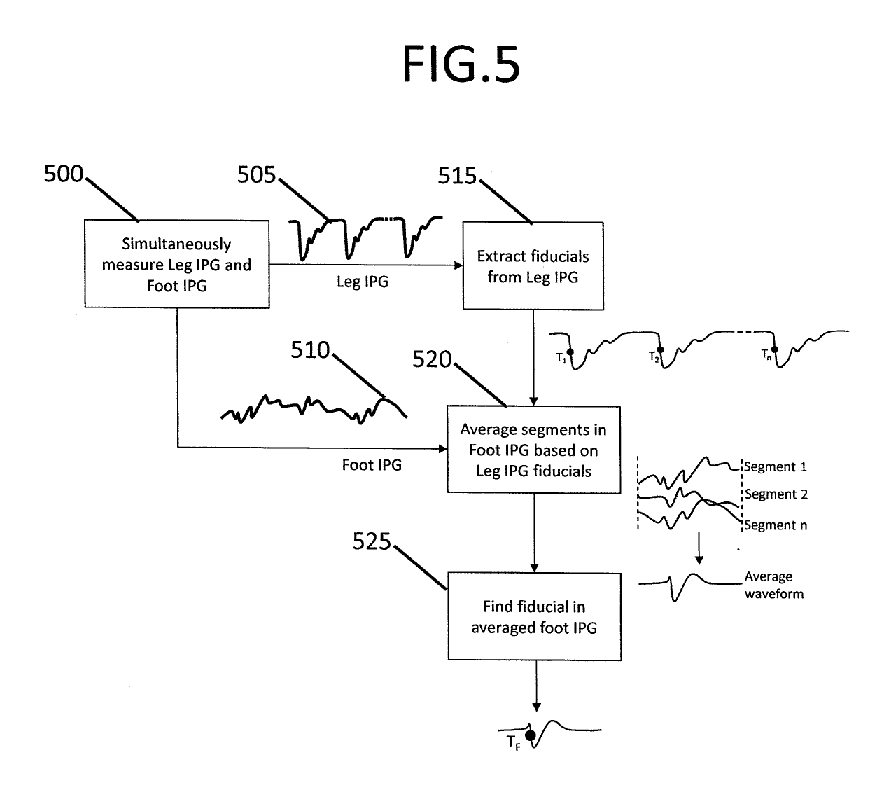

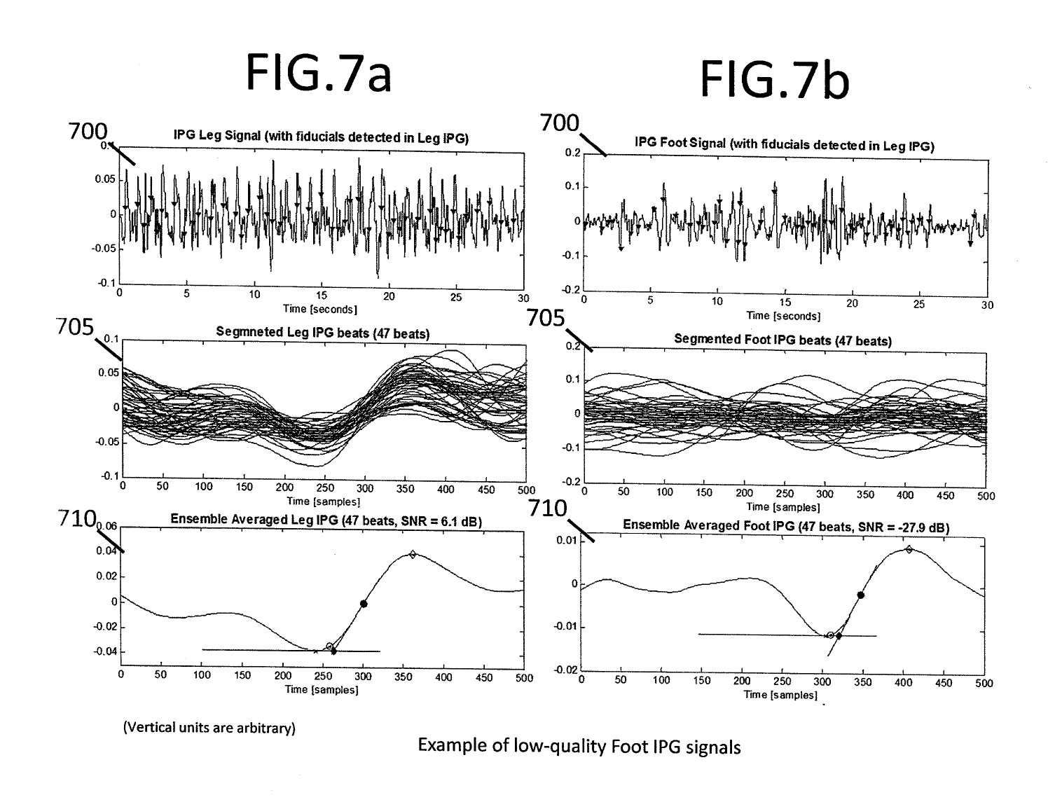

FIG. 5 shows an example flowchart depicting signal processing to segment individual Foot IPG “beats” to produce an averaged IPG waveform of improved SNR, which is subsequently used to determine the fiducial of the averaged Foot IPG, consistent with various aspects of the present disclosure. Similar to the method shown in FIG. 4, the Leg IP and the Foot IPG are simultaneously measured (500), the Leg IPG is low-pass filtered (505), the foot IPG is low-pass filtered (510), and segments starting from the timings extracted (515) from the Leg IPG (reference timings). The segments of the Foot IPG extracted based on the Leg IPG timings are ensemble-averaged (520) to produce a higher SNR Foot IPG pulse. From this ensemble-averaged signal, the start of the pulse is extracted using the same intersecting tangent approach as described earlier. This approach enables the extraction of accurate timings in the Foot IPG even if the impedance signal is dominated by noise, as shown in FIG. 7b. These timings are used together with timings extracted from the BCG for the purpose of computing the PTT and (PWV). Timings derived from ensemble-averaged waveforms and individual waveforms can also be both extracted, for the purpose of comparison, averaging and error-detection.

Specific timings extracted from the IPG pulses (from either leg or foot) are related (but not limited) to the peak of the pulse, the minimum preceding the peak, or the maximum second derivative (maximum rate of acceleration) preceding the point of maximum derivative. An IPG pulse and the extraction of a fiducial (525) in the IPG can be performed by other signal processing methods, including (but not limited to) template matching, cross-correlation, wavelet-decomposition, or short window Fourier transform.

FIG. 6a shows examples of the Leg IPG signal with fiducials (plot 600); the segmented Leg IPG into beats (plot 605); and the ensemble-averaged Leg IPG beat with fiducials and calculated SNR (plot 610), for an exemplary high-quality recording, consistent with various aspects of the present disclosure.

FIG. 6b shows examples of the Foot IPG signal with fiducials derived from the Leg IPG fiducials (plot 600); the segmented Foot IPG into beats (plot 605); and the ensemble-averaged Foot IPG beat with fiducials and calculated SNR (plot 610), for an exemplary high-quality recording, consistent with various aspects of the present disclosure.

FIG. 7a shows examples of the Leg IPG signal with fiducials (plot 700); the segmented Leg IPG into beats (plot 705); and the ensemble averaged Leg IPG beat with fiducials and calculated SNR (plot 710), for an exemplary low-quality recording, consistent with various aspects of the present disclosure.

FIG. 7b shows examples of the Foot IPG signal with fiducials derived from the Leg IPG fiducials (plot 700); the segmented Foot IPG into beats (plot 705); and the ensemble-averaged Foot IPG beat with fiducials and calculated SNR (plot 710), for an exemplary low-quality recording, consistent with aspects of the present disclosure.

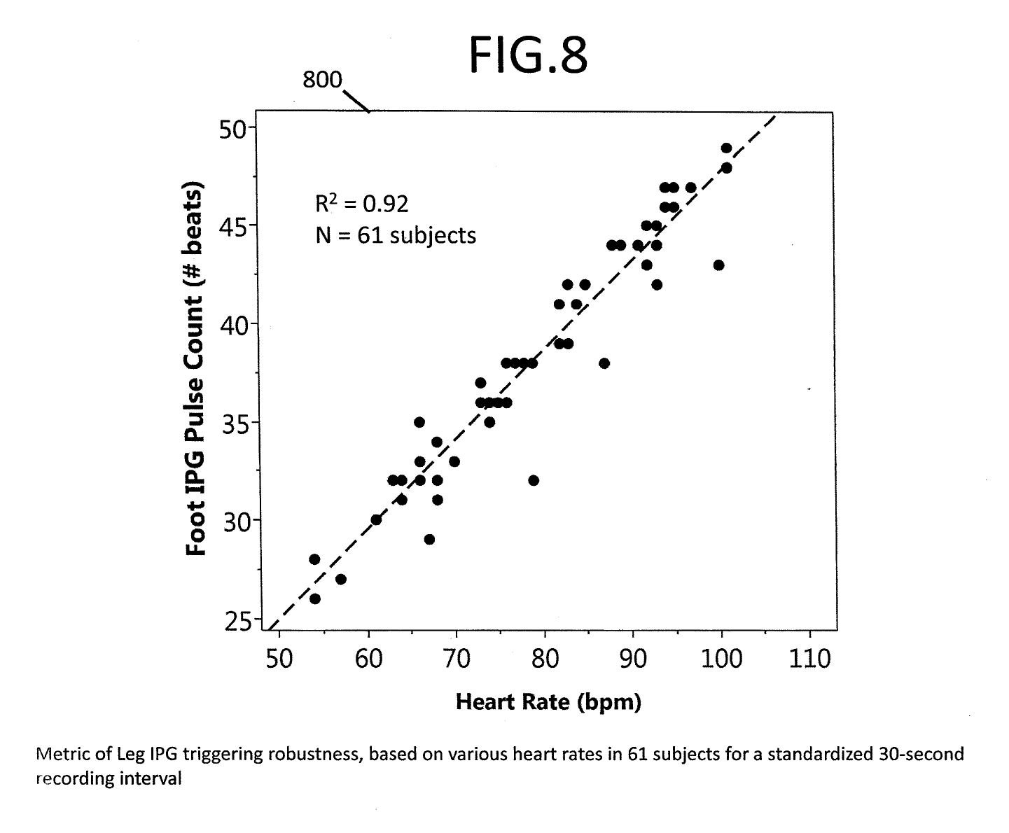

FIG. 8 shows an example correlation plot 800 for the reliability in obtaining the low SNR Foot IPG pulse for a 30-second recording, using the first impedance signal as the trigger pulse, from a study including 61 test subjects with various heart rates, consistent with various aspects of the present disclosure.

In certain embodiments, a dual-Foot IPG is measured, allowing the detection of blood pressure pulses in both feet. Such information can be used for diagnostic of peripheral arterial diseases (PAD) by comparing the relative PATs in both feet to look for asymmetries. It can also increase the robustness of the measurement by allowing one foot to have poor contact with electrodes (or no contact at all). SNR measurements can be used to assess the quality of the signal in each foot, and to select the best one for downstream analysis. Timings extracted from each foot can be compared and set to flag potentially inaccurate PWV measurements due to arterial peripheral disease, in the event these timings are different by more than a threshold. Alternatively, timings from both feet are pooled to increase the overall SNR if their difference is below the threshold.

In certain embodiments, the disclosure is used to measure a PWV, where the IPG is augmented by the addition of BCG sensing into the weighing scale to determine characteristic fiducials between the BCG and Leg IPG trigger, or the BCG and Foot IPG. The BCG sensors are comprised typically of the same strain gage set used to determine the bodyweight of the user. The load cells are typically wired into a bridge configuration to create a sensitive resistance change with small displacements due to the ejection of the blood into the aorta, where the circulatory or cardiovascular force produce movements within the body on the nominal order of 1-3 Newtons. BCG forces can be greater than or less than the nominal range in cases such as high or low cardiac output.

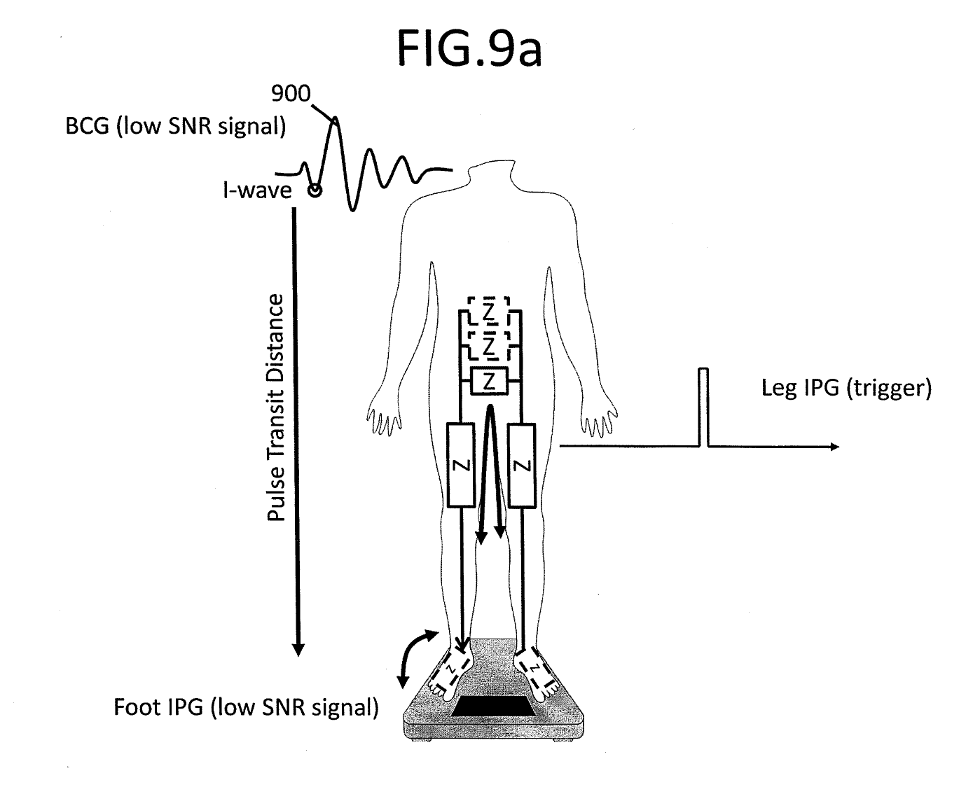

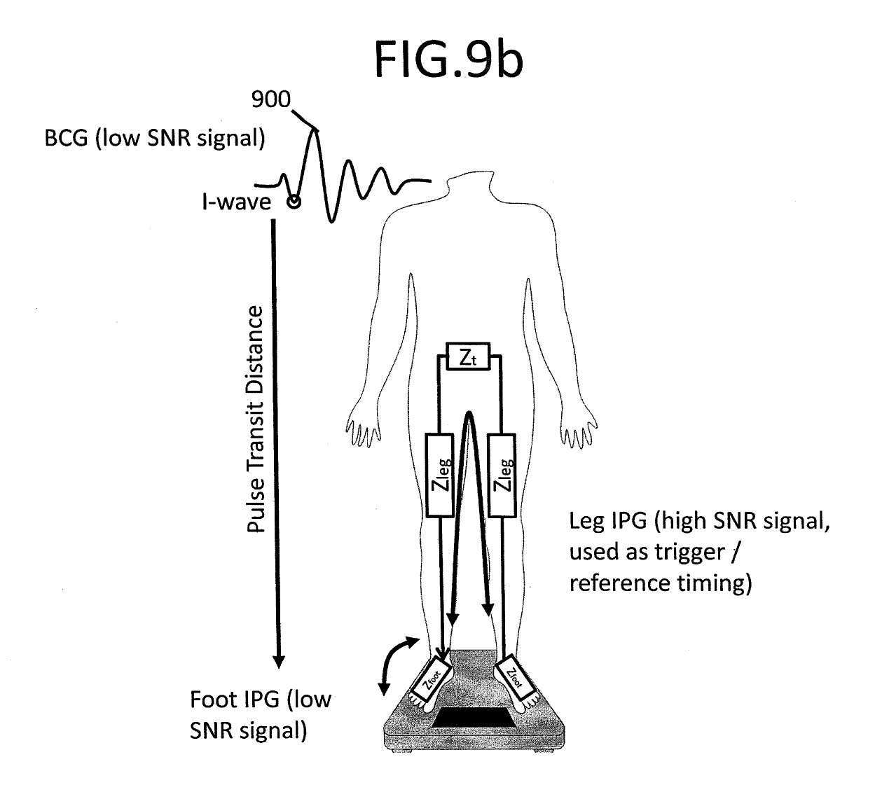

FIGS. 9a-b show example configurations to obtain the PTT, using the first IPG as the triggering pulse for the Foot IPG and BCG, consistent with various aspects of the present disclosure. The I-wave of the BCG 900 normally depicts the headward force due to cardiac ejection of blood into the ascending aorta which is used as a timing fiducial indicative of the pressure pulse initiation of the user's proximal aorta relative to the user's heart. The J-wave is indicative of timings in the systole phase and also incorporates information related to the strength of cardiac ejection and the ejection duration. The K-Wave provides systolic and vascular information of the user's aorta. The characteristic timings of these and other BCG waves are used as fiducials that can be related to fiducials of the IPG signals of the present disclosure.

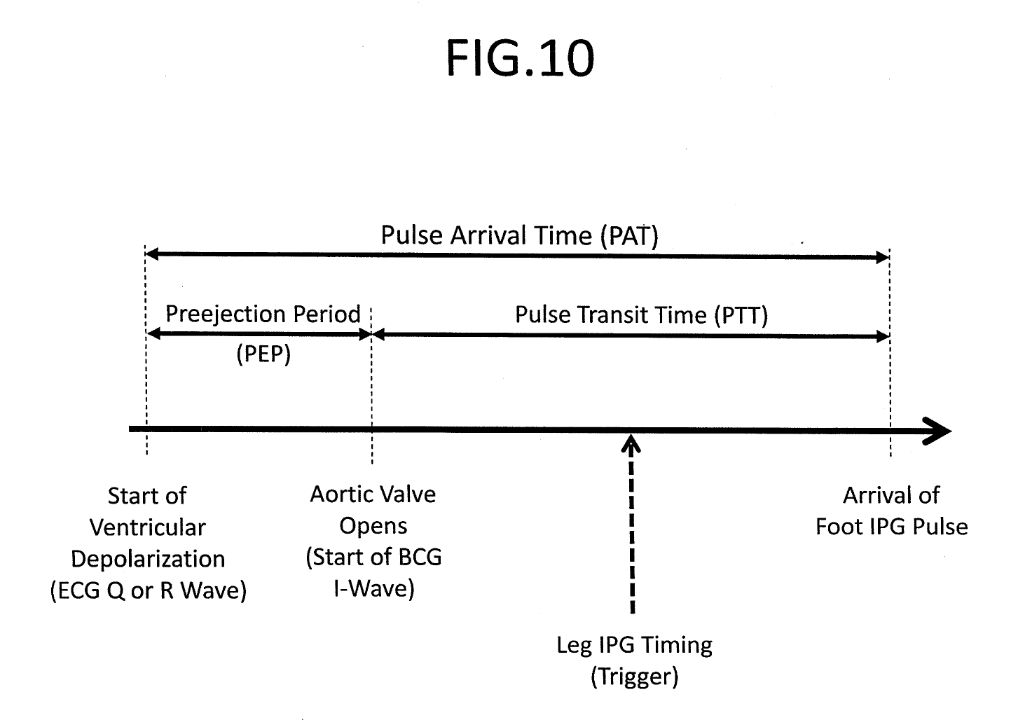

FIG. 10 shows nomenclature and relationships of various cardiovascular timings, consistent with various aspects of the present disclosure.

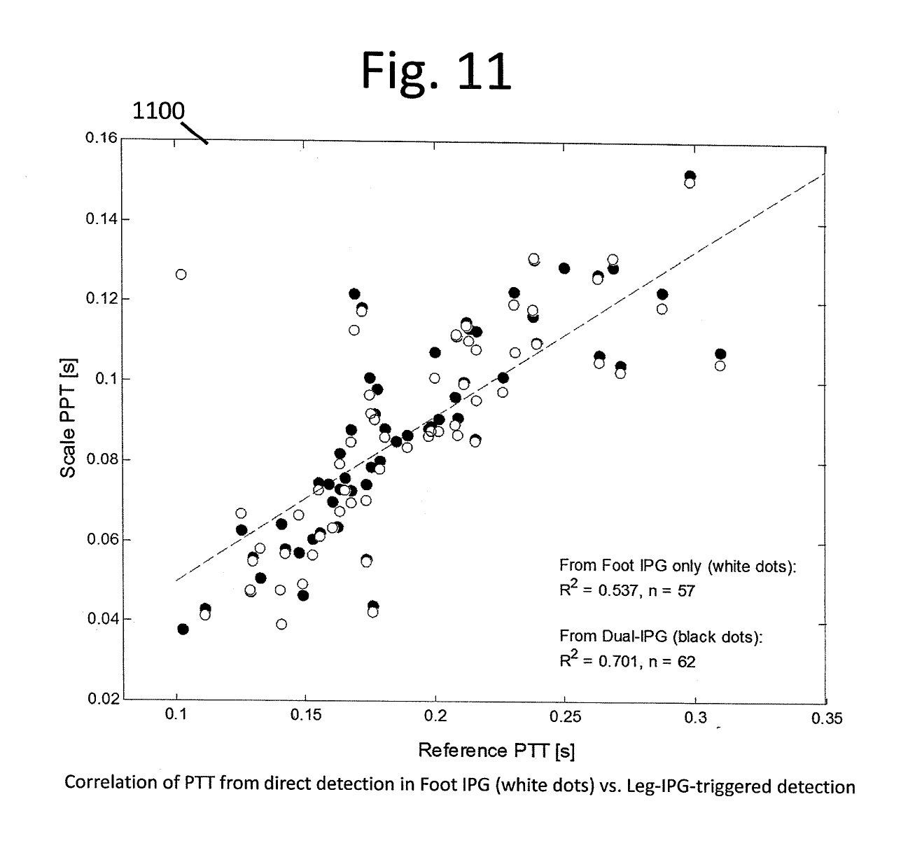

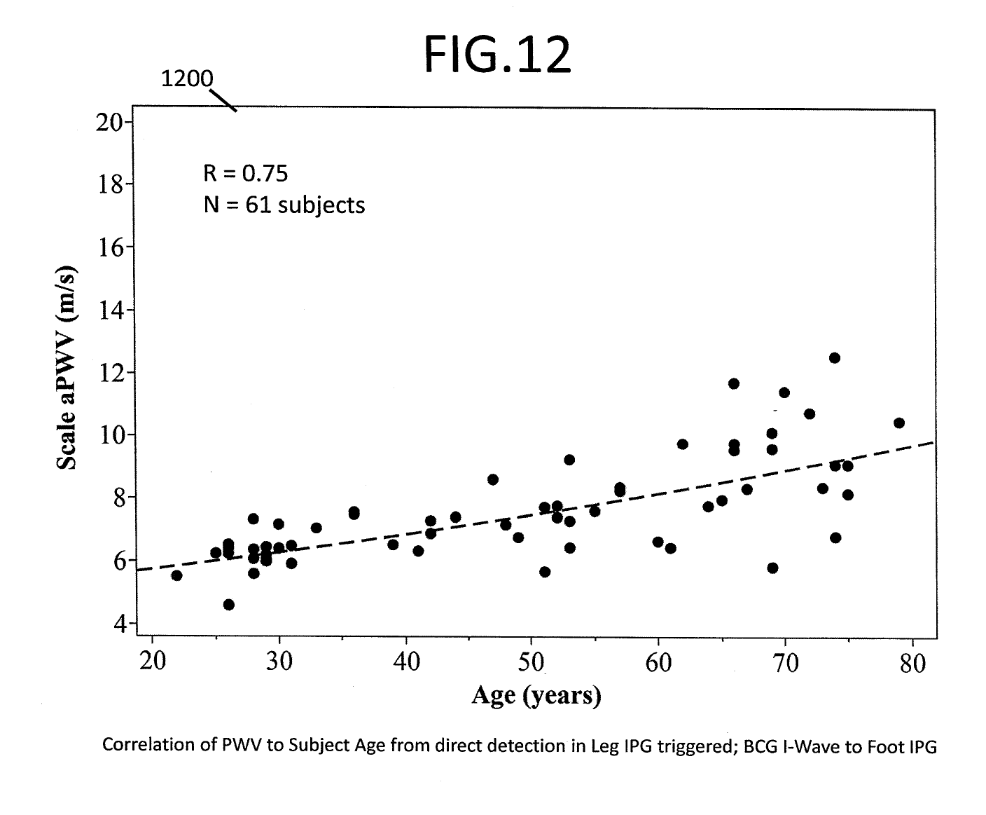

FIG. 11 shows an example graph 1100 of PTT correlations for two detection methods (white dots) Foot IPG only, and (black dots) Dual-IPG method; and FIG. 12 shows an example graph 1200 of PWV obtained from the present disclosure compared to the ages of 61 human test subjects, consistent with various aspects of the present disclosure.

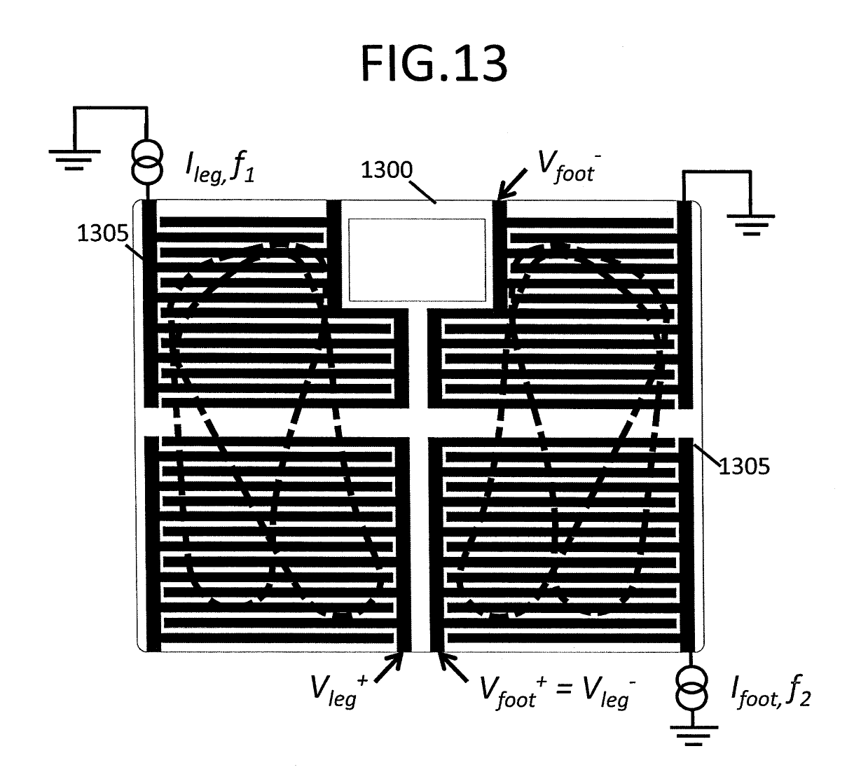

FIG. 13 shows an example of a scale 1300 with integrated foot electrodes 1305 to inject and sense current from one foot to another foot, and within one foot.

FIG. 14a-c shows various examples of a scale 1400 with interleaved foot electrodes 1405 to inject/sense current from one foot to another foot, and measure Foot IPG signals in both feet.

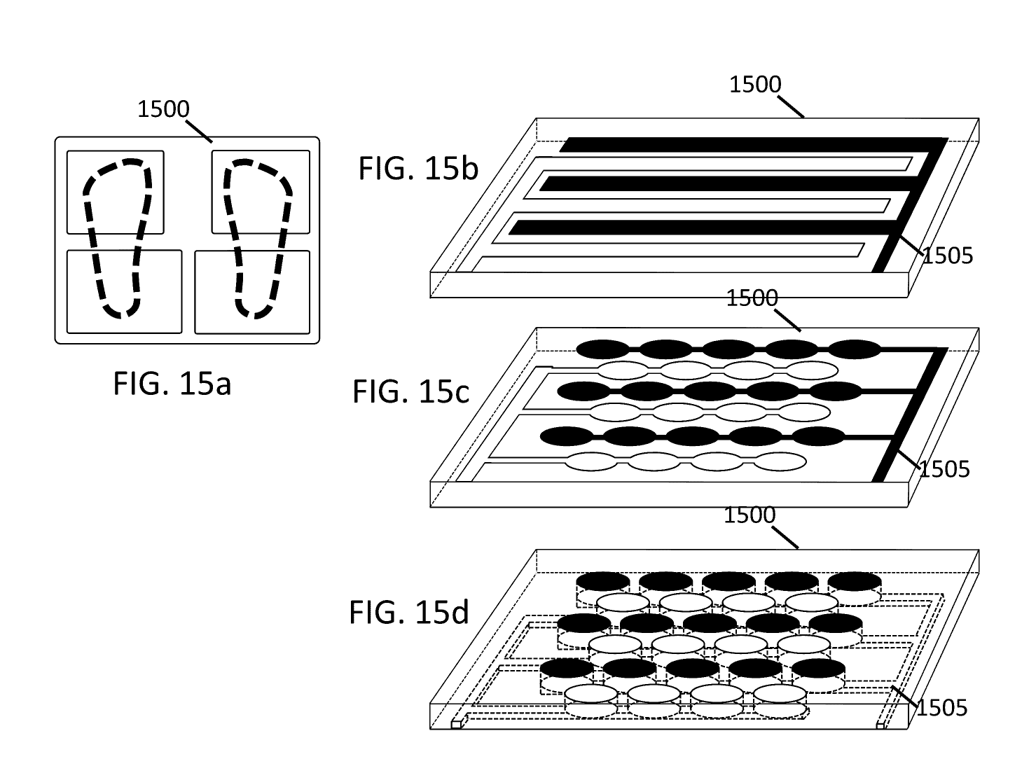

FIGS. 15a-d shows an example breakdown of a scale 1500 with interleaved foot electrodes 1505 to inject and sense current from one foot to another foot, and within one foot.

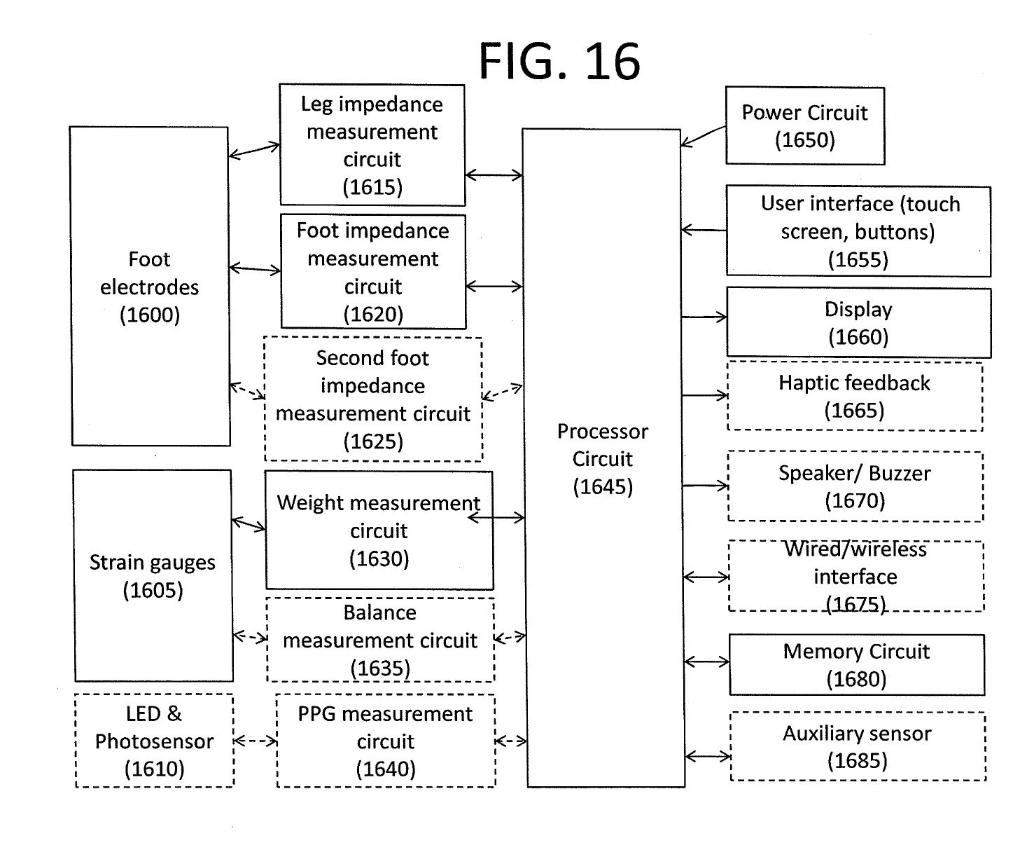

FIG. 16 shows an example block diagram of circuit-based building blocks, consistent with various aspects of the present disclosure. The various circuit-based building blocks shown in FIG. 16 can be implemented in connection with the various aspects discussed herein. In the example shown, the block diagram includes foot electrodes 1600 that can collect the IPG signals. Further, the block diagram includes strain gauges 1605, and an LED/photosensor 1610. The foot electrodes 1600 is configured with a leg impedance measurement circuit 1615, a foot impedance measurement circuit 1620, and an optional second foot impedance measurement circuit 1625. The leg impedance measurement circuit 1615, the foot impedance measurement circuit 1620, and the optional second foot impedance measurement circuit 1625 report the measurements collected to a processor circuitry 1645.

The processor circuitry 1645 collects data from a weight measurement circuit 1630 and an optional balance measurement circuit 1635 that are configured with the strain gauges 1605. Further, an optional photoplethysmogram (PPG) measurement circuit 1640, which collects data from the LED/photosensor 1610, provides data to the processor circuitry 1645.

The processor circuitry 1645 is powered via a power circuit 1650. Further, the processor circuitry 1645 collects user input data from a user interface 1655 (e.g., iPad®, smart phone and/or other remote user handy/CPU with a touch screen and/or buttons). The data collected/measured by the processor circuitry 1645 is shown to the user via a display 1660. Additionally, the data collected/measured by the processor circuitry 1645 can be stored in a memory circuit 1680. Further, the processor circuitry 1645 can optionally control a haptic feedback circuit 1665, a speaker or buzzer 1670, a wired/wireless interface 1675, and an auxiliary sensor 1685.

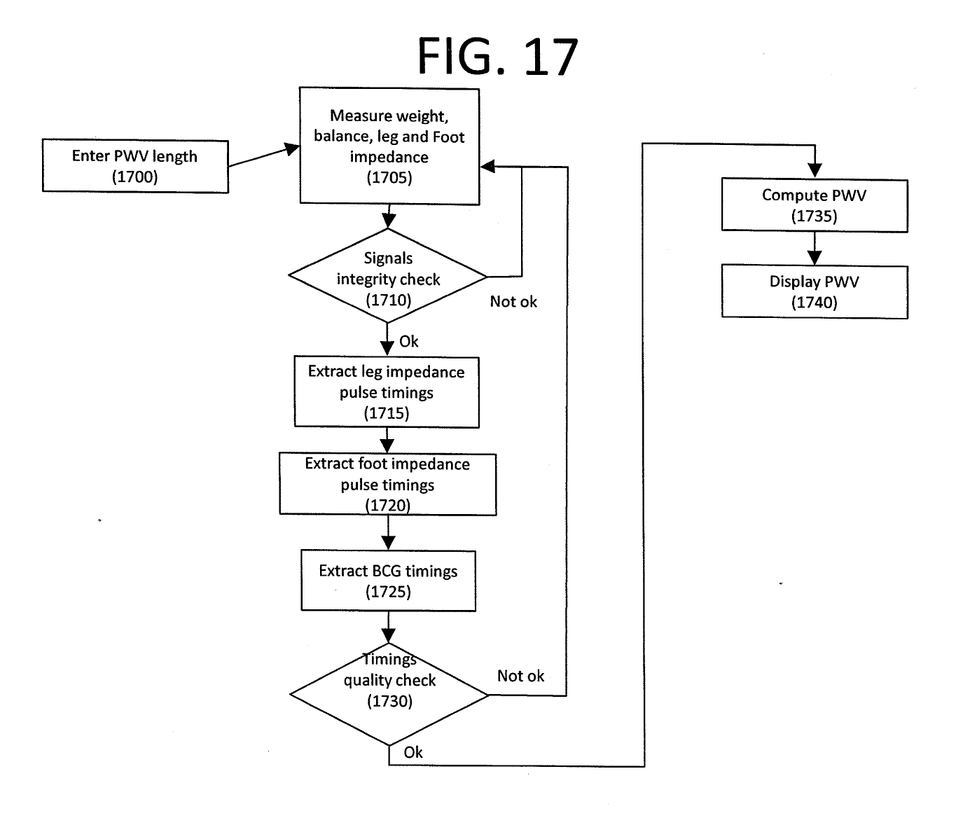

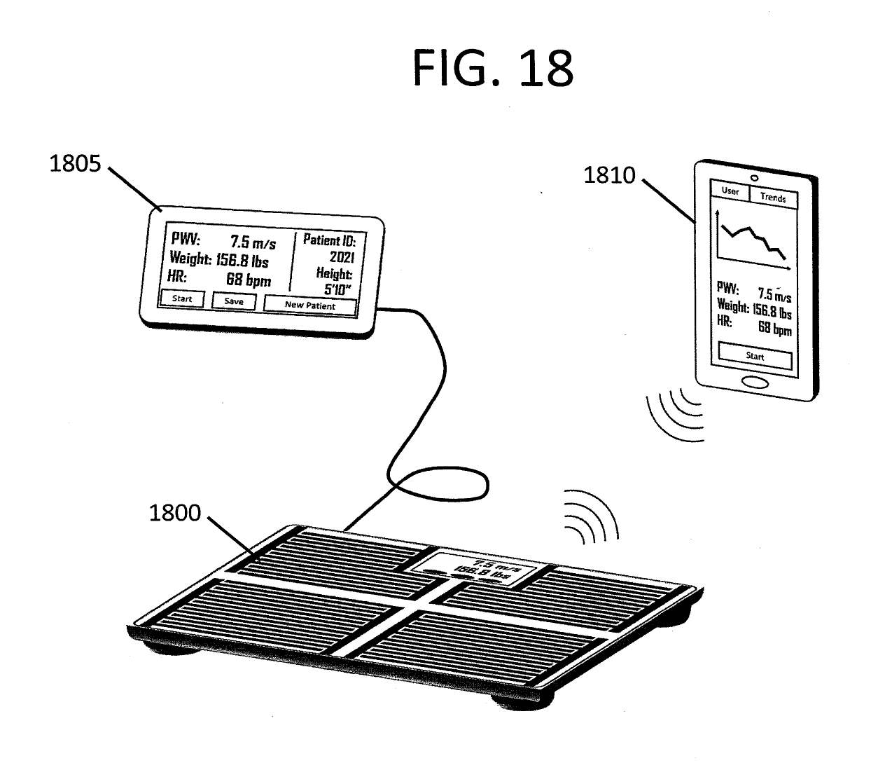

FIG. 17 shows an example flow diagram, consistent with various aspects of the present disclosure. At block 1700, a PWV length is entered. At block 1705, a user's weight, balance, leg, and foot impedance are measured. At 1710, the integrity of signals is checked (e.g., SNR). If the signal integrity check is not met, the user's weight, balance, leg, and foot impedance are measured again (block 1705), if the signals integrity check is met, the leg impedance pulse timings are extracted (as is shown at block 1715). At block 1720, foot impedance and pulse timings are extracted, and at block 1725, BCG timings are extracted. At block 1730, a timings quality check is performed. If the timings quality check is not validated, the user's weight, balance, leg and foot impedance are again measured (block 1705). If the timings quality check is validated, the PWV is calculated (as is shown at block 1735). At block 1740, the PWV is displayed to the user.

FIG. 18 shows an example scale 1800 communicatively coupled to a wireless device, consistent with various aspects of the present disclosure. As described herein, a display 1805 displays the various aspects measured by the scale 1800. The scale, in various embodiments, wirelessly broadcast the measurements to a wireless device 1810. The wireless device 1810, in some aspects, is implemented as an iPad®, smart phone or other CPU to provide input data for configuring and operating the scale.

As an alternative or complementary user interface, the scale includes a foot-controlled user interface which is enabled/implementable by one or more foot-based biometrics (for example, with the user being correlated to previously-entered user weight, and/or foot size/shape). The user foot-based biometric, in some embodiments, is implemented by the user manually entering data (e.g., a password) on the upper surface or display area of the scale. In implementations in which the scale is configured with a haptic, capacitive or flexible pressure-sensing upper surface, the (upper surface/tapping) touching from or by the user is sensed in the region of the surface and processed according to conventional X-Y grid Signal processing in the logic circuitry/CPU that is within the scale. By using one or more of the accelerometers located within the scale at its corners, such user data entry is sensed by each such accelerometer so long as the user's toe, heel or foot pressure associated with each tap provides sufficient force.

In various embodiments, the above discussed user-interface is used with other features described herein for the purpose of storing and securing user sensitive data such as: the configuration data input by the user, the biometric and/or passwords entered by the user, and the user-specific health related data which might include less sensitive data (e.g., the user's weight) and more sensitive data (e.g., the user's scale obtains cardiograms and other data generated by or provided to the scale and associated with the user's symptoms and/or diagnoses). For such user-sensitive data, the above described biometrics are used as directed by the user for indicating and defining protocol to permit such data to be exported from the scale to other remote devices. In more specific embodiments, the scale operates in different modes of data security including, for example: a default mode in which the user's body mass and/or weight is displayed regardless of any biometric which would associate with the specific user standing on the scale; another mode in which complicated data (or data reviewed infrequently) is only exported from the scale under specific manual commands provided to the scale under specific protocols; and another mode or modes in which the user-specific data that is collected from the scale is processed and accessed based on the type of data. Such data categories include categories of different level of importance and/or sensitivities such as the above-discussed high and low level data and other data that might be very specific to a symptom and/or degrees of likelihood for diagnoses. Optionally, the CPU in the scale is also configured to provide encryption of various levels of the user's sensitive data.

For example, in accordance with various embodiments, the above-described foot-controlled user interface is used to provide portions of the user data, clinical indications (e.g., scale-obtained physiological data), generic health information, and/or other feedback to the user. In some embodiments, the scale includes a display configuration filter (e.g., circuitry and/or computer readable medium) configured to discern the data to display to the user and display portion. The display configuration filter discerns which portions of the user data, clinical indications, generic health information and/or other feedback to display to the user on the foot-controlled user interface based on various user demographic information (e.g., age, gender, height, diagnosis) and the amount of data. For example, the generic health information may include an amount of data that if all the data is displayed on the foot-controlled user interface, the data is difficult for a person to read and/or uses multiple display screens.

The display configuration filter discerns portions of the data to display using the scale user interface, such as synopsis of the generic health information (or user data or feedback) and an indication that additional data is displayed on another user device, and other portions to display on the other user device. The other user device is selected by the scale (e.g., the filter) based on various communications settings. The communication settings include settings such as user settings (e.g., the user identifying user devices to output data to), scale-based biometrics (e.g., user configures scale, or default settings, to output data to user devices in response to identifying scale-based biometrics), and/or proximity of the user device (e.g., the scale outputs data to the closest user device among a plurality of user devices and/or in response to the user device being within a threshold distance from the scale), among other settings. For example, the scale determines which portions of the used data, clinical indication, generic health information and/or other feedback to output and outputs the remaining portion of the user data, clinical indication, generic health information and/or other feedback to a particular user device based on user settings/communication authorization (e.g., what user devices are authorized by the user to receive particular user data from the scale), and proximity of the user device to the scale. The determination of which portions to output is based on what type of data is being displayed, how much data is available, and the various user demographic information (e.g., an eighteen year old is able to see better than a fifty year old).

For example, in some specific embodiments, the scale operates in different modes of data security and communication. The different modes of data security and communication are enabled in response to biometrics identified by the user and using the foot-controlled user interface. In some embodiments, the scale is used by multiple users and/or the scale operates in different modes of data security and communication in response to identifying the user and based on biometrics. The different modes of data security and communication include, for example: a first mode (e.g., default mode) in which the user's body mass and/or weight is displayed regardless of any biometric which would associate with the specific user standing on the scale and no data is communicated to external circuitry; a second mode in which complicated/more-sensitive data (or data reviewed infrequently) is only exported from the scale under specific manual commands provided to the scale under specific protocols and in response to a biometric; and third mode or modes in which the user-specific data that is collected from the scale is processed and accessed based on the type of data and in response to a biometric. Such data categories include categories of different levels of importance and/or sensitivities such as the above-discussed high and low level data and other data that might be very specific to a symptom and/or degrees of likelihood for diagnoses. Optionally, the CPU in the scale is also configured to provide encryption of various levels of the user's sensitive data.

In some embodiments, the different modes of data security and communication are enabled in response to recognizing the user standing on the scale using a biometric and operating in a particular mode of data security and communication based on user preferences and/or services activated. For example, the different modes of operation include the default mode (as discussed above) in which certain data (e.g., categories of interest, categories of user-sensitive user data, or historical user data) is not communicated from the scale to external circuitry, a first communication mode in which data is communicated to external circuitry as identified in a user profile, a second or more communication modes in which data is communicated to a different external circuitry for further processing. The different communication modes are enabled based on biometrics identified from the user and user settings in a user profile corresponding with each user.

In a specific embodiment, a first user of the scale may not be identified and/or have a user profile set up. In response to the first user standing on the scale, the scale operates in a default mode. During the default mode, the scale displays the user's body mass and/or weight on the user display and does not output user data. The scale, in various embodiments, displays a prompt (e.g., an icon) on the foot-controlled user interface indicating the first user can establish a user profile. In response to the user selecting the prompt, the scale enters an initialization mode. During the initialization mode, the scale asks the users various questions, such as identification of external circuitry to send data to, identification information of the first user, and/or demographics of the user. The user provides inputs using the foot-controlled user interface to establish various communication modes associated with the user profile and scale-based biometrics to enable the one or more communication modes. The scale further collects user data to identify the scale-based biometrics and stores an indication of the scale-based biometric in the user profile such that during subsequent measurements, the scale recognizes the user and authorizes a particular communication mode. Alternatively, the user provides inputs for the initialization mode using another device that is external to the scale and in communication with the scale (e.g., a cellphone).

A second user of the scale has a user profile set up that indicates the user would like data communicated to a computing device of the user. When the second user stands on the scale, the scale recognizes the second user based on a biometric and operates in a first communication mode. During the first communication mode, the scale outputs at least a portion of the user data to an identified external circuitry. For example, the first communication mode allows the user to upload data from the scale to a user identified external circuitry (e.g., the computing device of the user). The information may include user data and/or user information that has low-user sensitivity, such as user weight and/or bmi. In the first communication mode, the scale performs the processing of the raw sensor data and/or the external circuitry can. For example, the scale sends the raw sensor data and/or additional health information to a user device of the user. The computing device may not provide access to the raw sensor data to the user and/or can send the raw sensor data to another external circuitry for further processing in response to a user input. For example, the computing device can ask the user if the user would like generic health information and/or regulated health information as a service. In response to receiving an indication the user would like the generic health information and/or regulated health information, the computing device outputs the raw sensor data and/or non-regulated health information to another external circuitry for processing, providing to a physician for review, and controlling access, as discussed above.

In one or more additional communication modes, the scale outputs raw sensor data to an external circuitry for further processing. For example, during a second communication mode and a third communication, the scale sends the raw sensor data and/or other data to external circuitry for processing. Using the above-provided example, a third user of the scale has a user profile set up that indicates the third user would like scale-obtained data to be communicated to an external circuitry for further processing, such as to determine generic health information. When the third user stands on the scale, the scale recognizes the third user based on one or more biometrics and operates in a second communication mode. During the second communication mode, the scale outputs raw sensor data to the external circuitry. The external circuitry identifies one or more risks, and, optionally, derives generic health information. In some embodiments, the external circuitry outputs the generic health information to the scale. The scale, in some embodiments, displays a synopsis of the generic health information and/or outputs a full version of the generic health information to another user device for display (such as, using the filter described above) and/or an indication that generic health information can be accessed.

A fourth user of the scale has a user profile set up that indicates the fourth user has enabled a service to access regulated health information. When the fourth user stands on the scale, the scale recognizes the user based on one or more biometrics and operates in a fourth communication mode. In the fourth communication mode, the scale outputs raw sensor data to the external circuitry, and the external circuitry processes the raw sensor data and controls access to the data. For example, the external circuitry may not allow access to the regulated health information until a physician reviews the information. In some embodiments, the external circuitry outputs data to the scale, in response to physician review. For example, the output data can include the regulated health information and/or an indication that regulated health information is ready for review. The external circuitry may be accessed by the user, using the scale and/or another user device. In some embodiments, using the foot-controlled user-interface of the scale, the scale displays the regulated health information to the user. The scale, in some embodiments, displays a synopsis of the regulated health information (e.g., clinical indication) and outputs the full version of regulated health information to another user device for display (such as, using the filter described above) and/or an indication that the regulated health information can be accessed to the scale to display. In various embodiments, if the scale is unable to identify a particular (high security) biometric that enables the fourth communication mode, the scale may operate in a different communication mode and may still recognize the user. For example, the scale may operate in a default communication mode in which the user data collected by the scale is stored in a user profile corresponding to the fourth user and on the scale. In some related embodiments, the user data is output to the external circuitry at a different time.

Although the present embodiments illustrates a number of security and communication modes, embodiments in accordance with the present disclosure can include additional or fewer modes. Furthermore, embodiments are not limited to different modes based on different users. For example, a single user may enable different communication modes in response to particular biometrics of the user identified and/or based on user settings in a user profile.

In various embodiments, the scale defines a user data table that defines types of user data and sensitivity values of each type of user data. In specific embodiments, the foot-controlled user interface displays the user data table. In other specific embodiments a user interface of a smartphone, tablet, and/or other computing device displays the user data table. For example, a wired or wireless tablet is used, in some embodiments, to display the user data table. The sensitivity values of each type of user data, in some embodiments, define in which communication mode(s) the data type is communicated and/or which biometric is used to enable communication of the data type. In some embodiments, a default or pre-set user data table is displayed and the user revises the user data table using the foot-controlled user interface. The revisions are in response to user inputs using the user's foot and/or contacting or moving relative to the foot-controlled user interface. Although the embodiments are not so limited, the above (and below) described control and display is provided using a wireless or wired tablet or other computing device as a user interface. The output to the wireless or wired tablet, as well as additional external circuitry, is enabled using biometrics. For example, the user is encouraged, in particular embodiments, to configure the scale with various biometrics. The biometric include scale-based biometrics and biometrics from the tablet or other user computing device. The biometric, in some embodiments, used to enable output of data to the tablet and/or other external circuitry includes a higher integrity biometric (e.g., higher likelihood of identifying the user accurately) than a biometric used to identify the user and stored data on the scale.

An example user data table is illustrated below:

| | | | | | Scale-stored |
|---|---|---|---|---|---|
| | | Body | | | suggestions |
| User-data | | Mass | User-Specific | Physician-Provided | (symptoms & |
| Type | Weight | Index | Advertisements | Diagnosis/Reports | diagnosis) |
| Sensitivity | 1 | 3 | 5 | 10 | 9 |
| (10 = highest, | | | | | |
| 1 = lowest) | | | | | |

The above-displayed table is for illustrative purposes and embodiments in accordance with the present disclosure can include additional user-data types than illustrated, such as cardiogram characteristics, clinical indications, physiological parameters, user goals, demographic information, etc. In various embodiments, the user data table includes additional rows than illustrated. The rows, in specific embodiments, include different data input sources and/or sub-data types (as discussed below). Data input sources include source of the data, such as physician provided, input from the Internet, user provided, from the external circuitry. The different data from the data input sources, in some embodiments, is used alone or in combination.

In accordance with various embodiments, the scale uses a cardiogram of the user and/or other scale-obtained biometrics to differentiate between two or more users. The scale-obtained data includes health data that is user-sensitive, such that unintentional disclosure of scale-obtained data is not desired. Differentiating between the two or more users and automatically communicating (e.g., without further user input) user data responsive to scale-obtained biometrics, in various embodiments, provides a user-friendly and simple way to communicate data from a scale while avoiding and/or mitigating unintentional (and/or without user consent) communication. For example, the scale, such as during an initialization mode for each of the two or more users and as previously discussed, collects user data to identify the scale-based biometrics and stores an indication of the scale-based biometrics in a user profile corresponding with the respective user. During subsequent measurements, the scale recognizes the particular user by comparing collected signals to the indication of the scale-based biometrics in the user profile. The scale, for example, compares the collected signals to each user profile of the two or more users and identifies a match between the collected signals and the indication of the scale-based biometrics. A match, in various embodiments, is within a range of values of the indication stored. Further, in response to verifying the scale-based biometric(s), a particular communication mode is authorized.

In accordance with a number of embodiments, the scale identifies one or more of the multiple users of the scale that have priority user data. The user data with a priority, as used herein, includes an importance of the user and/or the user data. In various embodiments, the importance of the user is based on parameter values identified and/or user goals, such as the user is an athlete and/or is using the scale to assist in training for an event (e.g., marathon) or is using the scale for other user goals (e.g., a weight loss program). Further, the importance of the user data is based on parameters values and/or user input data indicating a diagnosis of a condition or disease and/or a risk of the user having the condition or disease based on the scale-obtained data. For example, the scale-obtained data of a first user indicates that the user is overweight, recently had an increase in weight, and has a risk of having atrial fibrillation. The first user is identified as a user corresponding with priority user data. A second user of the scale has scale-obtained data indicating a decrease in recovery parameters (e.g., time to return to baseline parameters) and the user inputs an indication that they are training for a marathon. The second user is also identified as a user corresponding with priority user data. The scale displays indications to user with the priority user data, in some embodiments, on how to use to the scale to communicate the user data to external circuitry for further processing, correlation, and/or other features, such as social network connections. Further, the scale, in response to the priority, displays various feedback to the user, such as user-targeted advertisements and/or suggestions. In some embodiments, only users with priority user data have data output to the external circuitry to determine risks, although embodiments in accordance with the present disclosure are not so limited.

In some embodiments, one or more users of the scale have multiple different scale-obtained biometrics used to authorize different communication modes. The different scale-obtained biometrics are used to authorize communication of different levels of user sensitive data, such as the different user-data types and sensitivity values as illustrated in the above-table. For example, in some specific embodiments, the different scale-obtained biometrics include a high security biometric, a medium security biometric, and a low security biometric. Using the above illustrated table as an example, the three different biometrics are used to authorize communication of the user-data types of the different sensitivity values. For instance, the high security biometric authorizes communication of user-data types with sensitivity values of 8-10, the medium security biometric authorizes communication of user-data types with sensitivity values of 4-7, and the low security biometric authorizes communication of user-data types with sensitivity values of 1-3. The user, in some embodiments, can adjust the setting of the various biometrics and authorization of user-data types.

In a specific example, low security biometrics includes estimated weight (e.g., a weight range), and a toe tap on the foot-controlled user interface. Example medium security biometrics includes one or more the low security biometric in addition to length and/or width of the user's foot, and/or a time of day or location of the scale. For example, as illustrated by FIGS. 2 and 13 and discussed with regard to FIG. 3c, the scale includes impedance electrodes that are interleaved and engage the feet of the user. The interleaved electrodes assist in providing measurement results that are indicative of the foot length, foot width, and type of arch. Further, a specific user, in some embodiments, may use the scale at a particular time of the day and/or authorize communication of data at the particular time of the day, which is used to verify identity of the user and authorize the communication. The location of scale, in some embodiments, is based on Global Positioning System (GPS) coordinates and/or a Wi-Fi code. For example, if the scale is moved to a new house, the Wi-Fi code used to communicate data externally from the scale changes. Example high security biometrics include one or more low security biometrics and/or medium security biometrics in addition to cardiogram characteristics and, optionally, a time of day and/or heart rate. Example cardiogram characteristics include a QRS complex, and QRS complex and P/T wave.

In various embodiments, the user adjusts the table displayed above to revise the sensitivity values of each data type. Further, although the above-illustrated table includes a single sensitivity value for each data type, in various embodiments, one or more of the data types are separated into sub-data types and each sub-data type has a sensitivity value. As an example, the user-specific advertisement is separated into: prescription advertisement, external device advertisements, exercise advertisements, and diet plan advertisement. Alternatively and/or in addition, the sub-data types for user-specific advertisement include generic advertisements based on a demographic of the user and advertisements in response to scale collected data (e.g., advertisement for a device in response to physiologic parameters), as discussed further herein.

For example, weight data includes the user's weight and historical weight as collected by the scale. In some embodiments, weight data includes historical trends of the user's weight and correlates to dietary information and/or exercise information, among other user data. Body mass index data, includes the user's body mass index as determined using the user's weight collected by the scale and height. In some embodiments, similar to weight, body mass index data includes history trends of the user's body mass index and correlates to various other user data.

User-specific advertisement data includes various prescriptions, exercise plans, dietary plans, and/or other user devices and/or sensors for purchase, among other advertisements. The user-specific advertisements, in various embodiments, are correlated to input user data and/or scale-obtained data. For example, the advertisements include generic advertisements that are relevant to the user based on a demographic of the user. Further, the advertisements include advertisements that are responsive to scale collected data (e.g., physiological parameter includes a symptom or problem and advertisement is correlated to the symptom or problem). A number of specific examples include advertisements for beta blockers to slow heart rate, advertisements for a user wearable device (e.g., Fitbit®) to monitor heart rate, and advertisements for a marathon exercise program (such as in response to an indication the user is training for a marathon), etc.

Physician provided diagnosis/report data includes data provided by a physician and, in various embodiments, is in responsive to the physician reviewing the scale-obtained data. For example, the physician provided diagnosis/report data includes diagnosis of a disorder/condition by a physician, prescription medication prescribed by a physician, and/or reports of progress by a physician, among other data. In various embodiments, the physician provided diagnosis/reports are provided to the scale from external circuitry, which includes and/or accesses a medical profile of the user.

Suggestion data includes data that provides suggestions or advice for symptoms, diagnosis, and/or user goals. For example, the suggestions include advice for training that is user specific (e.g., exercise program based on user age, weight, and cardiogram data or exercise program for training for an event or reducing time to complete an event, such as a marathon), suggestions for reducing symptoms including dietary, exercise, and sleep advice, and/or suggestions to see a physician, among other suggestions. Further, the suggestions or advice include reminders regarding prescriptions. For example, based on physician provided diagnosis/report data and/or user inputs, the scale identifies the user is taking a prescription medication. The identification includes the amount and timing of when the user takes the medication, in some embodiments. The scale reminds the user and/or asks for verification of consumption of the prescription medication using the foot-controlled user interface.

As further specific examples, recent discoveries may align and associate different attributes of scale-based user data collected by the scale to different tools, advertisements, and physician provided diagnosis. For example, it has recently been discovered that atrial fibrillation is more directly correlated with obesity. The scale collects various user data and monitors weight and various components/symptoms of atrial fibrillation. In a specific embodiment, the scale recommends/suggests to the user to: closely monitor weight, recommends a diet, goals for losing weight, and correlates weight gain and losses for movement in cardiogram data relative to arrhythmia. The movement in cardiogram data relative to arrhythmia, in specific embodiments, is related to atrial fibrillation. For example, atrial fibrillation is associated with indiscernible p-waves and beat to beat fluctuations. Thereby, the scale correlates weight gain/loss with changes in amplitude (e.g., discernibility) of a p-wave of a cardiogram (preceding a QRS complex) and changes in beat to beat fluctuations.

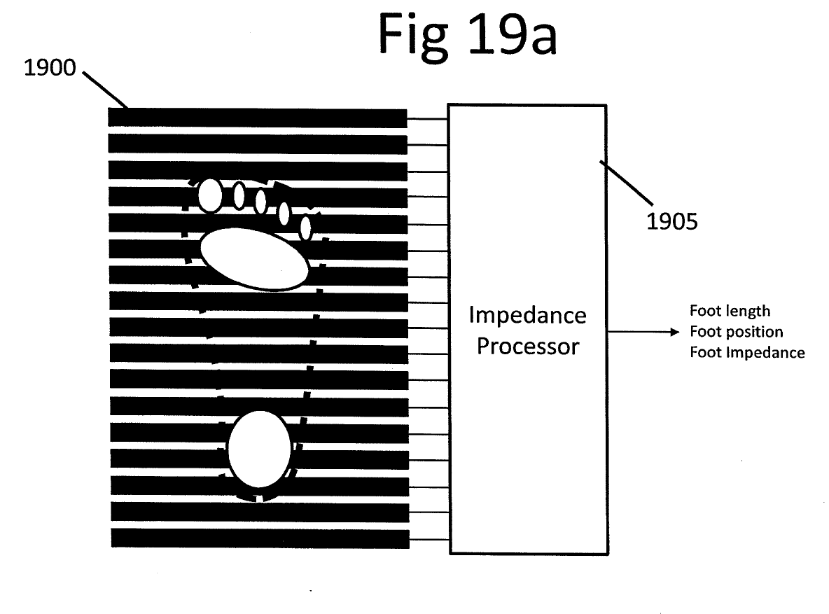

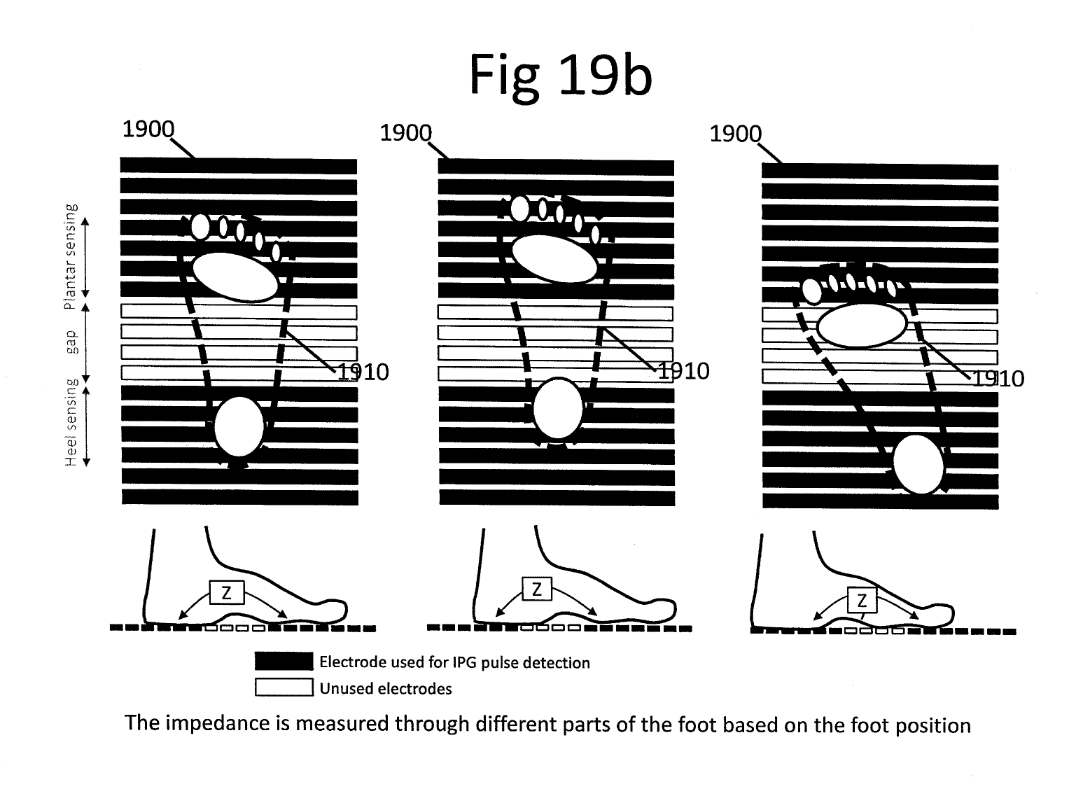

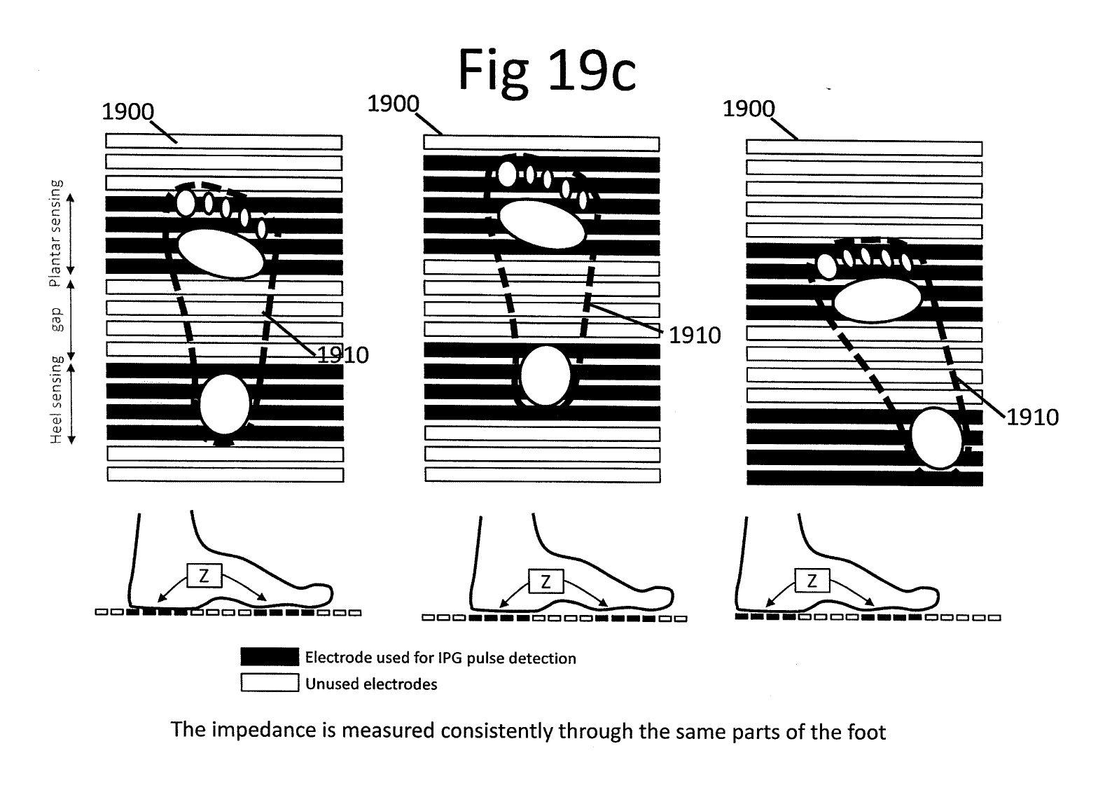

FIGS. 19a-c show example impedance as measured through different parts of the foot based on the foot position, consistent with various aspects of the present disclosure. For instance, example impedance measurement configurations may be implemented using a dynamic electrode configuration for measurement of foot impedance and related timings. Dynamic electrode configuration may be implemented using independently-configurable electrodes to optimize the impedance measurement. As shown in FIG. 19a, interleaved electrodes 1900 are connected to an impedance processor circuit 1905 to determine foot length, foot position, and/or foot impedance. As is shown in FIG. 19b, an impedance measurement is determined regardless of foot position 1910 based on measurement of the placement of the foot across the electrodes 1900. This is based in part in the electrodes 1900 that are engaged (blackened) and in contact with the foot (based on the foot position 1910), which is shown in FIG. 19c.

More specifically regarding FIG. 19a, configuration includes connection/de-connection of the individual electrodes 1900 to the impedance processor circuit 1905, their configuration as current-carrying electrodes (injection or return), sense electrodes (positive or negative), or both. The configuration is preset based on user information, or updated at each measurement (dynamic reconfiguration) to optimize a given parameter (impedance SNR, measurement location). The system algorithmically determines which electrodes under the foot to use in order to obtain the highest SNR in the pulse impedance signal. Such optimization algorithm may include iteratively switching configurations and measuring the impedance, and selecting the best suited configuration. Alternatively, the system first, through a sequential impedance measurement between each individual electrode 1900 and another electrode in contact with the body (such as an electrode in electrode pair 205 on the other foot), determine which electrodes are in contact with the foot. By determining the two most apart electrodes, the foot size is determined. Heel location can be determined in this manner, as can other characteristics such as foot arch type. These parameters are used to determine programmatically (in an automated manner by CPU/logic circuitry) which electrodes are selected for current injection and return (and sensing if a Kelvin connection issued) to obtain the best foot IPG.

In various embodiments involving the dynamically reconfigurable electrode array 1900/1905, an electrode array set is selected to measure the same portion/segment of the foot, irrespective of the foot location on the array. FIG. 19b illustrates the case of several foot positions on a static array (a fixed set of electrodes are used for measurement at the heel and plantar/toe areas, with a fixed gap of an inactive electrode or insulating material between them). Depending on the position of the foot, the active electrodes are contacting the foot at different locations, thereby sensing a different volume/segment of the foot. If the IPG is used by itself (e.g., for heart measurement), such discrepancies may be non-consequential. However, if timings derived from the IPG are referred to other timings (e.g., R-wave from the ECG, or specific timing in the BCG), such as for the calculation of a PTT or PWV, the small shifts in IPG timings due to the sensing of slightly different volumes in the foot (e.g., if the foot is not always placed at the same position on the electrodes) can introduce an error in the calculation of the interval. With respect to FIG. 19b, the timing of the peak of the IPG from the foot placement on the right (sensing the toe/plantar region) is later than from the foot placement on the left, which senses more of the heel volume (the pulse reaches first the heel, then the plantar region). Factors influencing the magnitude of these discrepancies include foot shape (flat or not) and foot length.

Various embodiments address challenges relating to foot placement. FIG. 19c shows an example embodiment involving dynamic reconfiguration of the electrodes to reduce such foot placement-induced variations. As an example, by sensing the location of the heel first (as described above), it is possible to activate a subset of electrodes under the heel, and another subset of electrodes separated by a fixed distance (1900). The other electrodes (e.g., unused electrodes) are left disconnected. The sensed volume will therefore be the same, producing consistent timings. The electrode configuration leading to the most consistent results may be informed by the foot impedance, foot length, the type of arch (all of which can be measured by the electrode array as shown above), but also by the user ID (foot information can be stored for each user, then looked up based on automatic user recognition or manual selection (e.g., in a look-up-table stored for each user in a memory circuit accessible by the CPU circuit in the scale).

In certain embodiments, the apparatus measures impedance using a plurality of electrodes contacting one foot and with at least one other electrode (typically many) at a location distal from the foot. The plurality of electrodes (contacting the one foot) is arranged on the platform and in a pattern configured to inject current signals and sense signals in response thereto, for the same segment of the foot so that the timing of the pulse-based measurements does not vary because the user placed the one foot at a slightly different position on the platform or scale. In FIG. 19a, the foot-to-electrode locations for the heel are different locations than that shown in FIGS. 19b and 19c. As this different foot placement can occur from day to day for the user, the timing and related impedance measurements are for the same (internal) segment of the foot. By having the processor circuit inject current and sense responsive signals to first locate the foot on the electrodes (e.g., sensing where positions of the foot's heel plantar regions and/or toes), the pattern of foot-to-electrode locations permits the foot to move laterally, horizontally and both laterally and horizontally via the different electrode locations, while collecting impedance measurements relative to the same segment of the foot.

The BCG/IPG system can be used to determine the PTT of the user, by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. In certain embodiments, the BCG/IPG system is used to determine the PWV of the user, by identification of the characteristic length representing the length of the user's arteries, and by identification of the average I-Wave or derivative timing near the I-Wave from a plurality of BCG heartbeat signals obtained simultaneously with the Dual-IPG measurements of the present disclosure to determine the relative PTT along an arterial segment between the ascending aortic arch and distal pulse timing of the user's lower extremity. The system of the present disclosure and alternate embodiments may be suitable for determining the arterial stiffness (or arterial compliance) and/or cardiovascular risk of the user regardless of the position of the user's feet within the bounds of the interleaved electrodes. In certain embodiments, the weighing scale system incorporated the use of strain gage load cells and six or eight electrodes to measure a plurality of signals including: bodyweight, BCG, body mass index, fat percentage, muscle mass percentage, and body water percentage, heart rate, heart rate variability, PTT, and PWV measured simultaneously or synchronously when the user stands on the scale to provide a comprehensive analysis of the health and wellness of the user.

In other certain embodiments, the PTT and PWV are computed using timings from the Leg IPG or Foot IPG for arrival times, and using timings from a sensor located on the upper body (as opposed to the scale measuring the BCG) to detect the start of the pulse. Such sensor may include an impedance sensor for impedance cardiography, a hand-to-hand impedance sensor, a photoplethysmogram on the chest, neck, head, arms or hands, or an accelerometer on the chest (seismocardiograph) or head.

Communication of the biometric information is another aspect of the present disclosure. The biometric results from the user are stored in the memory on the scale and displayed to the user via a display on the scale, audible communication from the scale, and/or the data is communicated to a peripheral device such as a computer, smart phone, tablet computing device. The communication occurs to the peripheral device with a wired connection, or can be sent to the peripheral device through wireless communication protocols such as Bluetooth or WiFi. Computations such as signal analyses described therein may be carried out locally on the scale, in a smartphone or computer, or in a remote processor (cloud computing).

Other aspects of the present disclosure are directed toward apparatuses or methods that include the use of at least two electrodes that contacts feet of a user. Further, circuitry is provided to determine a pulse arrival time at the foot based on the recording of two or more impedance signals from the set of electrodes. Additionally, a second set of circuitry is provided to extract a first pulse arrival time from a first impedance signal and use the first pulse arrival time as a timing reference to extract and process a second pulse arrival time in a second impedance signal.

Reference may also be made to published patent documents U.S. Patent Publication 2010/0094147 and U.S. Patent Publication 2013/0310700, which are, together with the references cited therein, herein fully incorporated by reference for the purposes of sensors and sensing technology. The aspects discussed therein may be implemented in connection with one or more of embodiments and implementations of the present disclosure (as well as with those shown in the figures). In view of the description herein, those skilled in the art will recognize that many changes may be made thereto without departing from the spirit and scope of the present disclosure.

Terms to exemplify orientation, such as upper/lower, left/right, top/bottom and above/below, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

As illustrated herein, various circuit-based building blocks and/or modules may be implemented to carry out one or more of the operations/activities described herein shown in the block-diagram-type figures. In such contexts, these building blocks and/or modules represent circuits that carry out these or related operations/activities. For example, in certain embodiments discussed above (such as the pulse circuitry modularized as shown in FIGS. 3a-b), one or more blocks/modules are discrete logic circuits or programmable logic circuits for implementing these operations/activities, as in the circuit blocks/modules shown. In certain embodiments, the programmable circuit is one or more computer circuits programmed to execute a set (or sets) of instructions (and/or configuration data). The instructions (and/or configuration data) can be in the form of firmware or software stored in and accessible from a memory circuit. As an example, first and second modules/blocks include a combination of a CPU hardware-based circuit and a set of instructions in the form of firmware, where the first module/block includes a first CPU hardware circuit with one set of instructions and the second module/block includes a second CPU hardware circuit with another set of instructions.

Various embodiments are implemented in accordance with the underlying Provisional application (Ser. No. 62/266,440), entitled “Scale-based User-Physiological Social Grouping System”, filed Dec. 11, 2015, Provisional application (Ser. No. 62/258,238), entitled “Condition or Treatment Assessment Methods and Platform Apparatuses”, filed Nov. 20, 2015, and Provisional application (Ser. No. 62/266,523) entitled “Social Grouping Using a User-Specific Scale-Based Enterprise System”, filed Dec. 11, 2015, to which benefit is claimed and which are fully incorporated herein by reference. For instance, embodiments herein and/or in the provisional applications may be combined in varying degrees (including wholly). Reference may also be made to the experimental teachings and underlying references provided in the underlying provisional application. Embodiments discussed in the provisional applicants are not intended, in any way, to be limiting to the overall technical disclosure.

Based upon the above discussion and illustrations, those skilled in the art will readily recognize that various modifications and changes may be made to the present disclosure without strictly following the exemplary embodiments and applications illustrated and described herein. For example, the input terminals as shown and discussed may be replaced with terminals of different arrangements, and different types and numbers of input configurations (e.g., involving different types of input circuits and related connectivity). Further, the various features and operations/actions, in accordance with various embodiments, can be combined with various different features and operations/actions and in various combinations. For example, the feature of grouping users into social groups and providing access to the social groups can be used in combination with discerning which data to display on the user interface of the scale and which data to display on another device. Such modifications do not depart from the true spirit and scope of the present disclosure, including that set forth in the following claims.

What is claimed is:

1. An apparatus comprising:
a weighing scale including:
a platform including force sensor circuitry and a plurality of electrodes integrated with the platform, and configured and arranged for engaging a user with electrical signals and collecting signals indicative of the user's identity and cardio-related physiologic data while the user is standing on the platform; and
processing circuitry, including a CPU and a memory circuit with user-corresponding data stored in the memory circuit, configured and arranged under the platform, the processing circuitry being electrically integrated with the force sensor circuitry and the plurality of electrodes and being configured to collect cardio-related physiologic data from the user while the user is standing on the platform and output at least portions of the cardio-related physiologic data as user data; and
external circuitry configured and arranged to receive user data from a plurality of weighing scales include the weighing scale and to:
pool user data for a plurality of users of a plurality of scales into user data sets for each user;
identify a subset of users of the plurality of users with correlations between user data sets based on the pooled used data;
identify and normalize user data from the user data sets of the subsets of users based on prioritization data and normalization data; and
provide the subsets of users with access to a social group via respective scales of the subset of users, wherein providing access to the social group includes selective access to the normalized user data from the user data sets.

2. The apparatus of claim 1, wherein the external circuitry provides the access to the social group by provide the subset of users access to a report or social media page that includes the subset of users without user identification information.

3. The apparatus of claim 1, wherein the external circuitry provides the access by providing a forum, blog, and/or webpage that has reports and/or dashboards with at least portions of the user data sets that are populated therein and correspond with the subset of users.

4. The apparatus of claim 1, wherein the external circuitry identifies the correlations based on similar risks that user for a condition using the user data.

5. The apparatus of claim 1, wherein access to the social group includes providing access to forum, blog, and/or webpage display various reports and/or dashboards indicating successes and failures, treatments, and/or progress of the user of the social group based on scale-obtained data over a period of time.

6. The apparatus of claim 1, wherein the processing circuitry configured and arranged to identify the user by verifying a scale-based biometric of the user using the signals indicative of the user's identity and a user profile corresponding to the user.

7. The apparatus of claim 1, the weighing scale further includes:
a user display configured and arranged with the platform and the plurality of electrodes to output data to the user while the user is standing on the platform; and
an output circuit configured and arranged to receive user data, including the cardio-related physiologic data obtained by the scale and cardio-related physiologic data from another user device, in response, aggregate and output at least a portion of the user data and the cardio-related physiologic data from the weighing scale to external circuitry.

8. The apparatus of claim 1, wherein the processing circuitry is configured and arranged to collect user data for more than one user, prioritize the more than one users based on the user data and user goals, and outputs the user data to identify social groups, and for other services, based on the priority of the users.

9. The apparatus of claim 8, wherein the scale collects signals from the plurality of users over time and associates the collected signals with each respective user among the plurality of users by verifying scale-based biometrics of the users using the signals indicative of the user's identity and each respective user profile, wherein the processing circuitry is further configured and arranged to:
identify users among the plurality of users that have cardio-related physiologic data with a threshold priority as compared to the remaining plurality of users including:
identifying a first user among the plurality of users that has cardio-related physiologic data indicative of an athlete; and
identifying a second user among the plurality of users that has cardio-related physiologic data indicative of a medical issue; and
output the user data corresponding to the first user and the second user to the external circuitry.

10. An apparatus comprising:
a weighing scale including:
a platform including force sensor circuitry and a plurality of electrodes integrated with the platform, and configured and arranged for engaging a user with electrical signals and collecting signals indicative of the user's identity and cardio-related physiologic data while the user is standing on the platform;
a user display configured and arranged to display data to a user while the user is standing on the weighing scale, and
processing circuitry, including a CPU and a memory circuit with user-corresponding data stored in the memory circuit, configured and arranged within the scale and under the platform, the processing circuit being electrically integrated with the force sensor circuitry and the plurality of electrodes and being configured to process data obtained by the force sensor circuitry while the user is standing on the platform and therefrom generate cardio-related physiologic data corresponding to the collected signals, the processing circuitry configured and arranged to identify the user by verifying a scale-based biometric of the user using the signals indicative of the user's identity and a user profile corresponding to the user;
external circuitry, including processing circuitry and a memory circuitry, configured and arranged to receive user data from a plurality of scales, the plurality of weighing scales including the weighing scale, and to:
pool user data for a plurality of users of a plurality of scales into user data sets for each user;
identify a subset of users of the plurality of users with correlations between user data sets based on the pooled used data, wherein at least one correlations includes user that are experiencing symptoms, conditions, or treatments based on the user data;
identify and normalize user data from the user data sets of the subsets of users based on prioritization data and normalization data; and
provide the subsets of users with access to a social group via respective scales of the subset of users, wherein providing access to the social group includes selective access to the normalized user data from the user data sets.

11. The apparatus of claim 10, wherein the weighing scale is configured and arranged to receive data from a plurality of user devices and correlated the respective data with user profiles corresponding to the plurality of users in response to identification of the users using scale-based biometrics and data within the received data from the plurality of user devices, wherein the processing circuitry is configured and arranged to aggregate data corresponding to a particular user and output the aggregated data to the external circuitry in response to verifying a scale-based biometric from the user that authorizes a communication between the scale and the external circuitry.

12. The apparatus of claim 10, wherein the processing circuitry is configured and arranged to collects signal from a plurality of users over time and associate the respective collected signals with each respective user among the plurality of users by verifying scale-based biometrics of the users using signals indicative of the user's identity and each respective user profile.

13. The apparatus of claim 12, wherein the processing circuitry is further configured and arranged to:
identify users among the plurality of users that have cardio-related physiologic data with a threshold priority as compared to the remaining plurality of users including:
identifying a first user among the plurality of users that has cardio-related physiologic data indicative of an athlete; and
identifying a second user among the plurality of users that has cardio-related physiologic data indicative of a medical issue; and
output the user data corresponding to the first user and the second user to the external circuitry.

14. The apparatus of claim 10, wherein the scale-based biometric used to identity the user includes a cardiogram and wherein the processing circuitry is configured and arranged to add an identifier to the user data that is indicative of an identity of the weighing scale and the user.

15. A method and/or apparatus as is consistent with claim 1 and/or one or more of the embodiments disclosed herein.
